(19) United States
(12) Patent Application Publication
(10) Pub. No.: US 2016/0075718 A1
(43) Pub. Date: Mar. 17, 2016

(54) Organic Compound, Light-Emitting Element, Light-Emitting Device, Electronic Device, and Lighting Device

(71) Applicant: Semiconductor Energy Laboratory Co., Ltd., Kanagawa-ken (JP)

(72) Inventors: Satomi Mitsumori, Atsugi (JP); Yuko OHE, lsehara (JP); Takao HAMADA, Atsugi (JP)

(73) Assignee: Semiconductor Energy Laboratory Co., Ltd., Kanagawa-ken (JP)

(21) Appl. No.: 14/844,284

(22) Filed: Sep. 3, 2015

(30) Foreign Application Priority Data
Sep. 5, 2014 (JP) 2014-181112

(51) Int. Cl. C07D 495/04; H01L 27/32; C09K 11/06; H01L 51/00; C07D 491/048

(52) U.S. Cl. CPC C07D 495/04; H01L 51/0067; H01L 51/0071; C07D 491/048; C09K 11/06; H01L 27/3244; C09K 2211/1092; C09K 2211/1029; C09K 2211/1011; C09K 2211/1007; C09K 2211/1088; H01L 51/5012

(57) ABSTRACT

An organic compound represented by a general formula (G1).

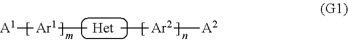

In the general formula (G1), Het represents a substituted or unsubstituted π-electron deficient divalent heteroaromatic group having 4 to 36 carbon atoms, Ar¹ and Ar² each independently represent a substituted or unsubstituted arylene group having 6 to 25 carbon atoms, in and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

### BACKGROUND OF THE INVENTION

1. Field of the Invention

The present invention relates to an object, a method, or a fabrication method. In addition, the present invention relates to a process, a machine, manufacture, or a composition of matter. In particular, one embodiment of the present invention relates to a semiconductor device, a display device, a light-emitting device, a driving method thereof, or a fabrication method thereof. In particular, one embodiment of the present invention relates to an organic compound and a novel method of synthesizing the same. In addition, one embodiment of the present invention relates to a light-emitting element, a light-emitting device, an electronic device, and a lighting device that include the organic compound.

2. Description of the Related Art

A light-emitting element using an organic compound as a luminous body, which has features such as thinness, lightness, high-speed response, and DC drive at low voltage, is expected to be used in a next-generation flat panel display. In particular, a display device in which light-emitting elements are arranged in matrix is considered to have advantages in a wide viewing angle and excellent visibility over a conventional liquid crystal display device.

The light emission mechanism is said to be as follows: when a voltage is applied between a pair of electrodes with an EL layer including a luminous body provided therebetween, electrons injected from the cathode and holes injected from the anode recombine in the light emission center of the EL layer to form molecular excitons, and energy is released and light is emitted when the molecular excitons return to the ground state. Singlet excitation and triplet excitation are known as excited states, and it is thought that light emission can be achieved through either of the excited states.

An organic compound is mainly used for an EL layer in such a light-emitting element and greatly affects an improvement in the characteristics of the light-emitting element. For this reason, a variety of novel organic compounds have been developed (e.g., Patent Document 1).

### REFERENCE

### Patent Document

### Patent Document 1: Japanese Published Patent Application No. 2011-201869

### SUMMARY OF THE INVENTION

An object of one embodiment of the present invention is to provide a novel organic compound. Another object is to provide a novel organic compound with good thermophysical properties. Another object is to provide a novel organic compound with high current efficiency. Another object is to provide a novel organic compound that can be used in a light-emitting element. Another object is to provide a novel organic compound that can be used in an EL layer of a light-emitting element. Another object is to provide a light-emitting element using a novel organic compound of one embodiment of the present invention. Another object is to provide a light-emitting device, an electronic device, and a lighting device each of which includes a light-emitting element using the novel organic compound of one embodiment of the present invention and has low drive voltage. Another object is to provide a novel light-emitting element, a novel light-emitting device, a novel lighting device, or the like. Note that the descriptions of these objects do not preclude the existence of other objects. In one embodiment of the present invention, there is no need to achieve all the objects. Other objects will be apparent from and can be derived from the description of the specification, the drawings, the claims, and the like.

An organic compound of one embodiment of the present invention includes a π-electron deficient divalent heteroaromatic group and a plurality of sets each including an arylene group and a heterocyclic skeleton that is connected to the heteroaromatic group via the arylene group and includes any of a benzothienocarbazolyl group, a benzofurocarbazolyl group, and an indenocarbazolyl group.

One embodiment of the present invention is an organic compound represented by the following general formula (G1).

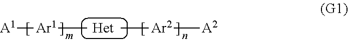

In the general formula (G1), Het represents a substituted or unsubstituted π-electron deficient divalent heteroaromatic group having 2 to 36 carbon atoms, preferably 3 to 36 carbon atoms, further preferably 4 to 36 carbon atoms, and Ar¹ and Ar² each independently represent a substituted or unsubstituted arylene group having 6 to 25 carbon atoms. Further, in and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

According to another embodiment of the present invention, in the above general formula (G1), Het represents a substituted or unsubstituted π-electron deficient divalent hetero aromatic group having 2 to 18 carbon atoms, preferably 3 to 18 carbon atoms, further preferably 4 to 18 carbon atoms, and Ar¹ and Ar² each independently represent a substituted or unsubstituted arylene group having 6 to 25 carbon atoms. Further, in and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

According to another embodiment of the present invention, in the above general formula (G1), Het represents a substituted or unsubstituted π-electron deficient divalent monocyclic nitrogen-containing heteroaromatic group, and Ar¹ and Ar² each independently represent a substituted or unsubstituted arylene group having 6 to 25 carbon atoms. Further, in and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

According to another embodiment of the present invention, in the above general formula (G1), Het represents any of substituted or unsubstituted divalent pyridine, substituted or unsubstituted divalent pyrimidine, substituted or unsubstituted divalent pyrazine, substituted or unsubstituted divalent triazine, substituted or unsubstituted divalent bipyridine, substituted or unsubstituted divalent quinoxaline, and substituted or unsubstituted divalent dibenzoquinoxaline, and Ar¹ and Ar² each independently represent a substituted or unsubstituted arylene group having 6 to 25 carbon atoms. Further, in and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

Another embodiment of the present invention is an organic compound represented by the following general formula (G2).

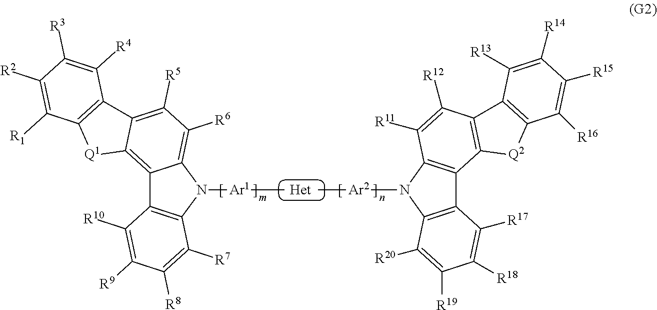

In the general foimula (G2), Het represents any of substituted or unsubstituted divalent pyridine, substituted or unsubstituted divalent pyrimidine, substituted or unsubstituted divalent pyrazine, substituted or unsubstituted divalent triazine, substituted or unsubstituted divalent bipyridine, substituted or unsubstituted divalent quinoxaline, and substituted or unsubstituted divalent dibenzoquinoxaline, and Ar¹ and Ar² each independently represent a substituted or unsubstituted arylene group having 6 to 25 carbon atoms. Further, in and n each independently represent a natural number greater than or equal to 1 and less than or equal to 5. Further, Q¹ and Q² each independently represent any of a sulfur atom, an oxygen atom, and a substituted or unsubstituted carbon atom, and R¹ to R²⁰ each independently represent any of hydrogen, an alkyl group having 1 to 6 carbon atoms, and an aryl group having 6 to 13 carbon atoms.

A further embodiment of the present invention is an organic compound represented by the following general formula (G3).

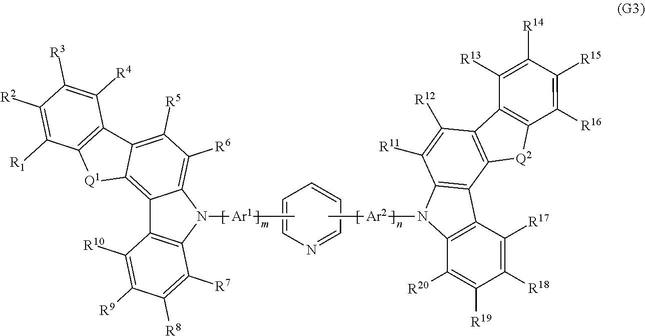

In the general formula (G3), Ar¹ and Ar² each independently represent a substituted or unsubstituted arylene group having 6 to 25 carbon atoms. Further, in and n each independently represent a natural number greater than or equal to 1 and less than or equal to 5. Further, Q¹ and Q² each independently represent any of a sulfur atom, an oxygen atom, and a substituted or unsubstituted carbon atom, and R¹ to R²⁰ each independently represent any of hydrogen, an alkyl group having 1 to 6 carbon atoms, and an aryl group having 6 to 13 carbon atoms.

A still further embodiment of the present invention is an organic compound represented by the following general formula (G4).

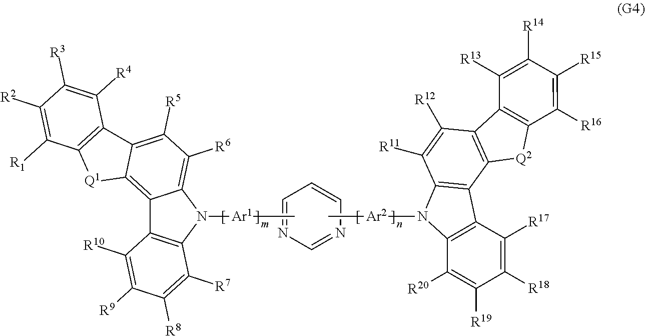

In the general formula (G4), Ar¹ and Ar² each independently represent a substituted or unsubstituted arylene group having 6 to 25 carbon atoms. Further, in and n each independently represent a natural number greater than or equal to 1 and less than or equal to 5. Further, Q¹ and Q² each independently represent any of a sulfur atom, an oxygen atom, and a substituted or unsubstituted carbon atom, and R¹ to R²⁰ each independently represent any of hydrogen, an alkyl group having 1 to 6 carbon atoms, and an aryl group having 6 to 13 carbon atoms.

A yet still further embodiment of the present invention is an organic compound represented by the following structural formula (100).

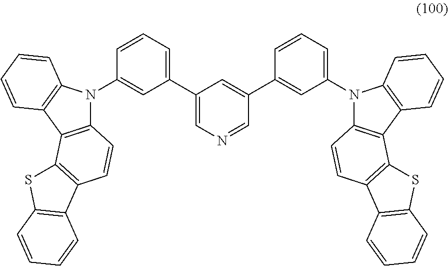 [SMILES: c1cc(-c2cncc(-c3cccc(-n4c5ccccc5c5c6sc7ccccc7c6ccc54)c3)c2)cc(-n2c3ccccc3c3c4sc5ccccc5c4ccc32)c1]

Another embodiment of the present invention is an organic compound represented by the following structural formula (200).

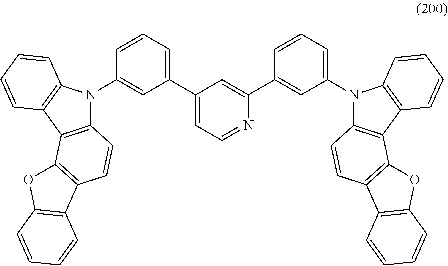 [SMILES: c1cc(-c2ccnc(-c3cccc(-n4c5ccccc5c5c6oc7ccccc7c6ccc54)c3)c2)cc(-n2c3ccccc3c3c4oc5ccccc5c4ccc32)c1]

Another embodiment of the present invention is an organic compound represented by the following structural formula (115).

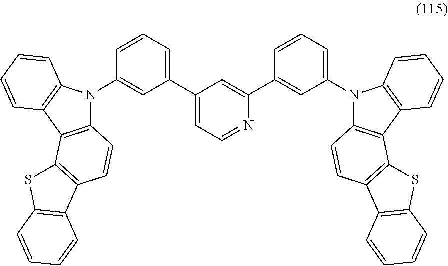 [SMILES: c1cc(-c2ccnc(-c3cccc(-n4c5ccccc5c5c6sc7ccccc7c6ccc54)c3)c2)cc(-n2c3ccccc3c3c4sc5ccccc5c4ccc32)c1]

Another embodiment of the present invention is an organic compound represented by the following structural formula (112).

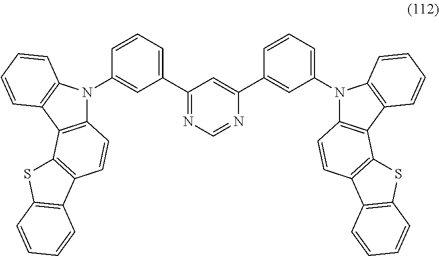 [SMILES: c1cc(-c2cc(-c3cccc(-n4c5ccccc5c5c6sc7ccccc7c6ccc54)c3)ncn2)cc(-n2c3ccccc3c3c4sc5ccccc5c4ccc32)c1]

Another embodiment of the present invention is a light-emitting element including the organic compound in any of the above structures.

Another embodiment of the present invention is a light-emitting device including the light-emitting element in the above structure and a transistor or a substrate.

Note that one embodiment of the present invention includes not only a light-emitting device including the light-emitting element but also an electronic device and a lighting device each including the light-emitting device.

Thus, another embodiment of the present invention is an electronic device including the above-described light-emitting device and a microphone, a camera, a button for operation, an external connection portion, or a speaker. Another embodiment of the present invention is an electronic device including the above-described light-emitting device and a housing, a cover, or a support.

The light-emitting device in this specification refers to an image display device and a light source (e.g., a lighting device). In addition, the light-emitting device includes, in its category, all of a module in which a light-emitting device is connected to a connector such as a flexible printed circuit (FPC), a tape carrier package (TCP), a module in which a printed wiring board is provided on the tip of a TCP, and a module in which an integrated circuit (IC) is directly mounted on a light-emitting element by a chip on glass (COG) method.

According to one embodiment of the present invention, a novel organic compound can be provided. A novel organic compound with good thermophysical properties can be provided. A novel organic compound with high current efficiency can be provided. A novel organic compound that can be used in a light-emitting element can be provided. A novel organic compound that can be used in an EL layer of a light-emitting element can be provided: A light-emitting element using a novel organic compound of one embodiment of the present invention can be provided. A light-emitting device, an electronic device, and a lighting device, each of which includes a light-emitting element using the novel organic compound of one embodiment of the present invention and has low drive voltage and high reliability, can be provided. A novel light-emitting element, a novel light-emitting device, a novel lighting device, or the like can be provided. Note that the description of these effects does not preclude the existence of other effects. One embodiment of the present invention does not necessarily achieve all the effects listed above. Other effects will be apparent from and can be derived from the description of the specification, the drawings, the claims, and the like.

### BRIEF DESCRIPTION OF THE DRAWINGS

In the accompanying drawings:

FIG. 1 illustrates a structure of a light-emitting element;

FIGS. 2A and 2B each illustrate a structure of a light-emitting element;

FIGS. 3A to 3C illustrate a light-emitting device;

FIGS. 4A, 4B, 4C, 4D, 4D′1, and 4D′2 illustrate electronic devices;

FIGS. 5A to 5C illustrate an electronic device;

FIG. 6 illustrates lighting devices;

FIGS. 7A and 7B show ¹H-NMR charts of the organic compound represented by the structural formula (100);

FIGS. 8A and 8B show ultraviolet-visible absorption spectra and emission spectra of the organic compound represented by the structural formula (100);

FIG. 9 shows results of LC-MS measurements of the organic compound represented by the structural formula (100);

FIGS. 10A and 10B show ¹H-NMR charts of the organic compound represented by the structural formula (200);

FIGS. 11A and 11B show ultraviolet-visible absorption spectra and emission spectra of the organic compound represented by the structural formula (200);

FIG. 12 shows results of LC-MS measurements of the organic compound represented by the structural formula (200);

FIGS. 13A and 13B show ¹H-NMR charts of the organic compound represented by the structural formula (115);

FIGS. 14A and 14B show ultraviolet-visible absorption spectra and emission spectra of the organic compound represented by the structural formula (115);

FIG. 15 shows results of LC-MS measurements of the organic compound represented by the structural formula (115);

FIG. 16 illustrates a structure of a light-emitting element;

FIG. 17 shows luminance-current efficiency characteristics of a light-emitting element 1;

FIG. 18 shows voltage-current characteristics of the light-emitting element 1;

FIG. 19 shows luminance-chromaticity characteristics of the light-emitting element 1;

FIG. 20 shows luminance-power efficiency characteristics of the light-emitting element 1;

FIG. 21 shows luminance-external quantum efficiency characteristics of the light-emitting element 1;

FIG. 22 shows an emission spectrum of the light-emitting element 1;

FIG. 23 shows reliability of the light-emitting element 1;

FIG. 24 shows luminance-current efficiency characteristics of a light-emitting element 2;

FIG. 25 shows voltage-current characteristics of the light-emitting element 2;

FIG. 26 shows luminance-chromaticity characteristics of the light-emitting element 2;

FIG. 27 shows luminance-power efficiency characteristics of the light-emitting element 2;

FIG. 28 shows luminance-external quantum efficiency characteristics of the light-emitting element 2;

FIG. 29 shows an emission spectrum of the light-emitting element 2;

FIG. 30 shows reliability of the light-emitting element 2;

FIGS. 31A to 31E illustrate a lighting device; and

FIGS. 32A and 32B show ¹H-NMR charts of the organic compound represented by the structural formula (112).

### DETAILED DESCRIPTION OF THE INVENTION

Hereinafter, embodiments of the present invention will be described with reference to the accompanying drawings. Note that the present invention is not limited to the following description, and various changes and modifications can be made without departing from the spirit and scope of the present invention. Therefore, the present invention should not be construed as being limited to the description in the following embodiments.

Note that the terms “film” and “layer” can be interchanged with each other depending on the case or circumstances. For example, the term “conductive layer” can be changed into the term “conductive film” in some cases. Also, the term “insulating film” can be changed into the term “insulating layer” in some cases.

### Embodiment 1

In this embodiment, an organic compound of one embodiment of the present invention is described. The organic compound of one embodiment of the present invention includes a π-electron deficient divalent heteroaromatic group and a plurality of sets each including an arylene group and a heterocyclic skeleton connected to the heteroaromatic group via the arylene group.

One embodiment of the present invention is an organic compound represented by the following general formula (G1).

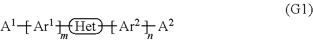

In the general formula (G1), Het represents a π-electron deficient divalent heteroaromatic group, which is specifically a substituted or unsubstituted π-electron deficient divalent heteroaromatic group having 2 to 36 carbon atoms, preferably 3 to 36 carbon atoms, further preferably 4 to 36 carbon atoms, and Ar¹ and Ar² each independently represent an arylene group, which is specifically a substituted or unsubstituted arylene group having 6 to 25 carbon atoms. Further, in and n each independently represent a natural number greater than or equal to 1 and less than or equal to 5, and A¹ and A² each independently represent a heterocyclic skeleton, which is specifically any of a substituted or unsubstituted benzothienocarbazolyl group, a substituted or unsubstituted benzofurocarbazolyl group, and a substituted or unsubstituted indenocarbazolyl group.

The molecular weight of the compound represented by the general formula (G1) is preferably 700 or more in terms of thermophysical properties.

The molecular weight of the compound represented by the general formula (G1) is preferably 1500 or less in terms of sublimation properties.

According to another embodiment of the present invention, in the above general formula (G1), Het represents a substituted or unsubstituted π-electron deficient divalent heteroaromatic group having 2 to 18 carbon atoms, preferably 3 to 18 carbon atoms, further preferably 4 to 18 carbon atoms, and Ar¹ and Ar² each independently represent a substituted or unsubstituted atylene group having 6 to 25 carbon atoms. Further, in and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

The π-electron deficient divalent heteroaromatic group refers to a heteroaromatic group which includes two or more five-membered heteroaromatic rings including nitrogen and carbon or one or more six-membered heteroaromatic rings including nitrogen and carbon, and in which two hydrogen atoms are removed from the heteroaromatic rings. Specific examples of the π-electron deficient divalent heteroaromatic group are divalent pyridine, divalent pyrimidine, divalent pyrazine, divalent triazine, divalent bipyridine, divalent quinoxaline, divalent imidazole, divalent triazole, and the like.

According to another embodiment of the present invention, in the above general formula (G1), Het represents a substituted or unsubstituted π-electron deficient divalent monocyclic nitrogen-containing heteroaromatic group, and Ar¹ and Ar² each independently represent a substituted or unsubstituted arylene group having 6 to 25 carbon atoms. Further, 171 and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

Specific examples of the above π-electron deficient divalent monocyclic nitrogen-containing heteroaromatic group are divalent pyridine, divalent pyrimidine, divalent triazine, and the like.

According to another embodiment of the present invention, in the above general formula (G1), Het represents any of substituted or unsubstituted divalent pyridine, substituted or unsubstituted divalent pyrimidine, substituted or unsubstituted divalent pyrazine, substituted or unsubstituted divalent triazine, substituted or unsubstituted divalent bipyridine, substituted or unsubstituted divalent quinoxaline, and substituted or unsubstituted divalent dibenzoquinoxaline, and Ar¹ and Ar² each independently represent a substituted or unsubstituted arylene group having 6 to 25 carbon atoms. Further, in and n each independently represent a natural number greater than or equal to 1 and less than or equal to 5, and A¹ and A² each independently represent any of a substituted or unsubstituted benzothienocarbazolyl group, a substituted or unsubstituted benzofurocarbazolyl group, and a substituted or unsubstituted indenocarbazolyl group.

Another embodiment of the present invention is an organic compound represented by the following general formula (G2).

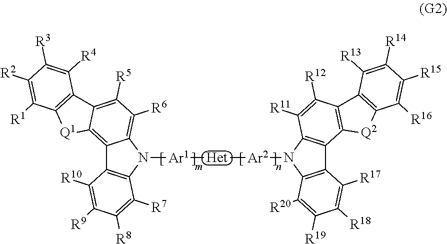

In the general formula (G2), Het represents any of substituted or unsubstituted divalent pyridine, substituted or unsubstituted divalent pyrimidine, substituted or unsubstituted divalent pyrazine, substituted or unsubstituted divalent triazine, substituted or unsubstituted divalent bipyridine, substituted or unsubstituted divalent quinoxaline, and substituted or unsubstituted divalent dibenzoquinoxaline, and Ar¹ and Ar² each independently represent a substituted or unsubstituted arylene group having 6 to 25 carbon atoms. Further, in and n each independently represent a natural number greater than or equal to 1 and less than or equal to 5. Further, Q¹ and Q² each independently represent any of a sulfur atom, an oxygen atom, and a substituted or unsubstituted carbon atom, and R¹ to R²⁰ each independently represent any of hydrogen, an alkyl group having 1 to 6 carbon atoms, and an aryl group having 6 to 13 carbon atoms.

A further embodiment of the present invention is an organic compound represented by the following general formula (G3).

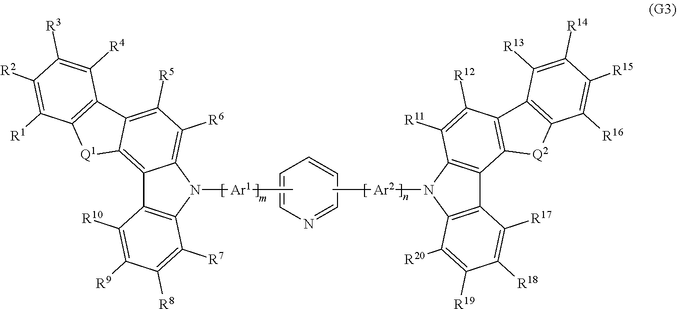

In the general formula (G3), Ar¹ and Ar² each independently represent a substituted or unsubstituted arylene group having 6 to 25 carbon atoms. Further, in and n each independently represent a natural number greater than or equal to 1 and less than or equal to 5. Further, Q¹ and Q² each independently represent any of a sulfur atom, an oxygen atom, and a substituted or unsubstituted carbon atom, and R¹ to R²⁰ each independently represent any of hydrogen, an alkyl group having 1 to 6 carbon atoms, and an aryl group having 6 to 13 carbon atoms.

A still further embodiment of the present invention is an organic compound represented by the following general formula (G4).

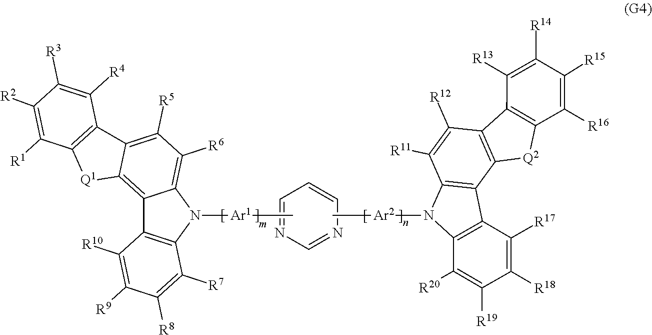

In the general formula (G4), Ar¹ and Ar² each independently represent a substituted or unsubstituted arylene group having 6 to 25 carbon atoms. Further, in and n each independently represent a natural number greater than or equal to 1 and less than or equal to 5. Further, Q¹ and Q² each independently represent any of a sulfur atom, an oxygen atom, and a substituted or unsubstituted carbon atom, and R¹ to R²⁰ each independently represent any of hydrogen, an alkyl group having 1 to 6 carbon atoms, and an aryl group having 6 to 13 carbon atoms.

In the above general formulae (G1) to (G3), Ar¹ and Ar² each independently represent a substituted or unsubstituted arylene group having 6 to 25 carbon atoms. When Het represents divalent pyridine in the general formulae (G1) and (G2), bonding positions are preferably the 3- and 5-positions. Such a molecular structure allows the compound to have a higher electron-transport property than the structure in which the bonding positions are the 2- and 6-positions. This leads to higher current efficiency. Also in the general formula (G3), the bonding positions in divalent pyridine are preferably the 3- and 5-positions, in which case steric hindrance around a nitrogen atom in pyridine is reduced. Such a structure allows the compound to have a higher electron-transport property than the structure in which the bonding positions are the 1- and 6-positions. This leads to higher current efficiency.

When Q¹ and Q² in the above general formulae (G2) to (G4) each independently represent a carbon atom having a substituent, the substituent is preferably one or two alkyl groups each having 1 to 6 carbon atoms, one or two phenyl groups, or one or two biphenyl groups.

When the arylene groups represented by Ar¹ and Ar² in the above general formulae (G1) to (G4) are each independently a phenylene group, meta-phenylene is preferable to para-phenylene as the phenylene group. This is because the π-orbital interactions between substituents bonded to Ar¹ and between substituents bonded to Ar² can be reduced and the band gap width or the phosphorescent level can be increased. Alternatively, when the arylene groups are each independently a biphenyldiyl group, a 1,1′-biphenyl-3,3′-diyl group is preferable.

In the above general formulae (G1) to (G4), Ar¹ and Ar² each independently represent a substituted or unsubstituted arylene group having 6 to 25 carbon atoms, and examples thereof are a phenylene group, a biphenylene group, and the like. Specific examples are a 1,2-phenylene group, a 1,3-phenylene group, a 1,4-phenylene group, a 2,6-toluylene group, a 3,5-toluylene group, a 2,4-toluylene group, a 4,6-dimethylbenzene-1,3-diyl group, a 2,4,6-trimethylbenzene-1,3-diyl group, a 2,3,5,6-tetramethylbenzene-1,4-diyl group, a 3,3’-biphenylene group, a 3,4′-biphenylene group, a 4,4′-biphenylene group, a 1,1′:3′,1″-terbenzene-3,3″-diyl group, a 1,1′:4′,1″-terbenzene-3,3″-diyl group, a 1,1′:4′,1″-terbenzene-4,4″-diyl group, a 1,4-naphthylene group, a 1,5-naphthylene group, a 2,6-naphthylene group, a 2,7-naphthylene group, a 2,7-fluorenylene group, a 9,9-dimethyl-2,7-fluorenylene group, a 9,9-diphenyl-2,7-fluorenylene group, a 9,9-dimethyl-1,4-fluorenylene group, a spiro-9,9t-bifluorene-2,7-diyl group, a 9,10-dihydro-2,7-phenanthrylene group, a 2,7-phenanthrylene group, a 3,6-phenanthrylene group, a 9,10-phenanthrylene group, and the like. Note that there is no limitation on the bonding position.

Specific examples of the alkyl group having 1 to 6 carbon atoms in the above general formulae (G2) to (G4) are a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, a 2,3-dimethylbutyl group, and the like.

Specific examples of the aryl group having 6 to 12 carbon atoms in the above general formulae (G2) to (G4) are a phenyl group, a naphthyl group, a biphenyl group, and the like. Note that there is no limitation on the bonding position.

Next, as an example of a method of synthesizing the organic compound of one embodiment of the present invention, an example of a method of synthesizing the organic compound represented by the above general formula (G1) is described.

### <<Method of Synthesizing Organic Compound Represented by General Formula (G1)>>

The organic compound represented by the general foimula (G1) can be synthesized by a simple synthesis scheme, as shown below. For example, as shown in the following scheme (a-1), a compound 1, a compound 2, and a compound 3 are coupled by the Suzuki-Miyaura reaction to give the organic compound.

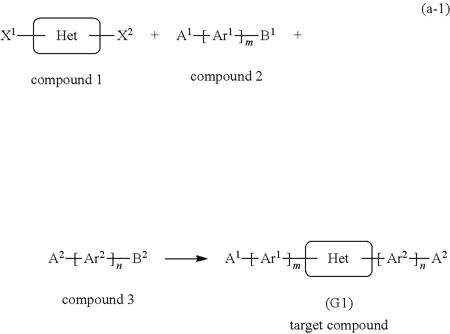

In the above synthesis scheme (a-1), Het represents a substituted or unsubstituted π-electron deficient divalent heteroaromatic group, and X¹ and X² each independently represent a halogen or a triflate group. Further, A¹ and A² each independently represent any of a substituted or unsubstituted benzothienocarbazolyl group, a substituted or unsubstituted benzofurocarbazolyl group, and a substituted or unsubstituted indenocarbazolyl group, and B¹ and B² each independently represent boronic acid or boronic acid ester. Further, Ar¹ and Ar² each independently represent a substituted or unsubstituted arylene group having 6 to 25 carbon atoms, and m and n each independently represent a natural number greater than or equal to 1 and less than or equal to 5.

In the above synthesis scheme (a-1), when the compound 2 and the compound 3 are different compounds, the compound 1 and the compound 2 are coupled and then the resulting substance and the compound 3 are coupled, in which case the object of the synthesis can be obtained with high purity in a high yield.

Non-limiting examples of the palladium catalyst that can be used in the synthesis scheme (a-1) are palladium(II) acetate, tetrakis(triphenylphosphine)palladium(0), bis(triphenylphosphine)palladium(II) dichloride, and the like. Non-limiting examples of a ligand of the palladium catalyst that can be used in the synthesis scheme (a-1) are tri(ortho-tolyl)phosphine, triphenylphosphine, tricyclohexylphosphine, and the like.

Non-limiting examples of the base that can be used in the synthesis scheme (a-1) are an organic base such as sodium tert-butoxide, an inorganic base such as potassium carbonate or sodium carbonate, and the like. Non-limiting examples of the solvent that can be used are a mixed solvent of toluene and water, a mixed solvent of toluene, an alcohol such as ethanol, and water, a mixed solvent of xylene and water, a mixed solvent of xylene, an alcohol such as ethanol, and water, a mixed solvent of benzene and water, a mixed solvent of benzene, an alcohol such as ethanol, and water, a mixed solvent of an ether such as ethylene glycol dimethyl ether and water, a mixed solvent of an ether such as ethylene glycol dimethyl ether, an alcohol such as t-butyl alcohol, and water, and the like.

In the above synthesis scheme (a-1), the compound 2 and the compound 3 are organic boron compounds and hence the Suzuki-Miyaura coupling reaction is employed to cause a reaction. Alternatively, in the case where the compound 2 and the compound 3 are organic aluminum compounds, organic zirconium compounds, organozinc compounds, organic tin compounds, or the like, a cross coupling reaction can be employed to give the object of the synthesis. However, the present invention is not limited thereto.

Alternatively, in the synthesis scheme (a-1), a reaction may be caused using a diboronic acid compound as the compound 1 (in which X¹ and X² each independently represent boronic acid or boronic acid ester) and halogen compounds or triflate compounds as the compound 2 and the compound 3 (in which B¹ and B² each independently represent a halogen or a triflate group).

The above is the description of the example of a method of synthesizing the organic compound as one embodiment of the present invention; however, the present invention is not limited thereto and another synthesis method may be employed.

Shown below are the specific structural formulae of the organic compound (general formula (G1)) as one embodiment of the present invention (the following structural formulae (100) to (135), (200) to (208), and (300) to (303)). Note that one embodiment of the present invention is not limited thereto.

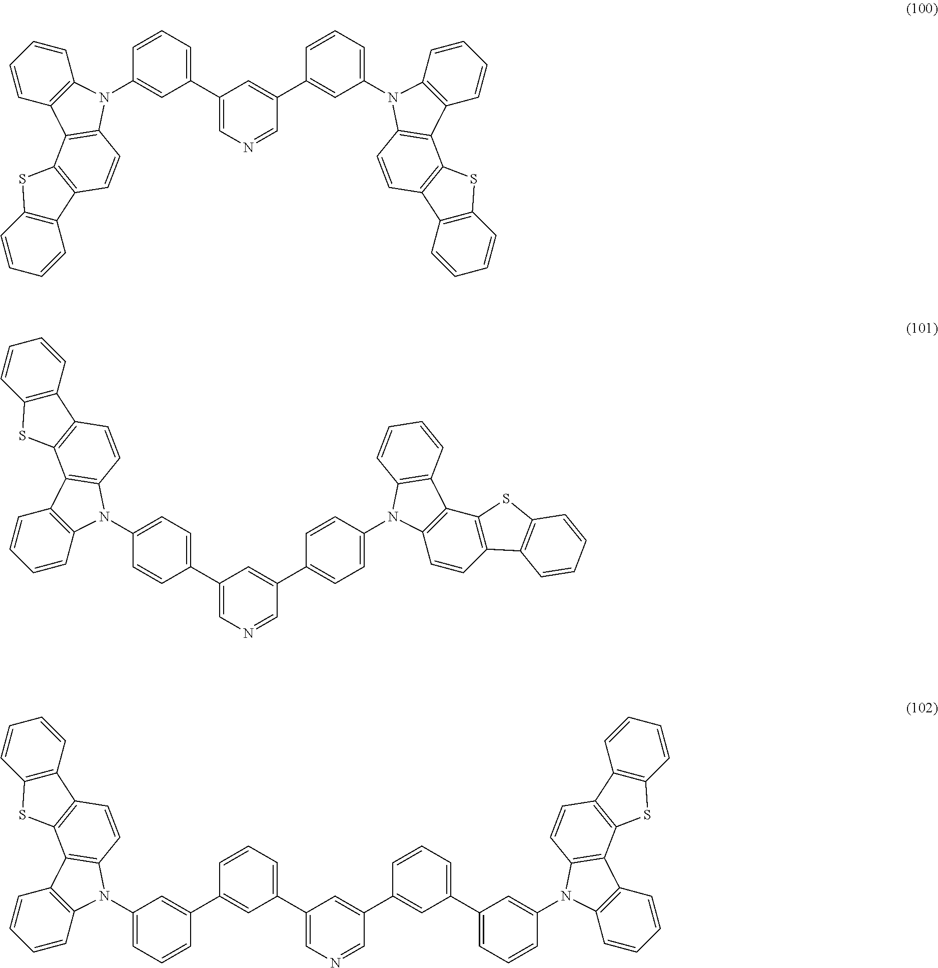

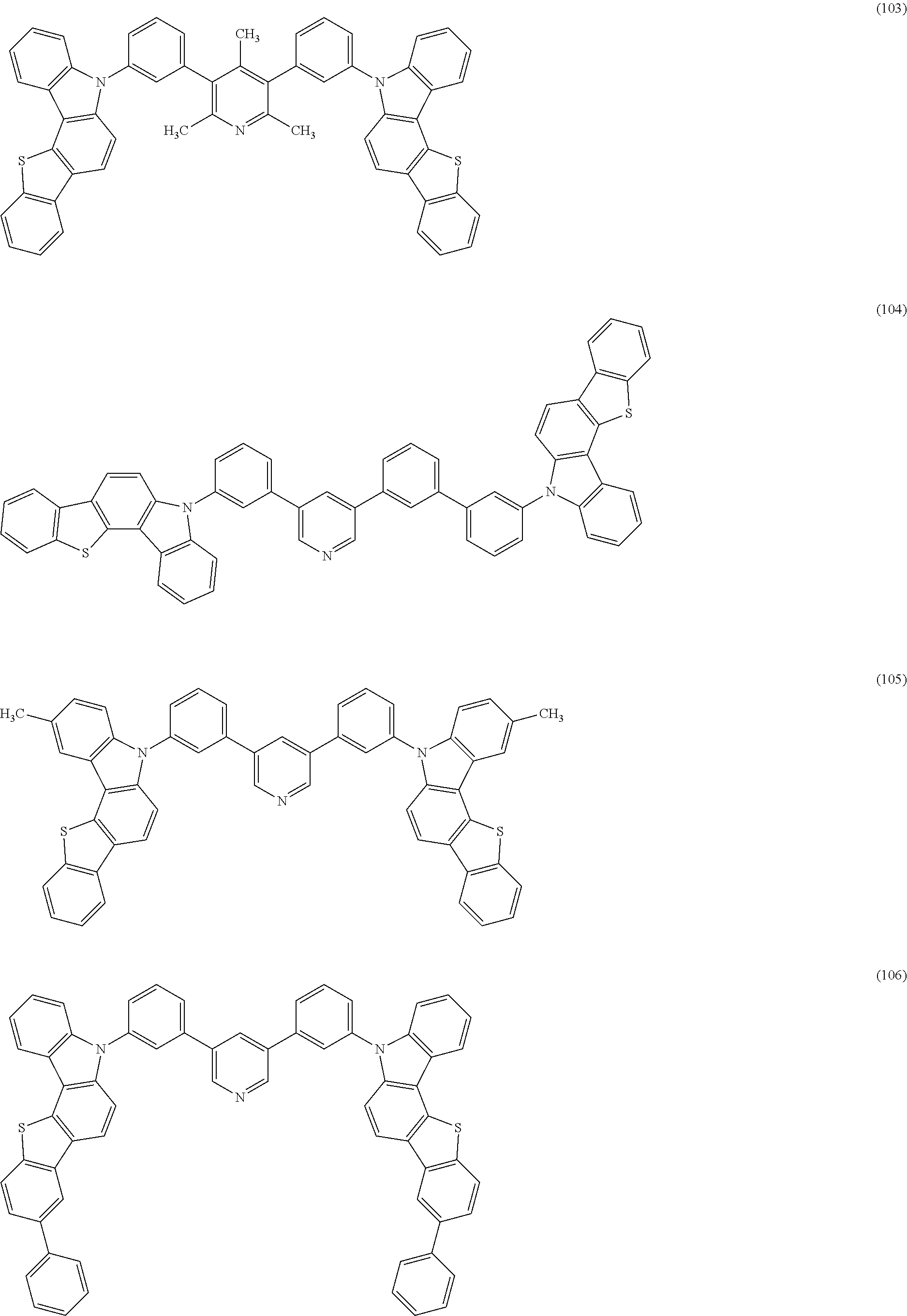

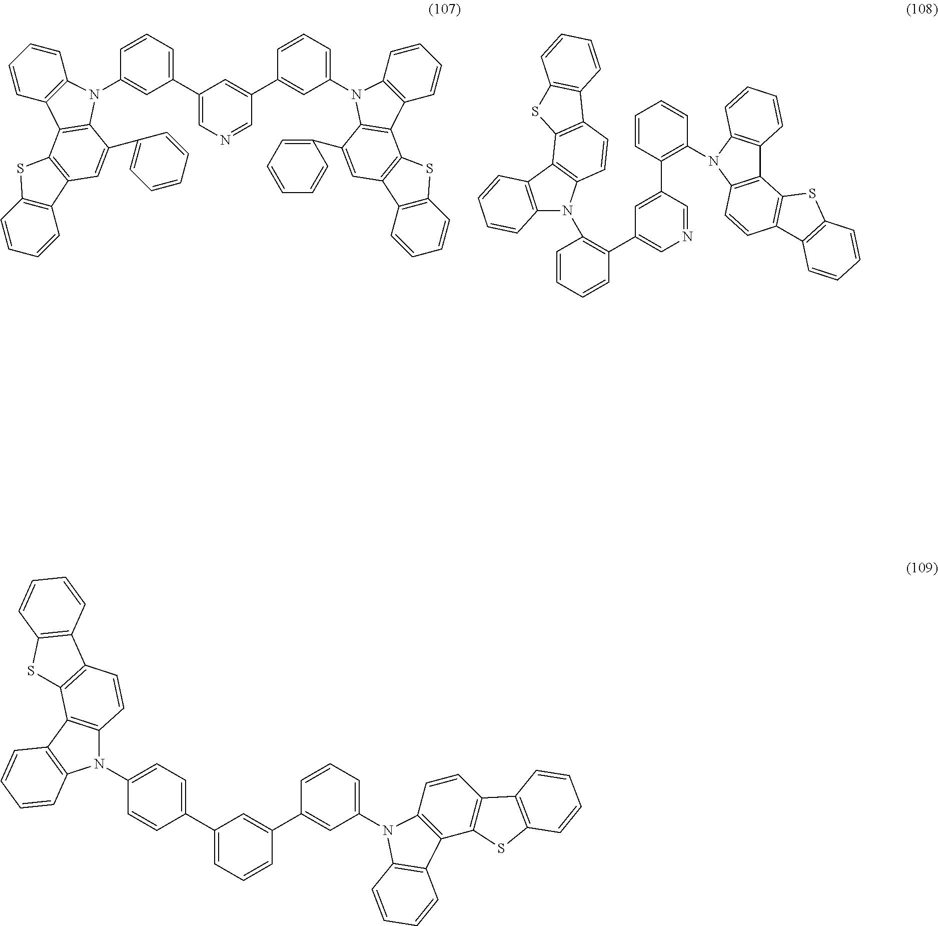

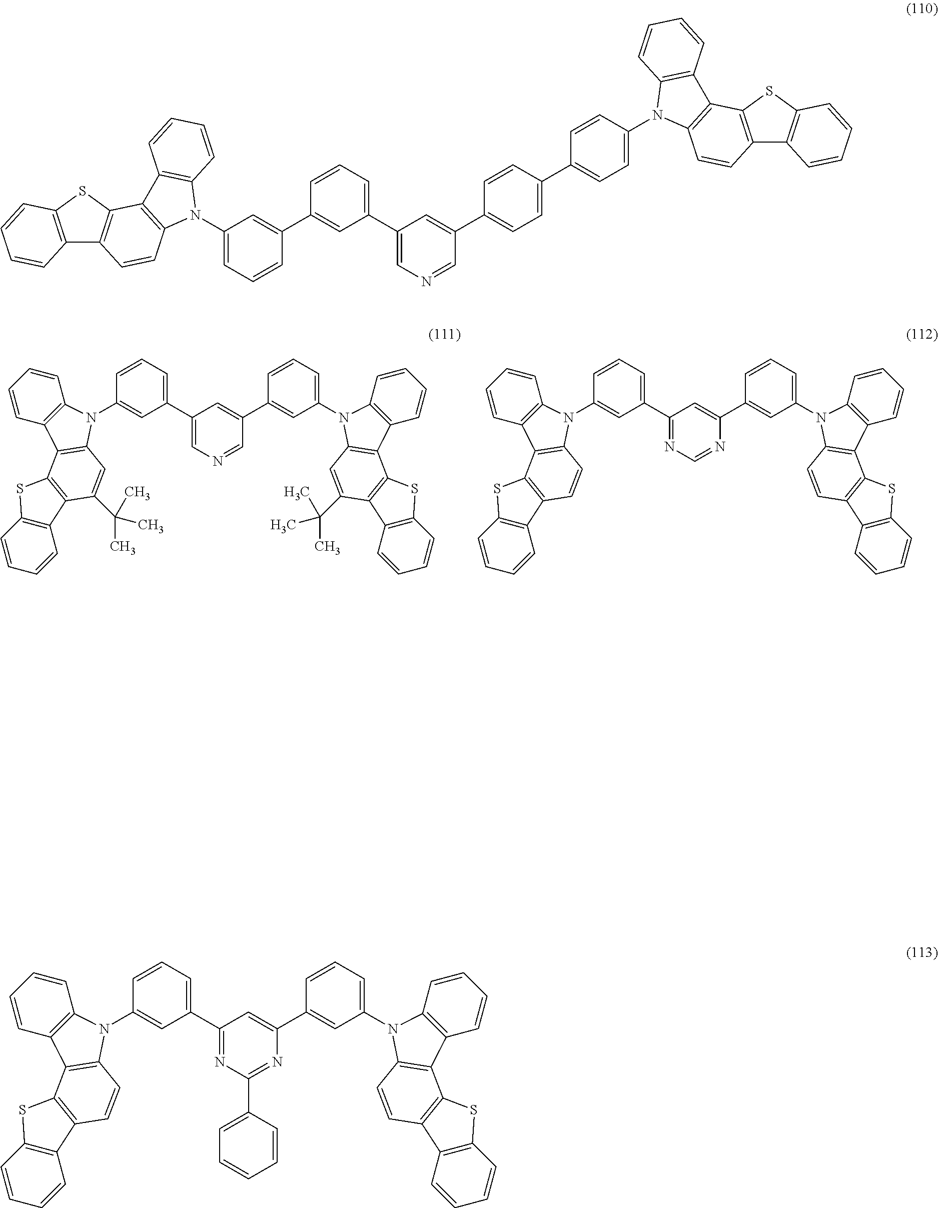

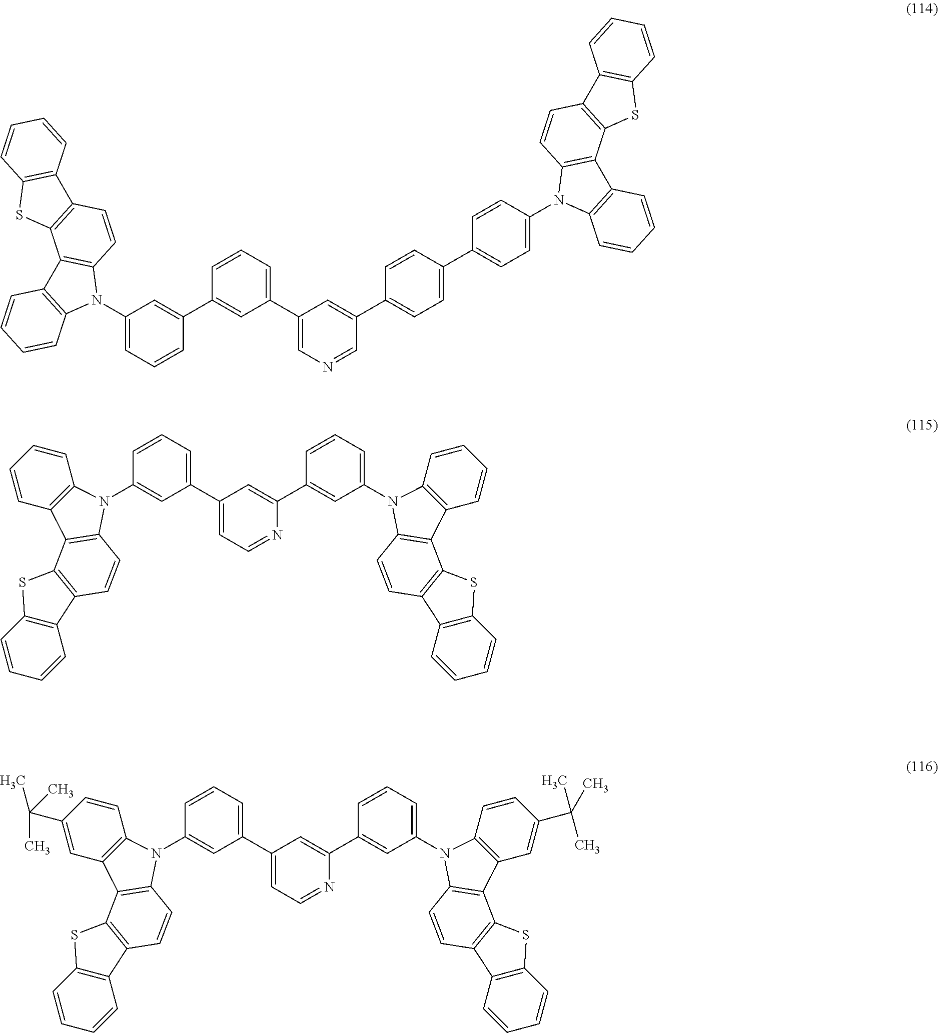

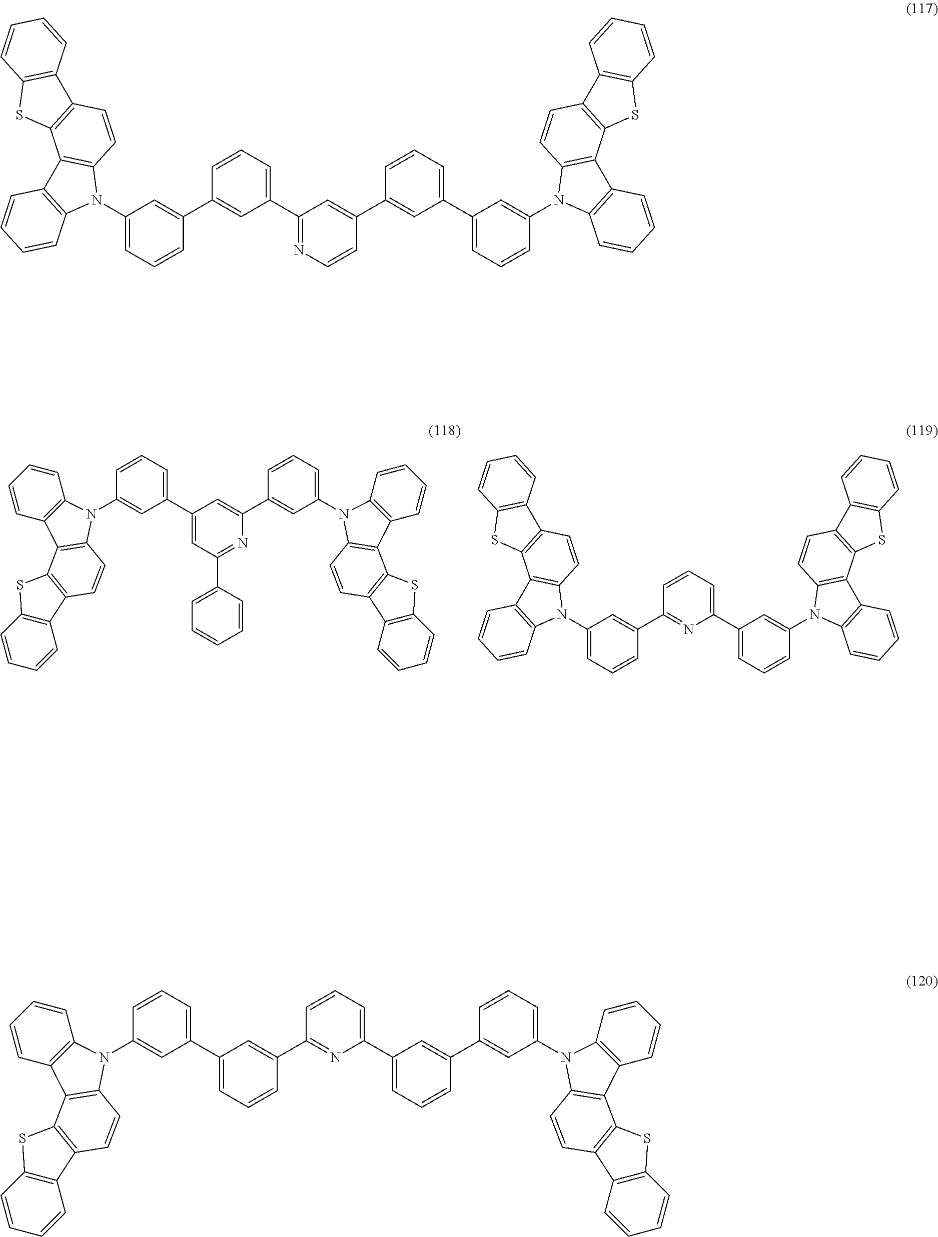

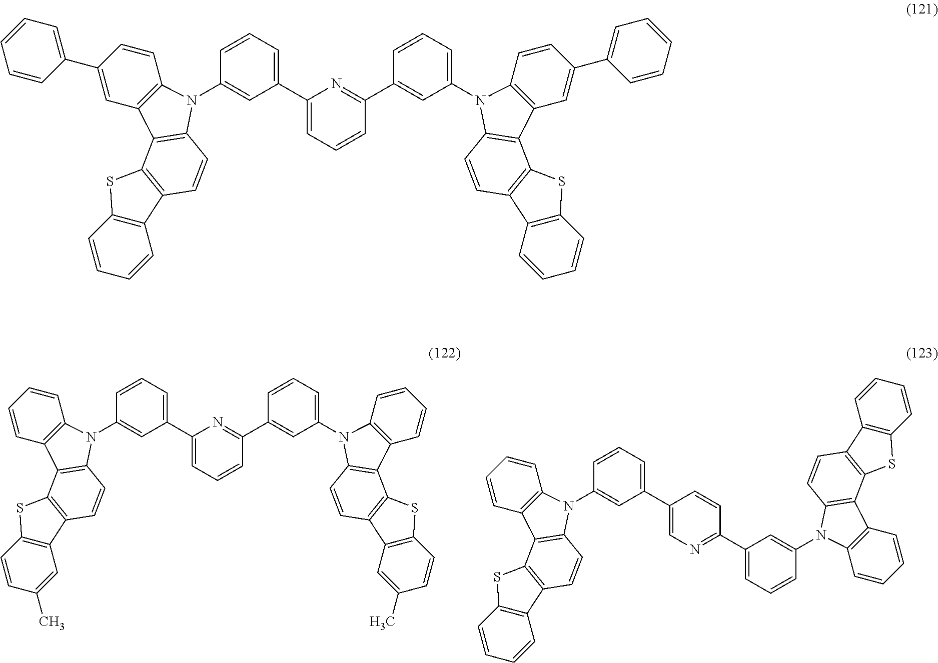

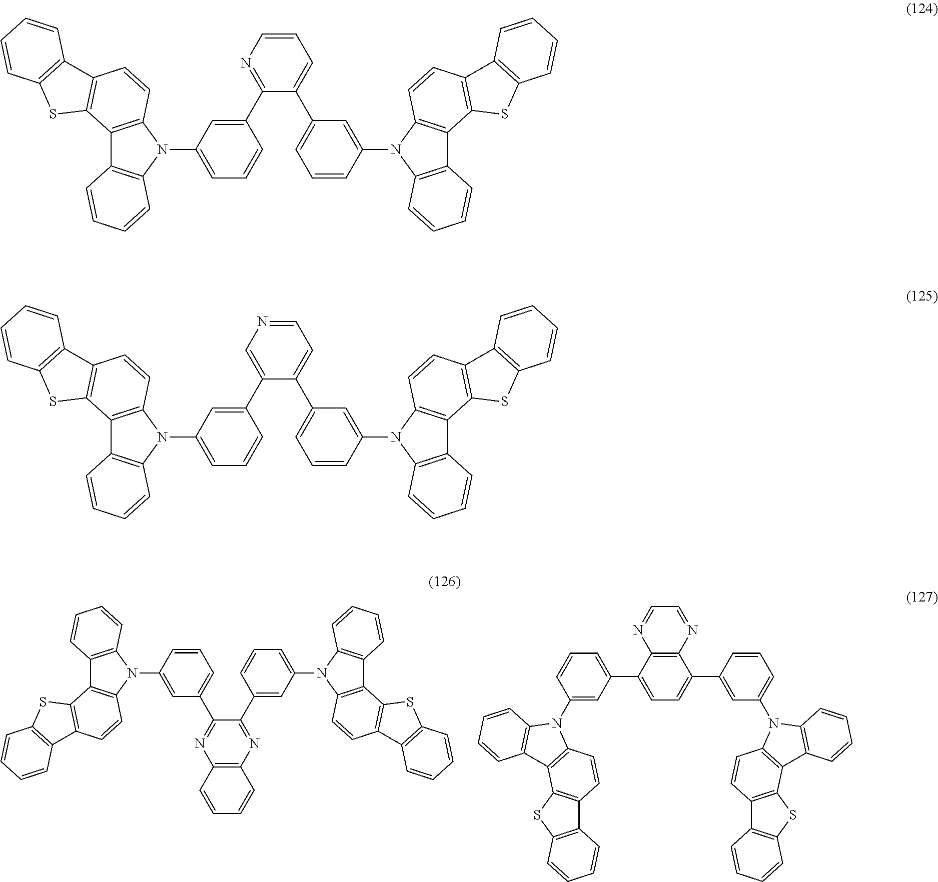

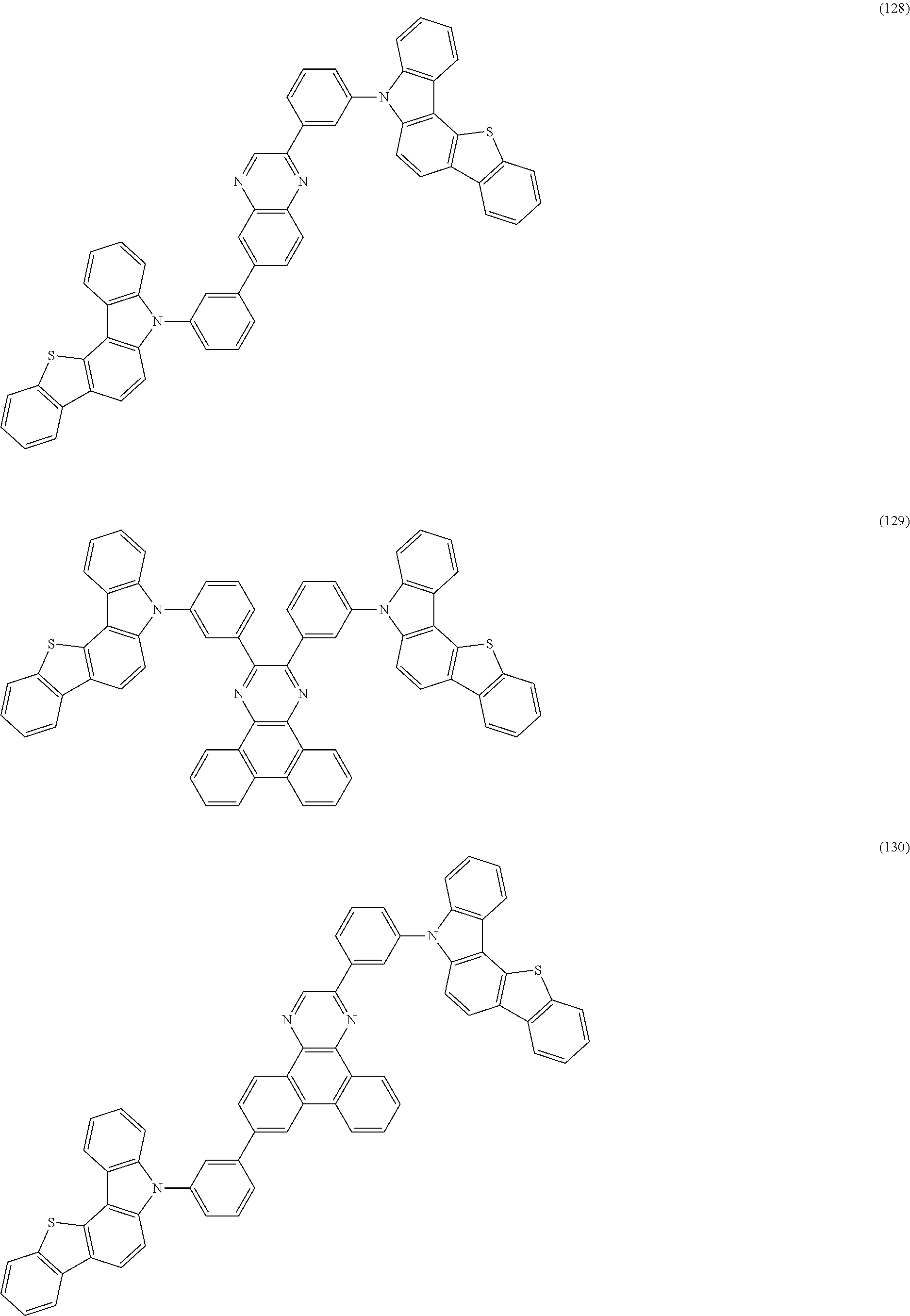

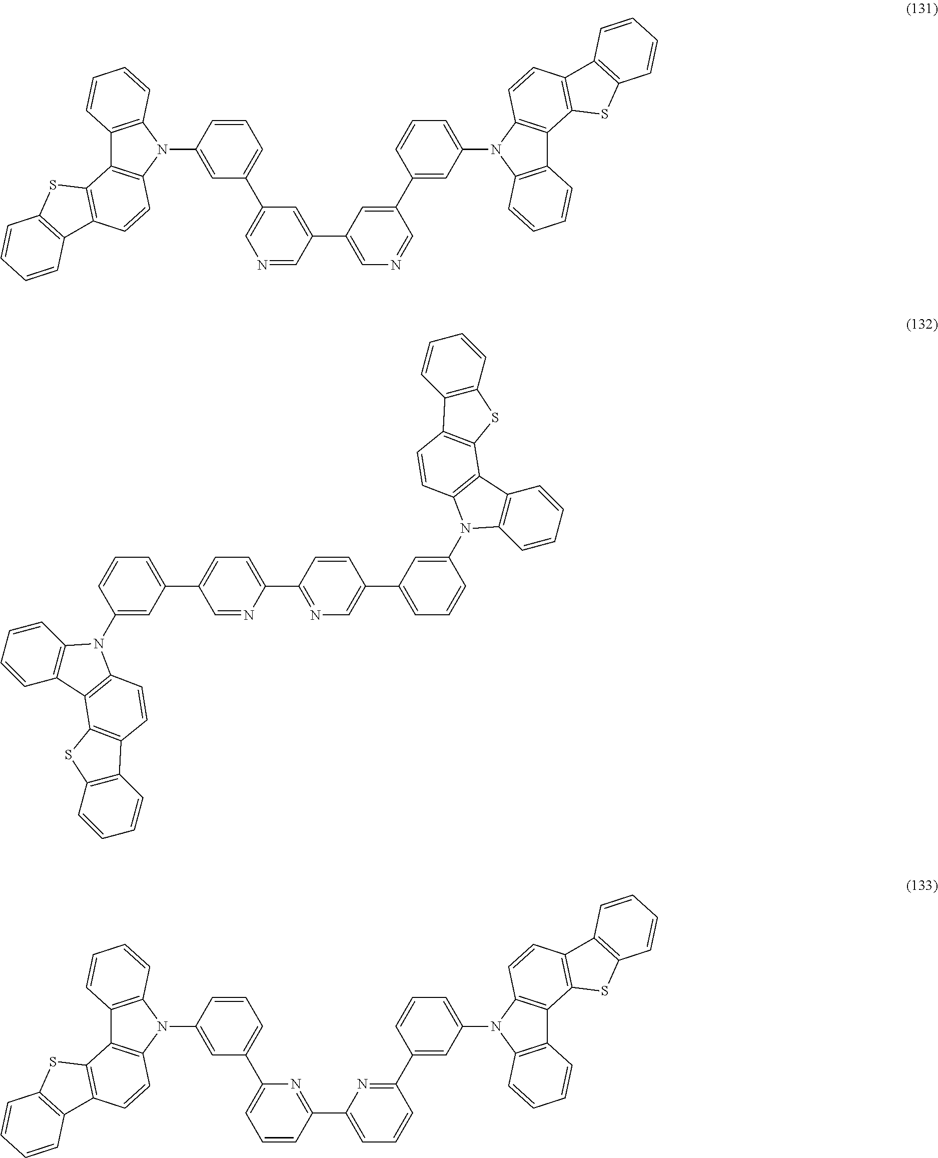

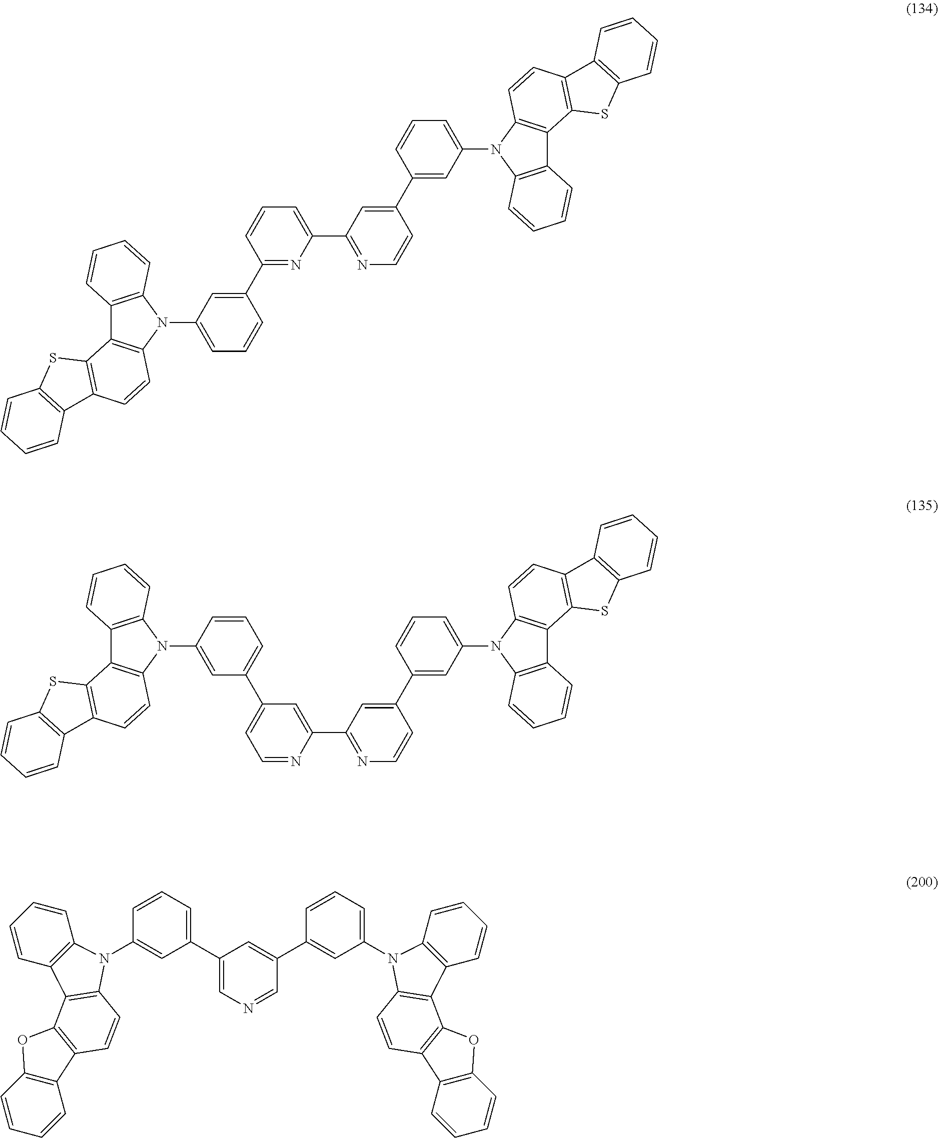

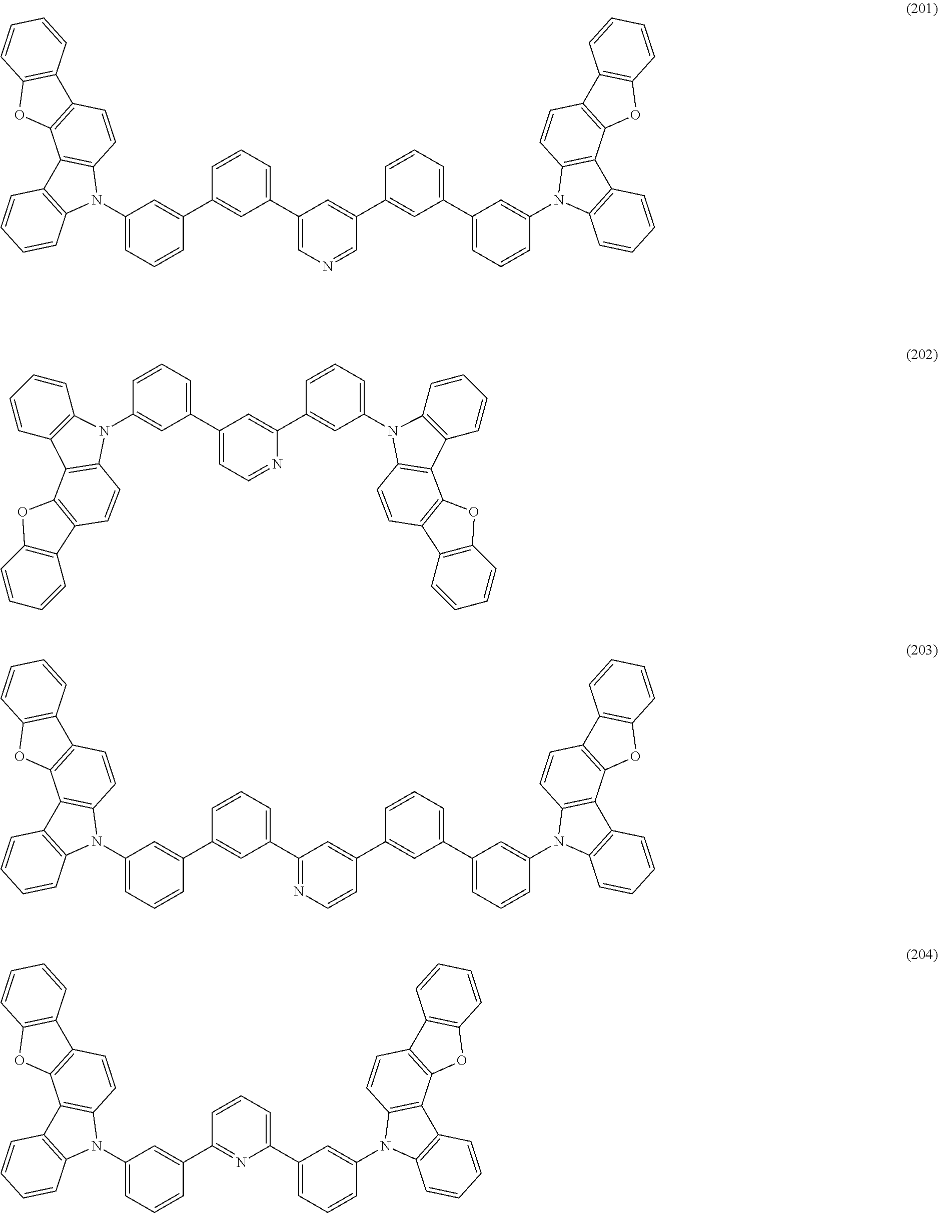

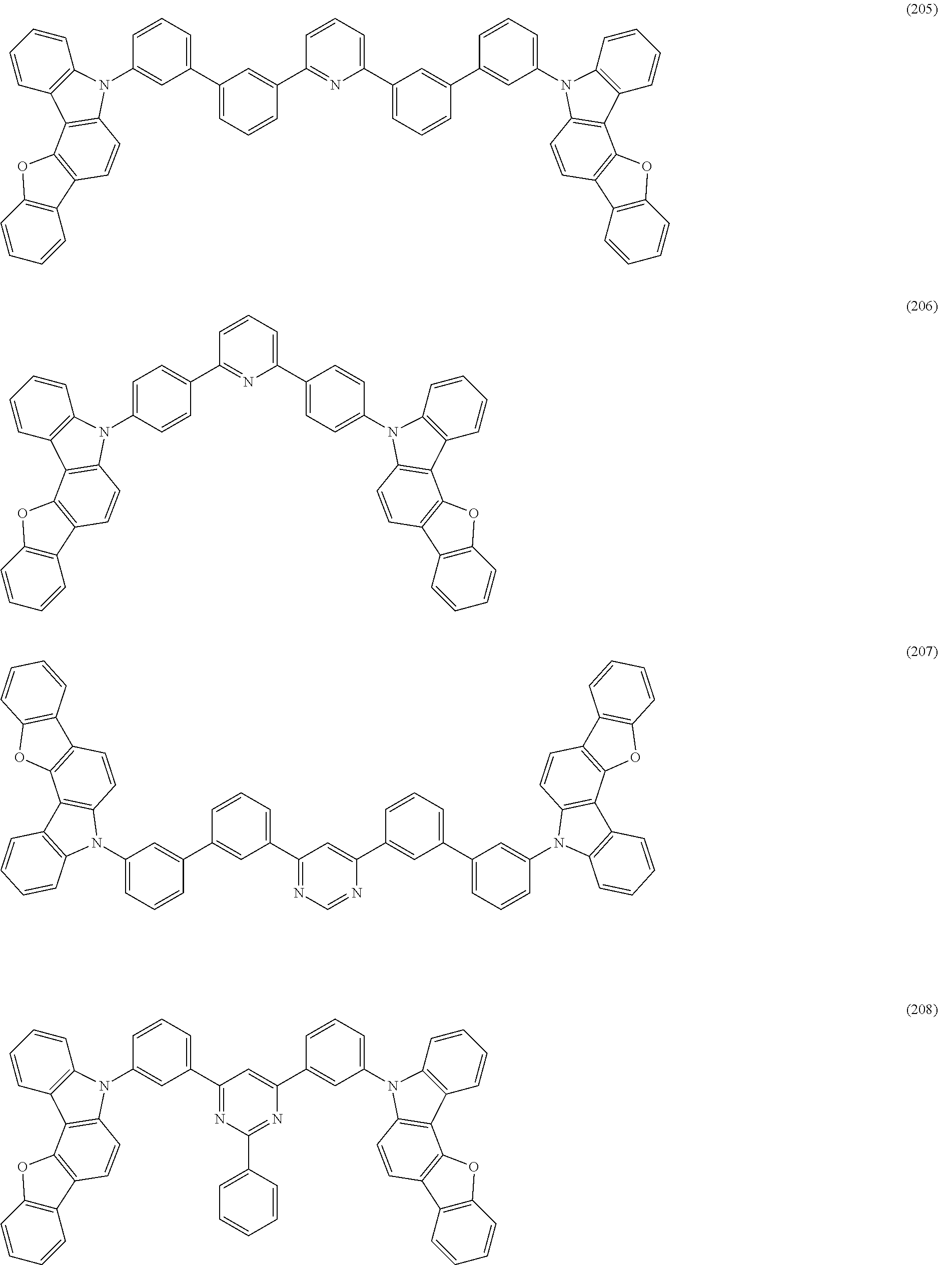

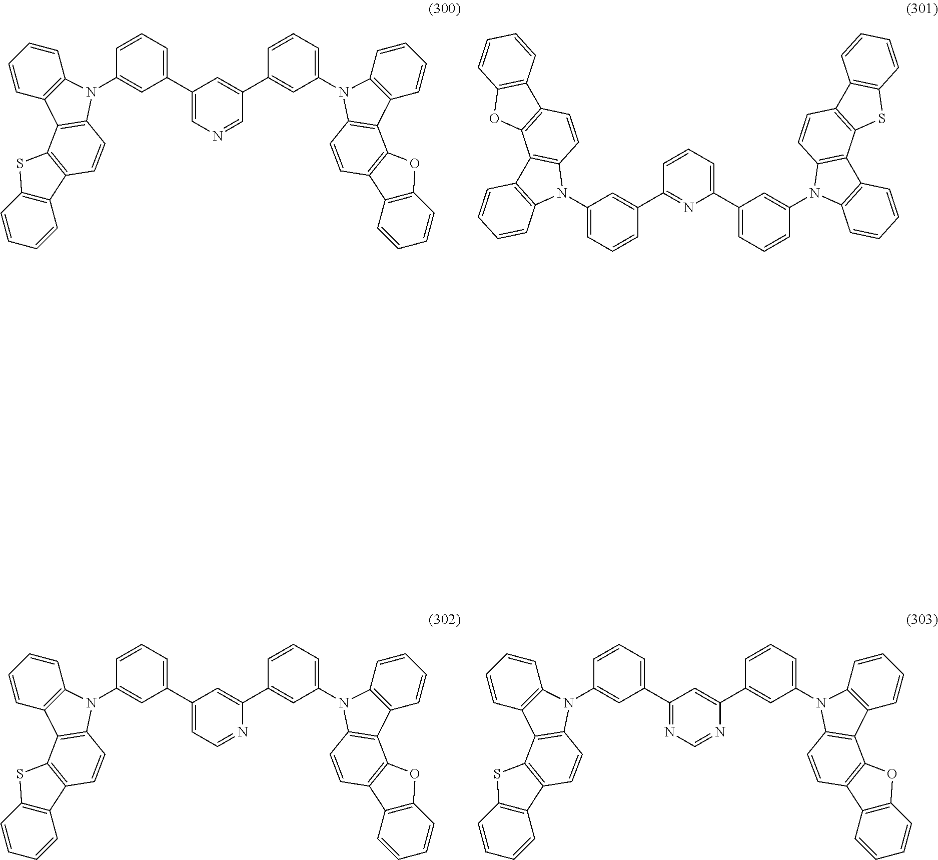

Since the organic compound of one embodiment of the present invention includes a π-electron deficient divalent heteroaromatic group and a plurality of sets each including an arylene group and a heterocyclic skeleton connected to the heteroaromatic group via the arylene group, the thermophysical properties are high, so that a light-emitting element can be more reliable. Furthermore, the electron-transport property of a light-emitting layer can be increased, so that drive voltage can be reduced.

The structures described in this embodiment can be used in appropriate combination with any structure in the other embodiments.

### Embodiment 2

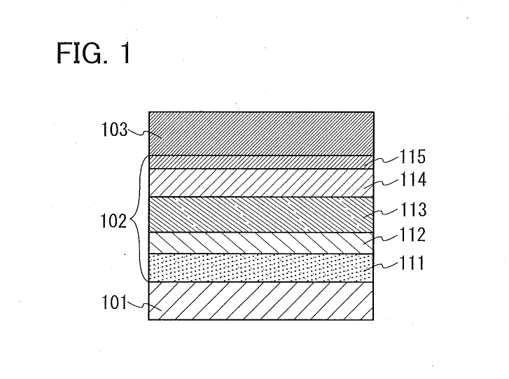

In this embodiment, a light-emitting element in which the organic compound of one embodiment of the present invention can be used as an EL material is described with reference to FIG. 1.

In a light-emitting element described in this embodiment, as illustrated in FIG. 1, an EL layer 102 including a light-emitting layer 113 is interposed between a pair of electrodes (a first electrode (anode) 101 and a second electrode (cathode) 103), and the EL layer 102 includes a hole-injection layer 111, a hole-transport layer 112, an electron-transport layer 114, an electron-injection layer 115, and the like in addition to the light-emitting layer 113.

When voltage is applied to such a light-emitting element, holes injected from the first electrode 101 side and electrons injected from the second electrode 103 side recombine in the light-emitting layer 113 to raise a light-emitting substance contained in the light-emitting layer 113 to an excited state. The light-emitting substance in the excited state emits light when it returns to the ground state.

Although the organic compound of one embodiment of the present invention can be used for any one or more layers in the EL layer 102 described in this embodiment, the heterocyclic compound is preferably used for the light-emitting layer 113, the hole-transport layer 112, or the electron-transport layer 114. In other words, the heterocyclic compound is used in part of a light-emitting element having a structure described below.

A preferred specific example in which the light-emitting element described in this embodiment is fabricated is described below.

As the first electrode (anode) 101 and the second electrode (cathode) 103, a metal, an alloy, an electrically conductive compound, a mixture thereof, and the like can be used. Specific examples are indium oxide-tin oxide (indium tin oxide (ITO)), indium oxide-tin oxide containing silicon or silicon oxide, indium oxide-zinc oxide (indium zinc oxide), indium oxide containing tungsten oxide and zinc oxide, gold (Au), platinum (Pt), nickel (Ni), tungsten (W), chromium (Cr), molybdenum (Mo), iron (Fe), cobalt (Co), copper (Cu), palladium (Pd), titanium (Ti), silver (Ag), and aluminum (Al). In addition, an element belonging to Group 1 or Group 2 of the periodic table, for example, an alkali metal such as lithium (Li) or cesium (Cs), an alkaline earth metal such as calcium (Ca) or strontium (Sr), magnesium (Mg), an alloy containing such an element (MgAg or AILi), a rare earth metal such as europium (Eu) or ytterbium (Yb), an alloy containing such an element, graphene, and the like can be used. The first electrode (anode) 101 and the second electrode (cathode) 103 can be formed by, for example, a sputtering method or an evaporation method (including a vacuum evaporation method).

The hole-injection layer 111 injects holes into the light-emitting layer 113 through the hole-transport layer 112 having a high hole-transport property. The hole-injection layer 111 contains a substance having a high hole-transport property and an acceptor substance, so that electrons are extracted from the substance having a high hole-transport property by the acceptor substance to generate holes and the holes are injected into the light-emitting layer 113 through the hole-transport layer 112. The hole-transport layer 112 is formed using a substance having a high hole-transport property.

Specific examples of the substance having a high hole-transport property, which is used for the hole-injection layer 111 and the hole-transport layer 112, include aromatic amine compounds such as 4,4′-bis[N-(1-naphthyl)-N-phenylamino]biphenyl (abbreviation: NPB or a-NPD), N,N′-bis(3-methylphenyl)-N,N′-diphenyl-[1,1′biphenyl]-4,4′-diamine (abbreviation: TPD), 4,4′,4″-tris(carbazol-9-yl)triphenylamine (abbreviation: TCTA), 4,4′,4″-tris(N,N-diphenylamino)triphenylamine (abbreviation: TDATA), 4,4′,4″-tris [N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: B SPB); 3-[N-(9-phenylcarbazol-3-yl)-N-phenylamino]-9-phenylcarbazole (abbreviation: PCzPCA1); 3,6-bis[N-(9-phenylcarbazol-3-yl)-N-phenylamino]-9-phenylcarbazole (abbreviation: PCzPCA2); and 3-[N-(1-naphthyl)-N-(9-phenylcarbazol-3-yl)amino]-9-phenylcarbazole (abbreviation: PCzPCN1). Other examples include carbazole derivatives such as 4,4′-di(N-carbazolyObiphenyl (abbreviation: CBP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA). The substances listed here are mainly ones−that have a hole mobility of 10⁻⁶ cm²/Vs or higher. Note that any substance other than the substances listed here may be used as long as the hole-transport property is higher than the electron-transport property.

A high molecular compound such as poly(N-vinylcarbazole) (abbreviation: PVK), poly(-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), or poly[N,N′-bis(4-butylphenyl)-N,N′-bis(phenyl)benzidine] (abbD) can also be used.

Examples of the acceptor substance that is used for the hole-injection layer 111 include oxides of metals belonging to Groups 4 to 8 of the periodic table. Specifically, molybdenum oxide is particularly preferable.

The light-emitting layer 113 is a layer containing a light-emitting substance. The light-emitting layer 113 may contain only a light-emitting substance; alternatively, an emission center substance (guest material) may be dispersed in a host material in the light-emitting layer 113. Note that as the host material, the above-described substance having a high hole-transport property or a later-described substance having a high electron-transport property can be used, and preferably, a substance having high triplet excitation energy is used. In addition, the organic compound described in Embodiment 1, which is one embodiment of the present invention, can be used in combination.

There is no particular limitation on the materials that can be used as the light-emitting substance and the emission center substance in the light-emitting layer 113. A light-emitting substance converting singlet excitation energy into luminescence (hereinafter, referred to as fluorescent substance) or a light-emitting substance converting triplet excitation energy into luminescence (hereinafter, referred to as phosphorescent substance) can be used. Examples of the light-emitting substance and the emission center substance are given below.

As an example of the light-emitting substance converting singlet excitation energy into luminescence, a substance emitting fluorescence can be given.

Examples of the substance emitting fluorescence include N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra(tert-butyl)perylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC 1), coumarin 30, N-(9,10-diphenyl-2-anthryl)-N,9-diphenyl-9H-carbazol-3-amine (abbreviation: 2PCAPA),

N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), {2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), {2-tert-butyl-6-[2-(1,1,7,7-tetramethyl-2,3,6,7-tetrahydro-1H,5H-benzo[ij]quinolizin-9-ypethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: B isDCM), and 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM).

Examples of the light-emitting substance converting triplet excitation energy into luminescence include a substance emitting phosphorescence and a thermally activated delayed fluorescence (TADF) material. Note that “delayed fluorescence” exhibited by the TADF material refers to light emission having the same spectrum as normal fluorescence and an extremely long lifetime. The lifetime is le seconds or longer, preferably 10⁻³ seconds or longer.

Examples of the substance emitting phosphorescence include bis{2-[3′,5′-bis(trifluoromethyl)phenyl]pyridinato-N,C^{2′}}iridium(III) picolinate (abbreviation: Ir(CF₃ppy)₂(pic)), bis[2-(4′,6′-difluorophenyl)pyridinato-N,C^{2′}]iridium(III) acetylacetonate (abbreviation: FIracac), tris(2-phenylpyridinato)iridium(III) (abbreviation: Ir(ppy)₃), bis(2-phenylpyridinato)iridium(III) acetylacetonate (abbreviation: Ir(ppy)₂(acac)), tris(acetylacetonato) (monophenanthroline)terbium(III) (abbreviation: Tb(acac)₃(Phen)), bis(benzo[h]quinolinato)iridium(III) acetylacetonate (abbreviation: Ir(bzq)₂(acac)), bis(2,4-diphenyl-1,3-oxazolato-N,C^{2′})iridium(III) acetylacetonate (abbreviation: Ir(dpo)₂(acac)), bis{2-[4′-(perfluorophenyl)phenyl]pyridinato-N,C^{2′}} iridium(III) acetylacetonate (abbreviation: Ir(p-PF-ph)₂(acac)), bis(2-phenylbenzothiazolato-N,C^{2′})iridium(III) acetylacetonate (abbreviation: Ir(bt)₂(acac)), bis[2-(2′-benzo[4,5-a]thienylpyridinato-N,C^{3′}]iridium(III) acetylacetonate (abbreviation: Ir(btp)₂(acac)), bis(1-phenylisoquinolinato-N,C^{2′})iridium (III) acetylacetonate (abbreviation: Ir(piq)₂(acac)), (acetylacetonato)bis[2,3-bis(4-fluorophenyl)quinoxalinato]iridium(III) (abbreviation: Ir(Fdpq)₂(acac)), (acetylacetonato)bis(3,5-dimethyl-2-phenylpyrazinato)iridium(III) (abbreviation: [Ir(mppr-Me)₂(acac)]), (acetylacetonato)bis(5-isopropyl-3-methyl-2-phenylpyrazinato)iridium(III) (abbreviation: [Ir(mppr-iPr)₂(acac)]), (acetylacetonato)bis(2,3,5-triphenylpyrazinato)iridium(III) (abbreviation: Ir(tppr)₂(acac)), bis(2,3,5-triphenylpyrazinato) (dipivaloylmethanato)iridium(III) (abbreviation: [Ir(tppr)₂(dpm)], (acetylacetonato)bis(6-tert-butyl-4-phenylpyrimidinato)iridium(III) (abbreviation: [Ir(tBuppm)₂(acac)]), (acetylacetonato)bis(4,6-diphenylpyrimidinato)iridium(III) (abbreviation: [Ir(dppm)₂(acac)]), 2,3,7,8,12,13,17,18-octaethyl-21H,23H-porphyrin platinum(II) (abbreviation: PtOEP), tris(1,3-diphenyl-1,3-propanedionato) (monophenanthroline)europium(III) (abbreviation: Eu(DBM)₃(Phen)), and tris [1-(2-thenyl)-3 ,3 ,3-trifluoroacetonato] (monophenanthroline)europium(III) (abbreviation: Eu(TTA)₃(Phen)).

Preferable examples of the substance (i.e., host material) used for dispersing the light-emitting substance converting triplet excitation energy into luminescence include compounds having an arylamine skeleton, such as 2,3-bis(4-diphenylaminophenyl)quinoxaline (abbreviation: TPAQn) and NPB, carbazole derivatives such as CBP and 4,4′,4″-tris(carbazol-9-yl)triphenylamine (abbreviation: TCTA), and metal complexes such as bis[2-(2-hydroxyphenyl)pyridinato]zinc (abbreviation: Znpp₂), bis[2-(2-hydroxyphenyl)benzoxazolato]zinc (abbreviation: Zn(BOX)₂), bis(2-methyl-8-quinolinolato)(4-phenylphenolato)aluminum (abbreviation: BAlq), and tris(8-quinolinolato)aluminum (abbreviation: Alq₃). Alternatively, a high molecular compound such as PVK can be used.

Examples of the TADF material includes fullerene, a derivative thereof, an acridine derivative such as proflavine, and eosin. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP). Alternatively, an organic compound including a π-electron rich heteroaromatic ring and a π-electron deficient heteroaromatic ring can be used, such as 2-(biphenyl-4-yl)-4,6-bis(12-phenylindolo [2,3-a]carbazol-11-yl)-1,3 ,5-triazine (PIC-TRZ). Note that a material in which the π-electron rich heteroaromatic ring is directly bonded to the π-electron deficient heteroaromatic ring is particularly preferably used because both the donor property of the π-electron rich heteroaromatic ring and the acceptor property of the π-electron deficient heteroaromatic ring are increased and the energy difference between the S₁ level and the T₁ level becomes small.

When the light-emitting layer 113 includes one or more kinds of host materials and a light-emitting substance converting singlet excitation energy into luminescence or any of the light-emitting substances converting triplet excitation energy into luminescence (i.e., a guest material), light emission with high emission efficiency can be obtained from the light-emitting layer 113. When two or more kinds of host materials are used, they are preferably a combination that can form an exciplex.

The light-emitting layer 113 may have a stacked structure. In that case, each layer in the stacked structure emits light. For example, fluorescence is obtained from a first light-emitting layer in the stacked structure, and phosphorescence is obtained from a second light-emitting layer stacked over the first layer. Note that the stacking order may be reversed. It is preferable that light emission due to energy transfer from an excited complex to a dopant be obtained from the layer that emits phosphorescence. In the case where blue light emission is obtained from one of the first and second layers, orange or yellow light emission can be obtained from the other layer. Each layer may contain various kinds of dopants.

The electron-transport layer 114 is a layer containing a substance having a high electron-transport property. For the electron-transport layer 114, a metal complex such as tris(8-quinolinolato)aluminum (abbreviation: Alq₃), tris(4-methyl-8-quinolinolato)aluminum (abbreviation: Almq₃), bis(10-hydroxybenzo[h]quinolinato)beryllium (abbreviation: BeBq₂), bis(2-methyl-8-quinolinolato)(4-phenylphenolato)aluminum (abbreviation: BAlq), bis[2-(2-hydroxyphenyl)benzoxazolato]zinc (abbreviation: Zn(BOX)₂), or bis[2-(2-hydroxyphenyl)benzothiazolato]zinc (abbreviation: Zn(BTZ)₂) can be used. Alternatively, a heteroaromatic compound such as 2-(4-biphenylyl)-5-(4-tert-butylphenyl)-1,3,4-oxadiazole (abbreviation: PBD), 1,3-bis[5-(p-tent-butylphenyl)-1,3,4-oxadiazol-2-yl]benzene (abbreviation: OXD-7), 3-(4′-tert-butylphenyl)-4-phenyl-5-(4″-biphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: Bphen), bathocuproine (abbreviation: BCP), or 4,4′-bis(5-methylbenzoxazol-2-yl)stilbene (abbreviation: BzOs) can also be used. A high molecular compound such as poly(2,5-pyridinediyl) (abbreviation: PPy), poly[(9,9-dihexylfluorene-2,7-diyl)-co-(pyridine-3,5-diyl)] (abbreviation: PF-Py) or poly[(9,9-dioctylfluorene-2,7-diyl)-co-(2,2′-bipyridine-6,6′-diyl)] (abbreviation: PF-BPy) can also be used. The substances listed here are mainly ones that have an electron mobility of 1×10⁻⁶ cm²IVs or higher. Note that any substance other than the substances listed here may be used for the electron-transport layer 114 as long as the electron-transport property is higher than the hole-transport property. The organic compound described in Embodiment 1, which is one embodiment of the present invention, can also be used.

The electron-transport layer 114 is not limited to a single layer, but may be a stack of two or more layers each containing any of the above substances.

The electron-injection layer 115 is a layer containing a substance having a high electron-injection property. For the electron-injection layer 115, an alkali metal, an alkaline earth metal, or a compound thereof, such as lithium fluoride (LiF), cesium fluoride (CsF), calcium fluoride (CaF₂), or lithium oxide (LiOₓ), can be used. A rare earth metal compound like erbium fluoride (ErF₃) can also be used. An electride may also be used for the electron-injection layer 115. Examples of the electride include a substance in which electrons are added at high concentration to calcium oxide-aluminum oxide. Any of the substances for forming the electron-transport layer 114, which are given above, can be used.

A composite material in which an organic compound and an electron donor (donor) are mixed may also be used for the electron-injection layer 115. Such a composite material is excellent in an electron-injection property and an electron-transport property because electrons are generated in the organic compound by the electron donor. In this case, the organic compound is preferably a material that is excellent in transporting the generated electrons. Specifically, for example, the substances for forming the electron-transport layer 114 (e.g., a metal complex or a heteroaromatic compound), which are given above, can be used. As the electron donor, a substance showing an electron-donating property with respect to the organic compound may be used. Specifically, an alkali metal, an alkaline earth metal, and a rare earth metal are preferable, and lithium, cesium, magnesium, calcium, erbium, and ytterbium are given. In addition, an alkali metal oxide or an alkaline earth metal oxide is preferable, and lithium oxide, calcium oxide, and barium oxide are given. A Lewis base such as magnesium oxide can also be used. An organic compound such as tetrathiafulvalene (abbreviation: TTF) can also be used.

Note that each of the above-described hole-injection layer 111, hole-transport layer 112, light-emitting layer 113, electron-transport layer 114, and electron-injection layer 115 can be formed by a method such as an evaporation method (e.g., a vacuum evaporation method), an ink-jet method, or a coating method.

In the above-described light-emitting element, holes and electrons are recombined in the EL layer 102, whereby light is emitted. Then, the emitted light is extracted outside through one or both of the first electrode 101 and the second electrode 103. Thus, one or both of the first electrode 101 and the second electrode 103 are. electrodes having light-transmitting properties.

The light-emitting element described in this embodiment is an example of a light-emitting element in which the organic compound of one embodiment of the present invention is used as an EL material. The use of the organic compound of one embodiment of the present invention allows high emission efficiency of a light-emitting element that emits phosphorescence with a wavelength shorter than that of green because of the high T₁ level of the organic compound. Further, the use of the organic compound of one embodiment of the present invention allows high reliability of a light-emitting element because the good thennophysical properties of the organic compound render the film quality difficult to change even after driving in a high-temperature enviromnent and accordingly characteristics of the element is difficult to change.

Note that the structure described in this embodiment can be combined as appropriate with any of the structures described in the other embodiments.

### Embodiment 3

Described in this embodiment is a light-emitting element (hereinafter, a tandem light-emitting element) which has a structure in which a charge-generation layer is provided between a plurality of EL layers and the organic compound of one embodiment of the present invention is used as an EL material in the EL layers.

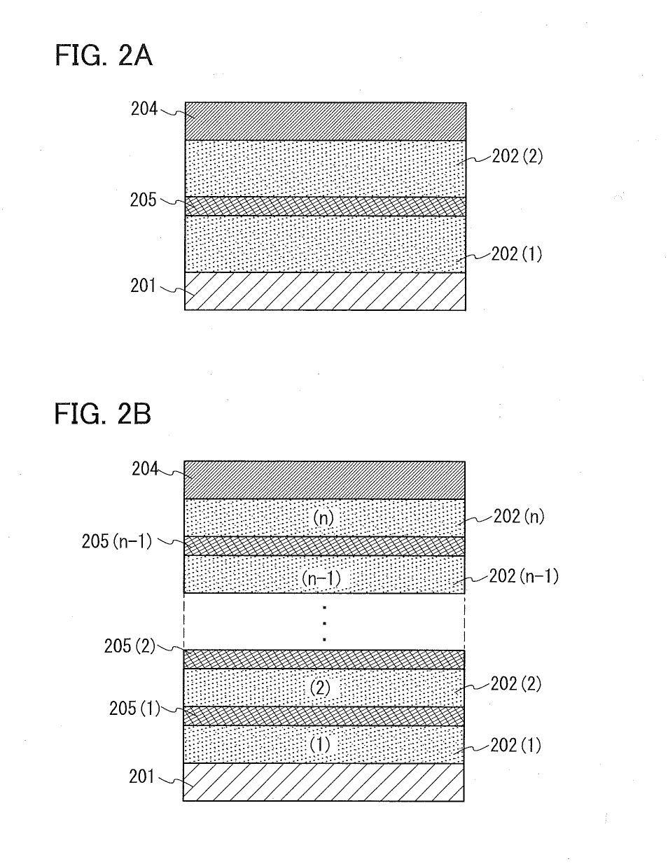

A light-emitting element described in this embodiment is a tandem light-emitting element including a plurality of EL layers (a first EL layer 202(1) and a second EL layer 202(2)) between a pair of electrodes (a first electrode 201 and a second electrode 204), as illustrated in FIG. 2A.

In this embodiment, the first electrode 201 functions as an anode, and the second electrode 204 functions as a cathode. Note that the first electrode 201 and the second electrode 204 can have structures similar to those described in Embodiment 2. In addition, either or both of the EL layers (the first EL layer 202(1) and the second EL layer 202(2)) may have structures similar to those described in Embodiment 2. In other words, the structures of the first EL layer 202(1) and the second EL layer 202(2) may be the same or different from each other and can be similar to those of the EL layers described in Embodiment 2.

In addition, a charge-generation layer 205 is provided between the plurality of EL layers (the first EL layer 202(1) and the second EL layer 202(2)). The charge-generation layer 205 has a function of injecting electrons into one of the EL layers and injecting holes into the other of the EL layers when voltage is applied between the first electrode 201 and the second electrode 204. In this embodiment,. when voltage is applied such that the potential of the first electrode 201 is higher than that of the second electrode 204, the charge-generation layer 205 injects electrons into the first EL layer 202(1) and injects holes into the second EL layer 202(2).

Note that in terms of light extraction efficiency, the charge-generation layer 205 preferably has a property of transmitting visible light (specifically, the charge-generation layer (I) 205 has a visible light transmittance of 40% or more). The charge-generation layer 205 functions even when it has lower conductivity than the first electrode 201 or the second electrode 204.

The charge-generation layer 205 may have either a structure in which an electron acceptor (acceptor) is added to an organic compound having a high hole-transport property or a structure in which an electron donor (donor) is added to an organic compound having a high electron-transport property. Alternatively, both of these structures may be stacked.

In the case of the structure in which an electron acceptor is added to an organic compound having a high hole-transport property, as the organic compound having a high hole-transport property, for example, an aromatic amine compound such as NPB, TPD, TDATA, MTDATA, or BSPB, or the like can be used. The substances listed here are mainly ones that have a hole mobility of 10⁻⁶ cm²/Vs or higher. Note that any organic compound other than the compounds listed here may be used as long as the hole-transport property is higher than the electron-transport property.

As the electron acceptor, 7,7,8,8-tetracyano-2,3,5,6-tetrafluoroquinodimethane (abbreviation: F₄-TCNQ), chloranil, and the like can be given. Oxides of metals belonging to Groups 4 to 8 of the periodic table can also be given. Specifically, vanadium oxide, niobium oxide, tantalum oxide, chromium oxide, molybdenum oxide, tungsten oxide, manganese oxide, and rhenium oxide are preferable because of their high electron-accepting properties. Among these, molybdenum oxide is especially preferable because it is stable in the air, has a low hygroscopic property, and is easy to handle.

In the case of the structure in which an electron donor is added to an organic compound having a high electron-transport property, as the organic compound having a high electron-transport property, for example, a metal complex having a quinoline skeleton or a benzoquinoline skeleton, such as Alq, Almq₃, BeBq₂, or BAlq, or the like can be used. Alternatively, a metal complex having an oxazole-based ligand or a thiazole-based ligand, such as Zn(BOX)₂ or Zn(BTZ)₂ can be used. Alternatively, in addition to such a metal complex, PBD, OXD-7, TAZ, Bphen, BCP, or the like can be used. The substances listed here are mainly ones that have an electron mobility of 10⁻⁶ cm²/Vs or higher. Note that any organic compound other than the compounds listed here may be used as long as the electron-transport property is higher than the hole-transport property.

As the electron donor, it is possible to use an alkali metal, an alkaline earth metal, a rare earth metal, metals belonging to Groups 2 and 13 of the periodic table, or an oxide or carbonate thereof. Specifically, lithium (Li), cesium (Cs), magnesium (Mg), calcium (Ca), ytterbium (Yb), indium (In), lithium oxide, cesium carbonate, or the like is preferably used. Alternatively, an organic compound such as tetrathianaphthacene may be used as the electron donor.

Note that forming the charge-generation layer 205 by using any of the above materials can suppress a drive voltage increase caused by the stack of the EL layers.

Although the light-emitting element including two EL layers is described in this embodiment, the present invention can be similarly applied to a light-emitting element in which n EL layers (202(1) to 202(n)) (n is three or more) are stacked as illustrated in FIG. 2B. In the case where a plurality of EL layers are included between a pair of electrodes as in the light-emitting element according to this embodiment, by providing charge-generation layers (205(1) to 205(n-1)) between the EL layers, light emission in a high luminance region can be obtained with current density kept low. Since the current density can be kept low, the element can have a long lifetime. When the light-emitting element is applied to light-emitting devices, electronic devices, and lighting devices each having a large light-emitting area, voltage drop due to resistance of an electrode material can be reduced, which results in uniform light emission in a large area.

When the EL layers have different emission colors, a desired emission color can be obtained from the whole light-emitting element. For example, in the light-emitting element having two EL layers, when an emission color of the first EL layer and an emission color of the second EL layer are made to be complementary colors, the light-emitting element can emit white light as a whole. Note that “complementary colors” refer to colors that can produce an achromatic color when mixed. In other words, emission of white light can be obtained by mixture of light emitted from substances whose emission colors are complementary colors. Specifically, a combination in which blue light emission is obtained from the first EL layer and yellow light emission or orange light emission is obtained from the second EL layer is given as an example. In that case, it is not necessary that both of blue light emission and yellow (or orange) light emission are fluorescence, and the both are not necessarily phosphorescence. For example, a combination in which blue light emission is fluorescence and yellow (or orange) light emission is phosphorescence or a combination in which blue light emission is phosphorescence and yellow (or orange) light emission is fluorescence may be employed.

The same can be applied to a light-emitting element having three EL layers. For example, the light-emitting element as a whole can provide white light emission when the emission color of the first EL layer is red, the emission color of the second EL layer is green, and the emission color of the third EL layer is blue.

Note that the structure described in this embodiment can be combined as appropriate with any of the structures described in the other embodiments.

### Embodiment 4

Described in this embodiment is a light-emitting device that includes a light-emitting element in which the organic compound of one embodiment of the present invention is used for an EL layer.

The light-emitting device may be either a passive matrix type light-emitting device or an active matrix type light-emitting device. Note that any of the light-emitting elements described in the other embodiments can be used for the light-emitting device described in this embodiment.

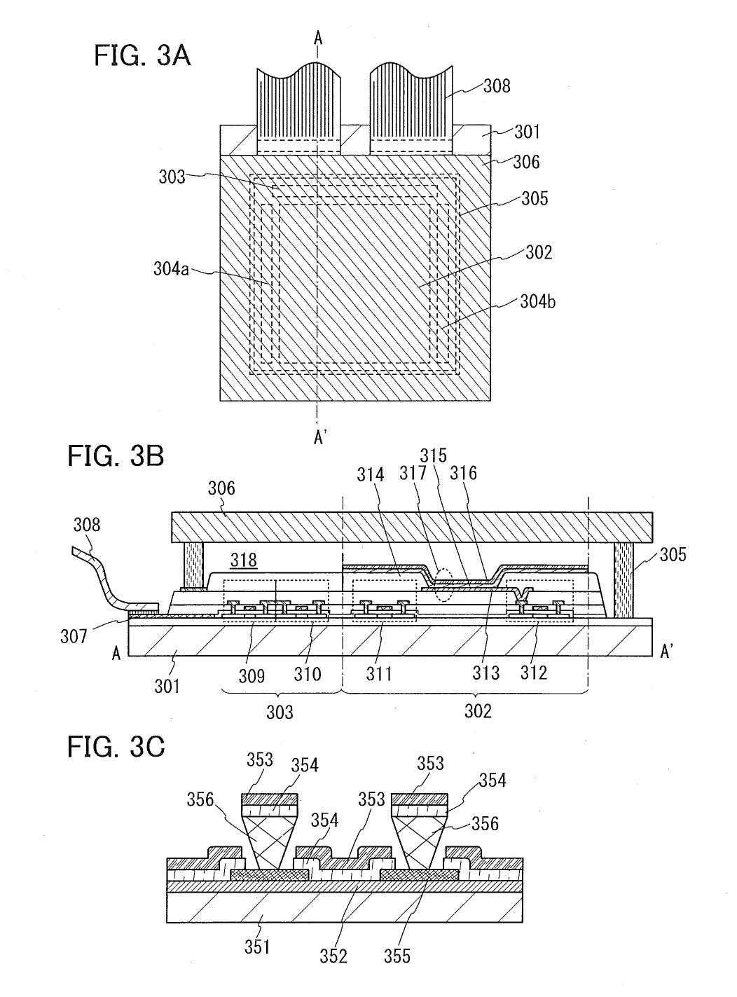

In this embodiment, an active matrix light-emitting device is described with reference to FIGS. 3A and 3B.

Note that FIG. 3A is a top view illustrating a light-emitting device and FIG. 3B is a cross-sectional view taken along the chain line A-A′ in FIG. 3A. The active matrix light-emitting device according to this embodiment includes a pixel portion 302 provided over an element substrate 301, a driver circuit portion (a source line driver circuit) 303, and driver circuit portions (gate line driver circuits) 304a and 304b. The pixel portion 302, the driver circuit portion 303, and the driver circuit portions 304a and 304b are sealed between the element substrate 301 and a sealing substrate 306 with a sealant 305.

In addition, over the element substrate 301, a lead wiring 307 for connecting an external input terminal, through which a signal (e.g., a video signal, a clock signal, a start signal, a reset signal, or the like) or electric potential from the outside is transmitted to the driver circuit portion 303 and the driver circuit portions 304a and 304b, is provided. Here, an example is described in which a flexible printed circuit (FPC) 308 is provided as the external input terminal. Although only the FPC is illustrated here, the FPC may be provided with a printed wiring board (PWB). The light-emitting device in this specification includes, in its category, not only the light-emitting device itself but also the light-emitting device provided with the FPC or the PWB.

Next, a cross-sectional structure is described with reference to FIG. 3B. The driver circuit portion and the pixel portion are formed over the element substrate 301; the driver circuit portion 303, which is the source line driver circuit, and the pixel portion 302 are illustrated here.

The driver circuit portion 303 is an example in which an FET 309 and an FET 310 are combined. Note that the driver circuit portion 303 may be formed with a circuit including transistors having the same conductivity type (either n-channel transistors or p-channel transistors) or a CMOS circuit including an n-channel transistor and a p-channel transistor. Although this embodiment shows a driver integrated type in which the driver circuit is formed over the substrate, the driver circuit is not necessarily formed over the substrate, and may be formed outside the substrate.

The pixel portion 302 includes a plurality of pixels each of which includes a switching FET 311, a current control FET 312, and a first electrode (anode) 313 which is electrically connected to a wiring (a source electrode or a drain electrode) of the current control FET 312. Although the pixel portion 302 includes two FETs, the switching FET 311 and the current control FET 312, in this embodiment, one embodiment of the present invention is not limited thereto. The pixel portion 302 may include, for example, three or more FETs and a capacitor in combination.

As the FETs 309, 310, 311, and 312, for example, a staggered transistor or an inverted staggered transistor can be used. For example, a Group 13 semiconductor (e.g., gallium), a Group 14 semiconductor (e.g., silicon), a compound semiconductor, an oxide semiconductor, or an organic semiconductor can be used. In addition, there is no particular limitation on the crystallinity of the semiconductor material, and an amorphous semiconductor or a crystalline semiconductor can be used. In particular, an oxide semiconductor is preferably used for the FETs 309, 310, 311, and 312. Examples of the oxide semiconductor include an In-Ga oxide and an In-M-Zn oxide (M is Al, Ga, Y, Zr, La, Ce, or Nd). For example, an oxide semiconductor that has an energy gap of 2 eV or more, preferably 2.5 eV or more, further preferably 3 eV or more is used for the FETs 309, 310, 311, and 312, so that the off-state current of the transistors can be reduced.

In addition, an insulator 314 is formed to cover end portions of the first electrode (anode) 313. In this embodiment, the insulator 314 is formed using a positive photosensitive acrylic resin. The first electrode 313 is used as an anode in this embodiment.

The insulator 314 preferably has a curved surface with curvature at an upper end portion or a lower end portion thereof. This enables the coverage with a film to be formed over the insulator 314 to be favorable. The insulator 314 can be formed using, for example, either a negative photosensitive resin or a positive photosensitive resin. The material of the insulator 314 is not limited to an organic compound and an inorganic compound such as silicon oxide, silicon oxynitride, or silicon nitride can also be used.

An EL layer 315 and a second electrode (cathode) 316 are stacked over the first electrode (anode) 313. In the EL layer 315, at least a light-emitting layer is provided. In the EL layer 315, a hole-injection layer, a hole-transport layer, an electron-transport layer, an electron-injection layer, a charge-generation layer, and the like can be provided as appropriate in addition to the light-emitting layer.

A light-emitting element 317 is formed of a stack of the first electrode (anode) 313, the EL layer 315, and the second electrode (cathode) 316. For the first electrode (anode) 313, the EL layer 315, and the second electrode (cathode) 316, any of the materials given in Embodiment 2 can be used. Although not illustrated, the second electrode (cathode) 316 is electrically connected to the FPC 308, which is an external input terminal.

Although the cross-sectional view of FIG. 3B illustrates only one light-emitting element 317, a plurality of light-emitting elements are arranged in a matrix in the pixel portion 302. Light-emitting elements that emit light of three kinds of colors (R, G, and B) are selectively formed in the pixel portion 302, whereby a light-emitting device capable of full color display can be obtained. In addition to the light-emitting elements that emit light of three kinds of colors (R, G, and B), for example, light-emitting elements that emit light of white (W), yellow (Y), magenta (M), cyan (C), and the like may be formed. For example, the light-emitting elements that emit light of a plurality of kinds of colors are used in combination with the light-emitting elements that emit light of three kinds of colors (R, G, and B), whereby effects such as an improvement in color purity and a reduction in power consumption can be obtained. Alternatively, the light-emitting device may be capable of full color display by combination with color filters. The light-emitting device may have improved emission efficiency and reduced power consumption by combination with quantum dots.

Furthermore, the sealing substrate 306 is attached to the element substrate 301 with the sealant 305, whereby a light-emitting element 317 is provided in a space 318 surrounded by the element substrate 301, the sealing substrate 306, and the sealant 305. Note that the space 318 may be filled with an inert gas (such as nitrogen and argon) or the sealant 305. In the case where the sealant is applied and then the substrates are attached, UV treatment, heat treatment, or a combination thereof is preferably performed.

An epoxy-based resin or glass frit is preferably used for the sealant 305. The material preferably allows as little moisture and oxygen as possible to penetrate. As the sealing substrate 306, a glass substrate, a quartz substrate, or a plastic substrate formed of fiber-reinforced plastic (FRP), poly(vinyl fluoride) (PVF), polyester, acrylic, or the like can be used. In the case where glass fit is used as the sealant, the element substrate 301 and the sealing substrate 306 are preferably glass substrates for high adhesion

As described above, an active matrix light-emitting device can be obtained.

As the light-emitting device that includes a light-emitting element in which the organic compound of one embodiment of the present invention is used for an EL layer, a passive matrix light-emitting device can be provided as well as the above active matrix light-emitting device.

FIG. 3C shows a cross-sectional view of a pixel portion in a passive matrix light-emitting device.

A plurality of first electrodes 352 having an island shape as one electrode of the light-emitting element are formed in the form of stripes extending in one direction over a substrate 351. In addition, an insulating film 355 is formed over the first electrodes 352 to cover end portions of the first electrodes 352. This insulating film 355 includes opening portions in part of a region over the first electrodes 352, and EL layers 354 are formed to be in contact with the first electrodes 352 in the opening portions.

Partitions 356 using an insulating material are provided over the insulating film 355. The sidewalls of the partitions 356 slope so that a distance between both the sidewalls is gradually narrowed toward the surface of the substrate. In other words, a cross section taken along the direction of the short side of the partition 356 is trapezoidal, and the lower side (a side which is in the same direction as a plane direction of the insulating film 355 and in contact with the insulating film 355) is shorter than the upper side (a side which is in the same direction as the plane direction of the insulating film 355 and not in contact with the insulating film 355). The partitions 356 thus provided can prevent defects in the light-emitting element due to static electricity or the like.

Over the EL layers 354, second electrodes 353 serving as the other electrode of the light-emitting element are formed. Since the EL layers 354 and the second electrodes 353 are formed after the partitions 356 are formed, the EL layers 354 and the second electrodes 353 are sequentially stacked not only over the first electrodes 352 but also over the partitions 356.

Note that sealing can be performed as in the case of the active matrix light-emitting device and therefore not described here.

As described above, the passive matrix light-emitting device can be obtained.

Note that the structure described in this embodiment can be combined as appropriate with any of the structures described in the other embodiments.

### Embodiment 5

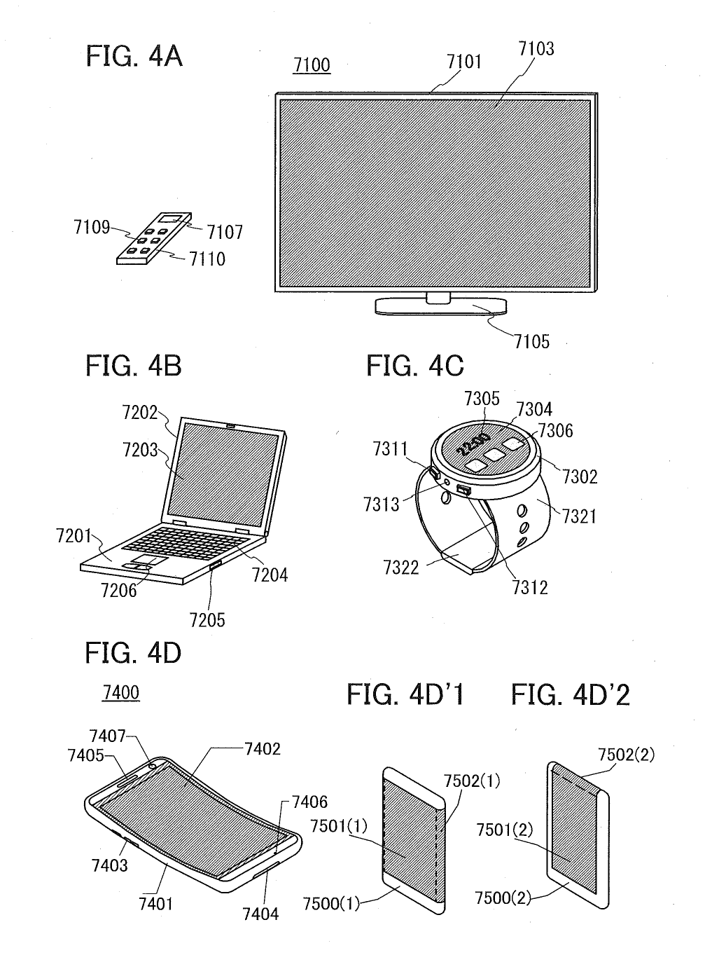

In this embodiment, examples of an electronic device manufactured using a light-emitting device which is one embodiment of the present invention are described with reference to FIGS. 4A to 4D.

Examples of the electronic device including the light-emitting device are television devices (also referred to as TV or television receivers), monitors for computers and the like, cameras such as digital cameras and digital video cameras, digital photo frames, cellular phones (also referred to as portable telephone devices), portable game consoles, portable information temiinals, audio playback devices, large game machines such as pachinko machines, and the like. Specific examples of the electronic devices are illustrated in FIGS. 4A to 4D.

FIG. 4A illustrates an example of a television device. In the television device 7100, a display portion 7103 is incorporated in a housing 7101. The display portion 7103 can display images and may be a touch panel (an input/output device) including a touch sensor (an input device). Note that the light-emitting device of one embodiment of the present invention can be used for the display portion 7103. In addition, here, the housing 7101 is supported by a stand 7105.

The television device 7100 can be operated by an operation switch of the housing 7101 or a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110.

Note that the television device 7100 is provided with a receiver, a modem, and the like. With the use of the receiver, general television broadcasts can be received. Moreover, when the television device is connected to a communication network with or without wires via the modem, one-way (from a sender to a receiver) or two-way (between a sender and a receiver or between receivers) information communication can be performed.

FIG. 4B illustrates a computer, which includes a main body 7201, a housing 7202, a display portion 7203, a keyboard 7204, an external connection port 7205, a pointing device 7206, and the like. Note that this computer can be manufactured using the light-emitting device of one embodiment of the present invention for the display portion 7203. The display portion 7203 may be a touch panel (an input/output device) including a touch sensor (an input device).

FIG. 4C illustrates a smart watch, which includes a housing 7302, a display panel 7304, operation buttons 7311 and 7312, a connection terminal 7313, a band 7321, a clasp 7322, and the like.

The display panel 7304 mounted in the housing 7302 serving as a bezel includes a non-rectangular display region. The display panel 7304 can display an icon 7305 indicating time, another icon 7306, and the like. The display panel 7304 may be a touch panel (an input/output device) including a touch sensor (an input device).

The smart watch illustrated in FIG. 4C can have a variety of functions, for example, a function of displaying a variety of information (e.g., a still image, a moving image, and a text image) on a display portion, a touch panel function, a function of displaying a calendar, date, time, and the like, a function of controlling processing with a variety of software (programs), a wireless communication function, a function of being connected to a variety of computer networks with a wireless communication function, a function of transmitting and receiving a variety of data with a wireless communication function, and a function of reading program or data stored in a recording medium and displaying the program or data on a display portion.

The housing 7302 can include a speaker, a sensor (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone, and the like. Note that the smart watch can be manufactured using the light-emitting device for the display panel 7304.

FIG. 4D illustrates an example of a cellular phone (e.g., smartphone). A cellular phone 7400 includes a housing 7401 provided with a display portion 7402, a microphone 7406, a speaker 7405, a camera 7407, an external connection portion 7404, an operation button 7403, and the like. In the case where a light-emitting device is manufactured by fonning a light-emitting element of one embodiment of the present invention over a flexible substrate, the light-emitting element can be used for the display portion 7402 having a curved surface as illustrated in FIG. 4D.

When the display portion 7402 of the cellular phone 7400 illustrated in FIG. 4D is touched with a finger or the like, data can be input to the cellular phone 7400. In addition, operations such as making a call and composing an e-mail can be performed by touch on the display portion 7402 with a finger or the like.

There are mainly three screen modes of the display portion 7402. The first mode is a display mode mainly for displaying an image. The second mode is an input mode mainly for inputting data such as characters. The third mode is a display-and-input mode in which two modes of the display mode and the input mode are combined.

For example, in the case of making a call or creating e-mail, a character input mode mainly for inputting characters is selected for the display portion 7402 so that characters displayed on the screen can be input. In this case, it is preferable to display a keyboard or number buttons on almost the entire screen of the display portion 7402.

When a detection device such as a gyroscope or an acceleration sensor is provided inside the cellular phone 7400, display on the screen of the display portion 7402 can be automatically changed by determining the orientation of the cellular phone 7400 (whether the cellular phone is placed horizontally or vertically for a landscape mode or a portrait mode).

The screen modes are changed by touch on the display portion 7402 or operation with the operation button 7403 of the housing 7401. The screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, if a signal detected by an optical sensor in the display portion 7402 is detected and the input by touch on the display portion 7402 is not performed for a certain period, the screen mode may be controlled so as to be changed from the input mode to the display mode.

The display portion 7402 may function as an image sensor. For example, an image of a palm print, a fingerprint, or the like is taken by touch on the display portion 7402 with the palm or the finger, whereby personal authentication can be performed. In addition, by providing a backlight or a sensing light source that emits near-infrared light in the display portion, an image of a finger vein, a palm vein, or the like can be taken.

The light-emitting device can be used for a cellular phone having a structure illustrated in FIG. 4D′1 or FIG. 4D′2, which is another structure of the cellular phone (e.g., smartphone).

Note that in the case of the structure illustrated in FIG. 4D′1 or FIG. 4D′2, text data, image data, or the like can be displayed on second screens 7502(1) and 7502(2) of housings 7500(1) and 7500(2) as well as first screens 7501(1) and 7501(2). Such a structure enables a user to easily see text data, image data, or the like displayed on the second screens 7502(1) and 7502(2) while the cellular phone is placed in user's breast pocket.

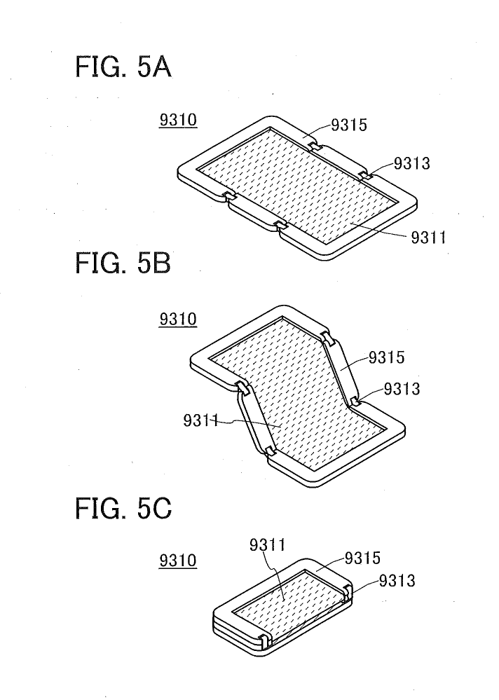

FIGS. 5A to 5C illustrate a foldable portable information terminal 9310. FIG. 5A illustrates the portable information terminal 9310 which is opened. FIG. 5B illustrates the portable information terminal 9310 which is being opened or being folded. FIG. 5C illustrates the portable information terminal 9310 that is folded. The portable information terminal 9310 is highly portable when folded. When the portable information terminal 9310 is opened, a seamless large display region is highly browsable.

A display panel 9311 is supported by three housings 9315 joined together by hinges 9313. Note that the display panel 9311 may be a touch panel (an input/output device) including a touch sensor (an input device). By bending the display panel 9311 at a connection portion between two housings 9315 with the use of the hinges 9313, the portable information terminal 9310 can be reversibly changed in shape from an opened state to a folded state. A light-emitting device of one embodiment of the present invention can be used for the display panel 9311. A display region 9312 is a display region that positioned at a side surface of the portable information terminal 9310 that is folded. On the display region 9312, information icons, file shortcuts of frequently used applications or programs, and the like can be displayed, and confirmation of information and start of application can be smoothly performed.

As described above, the electronic devices can be obtained using the light-emitting device of one embodiment of the present invention. Note that the light-emitting device can be used for electronic devices in a variety of fields without being limited to the electronic devices described in this embodiment.

Note that the structure described in this embodiment can be combined as appropriate with any of the structures described in the other embodiments.

### Embodiment 6

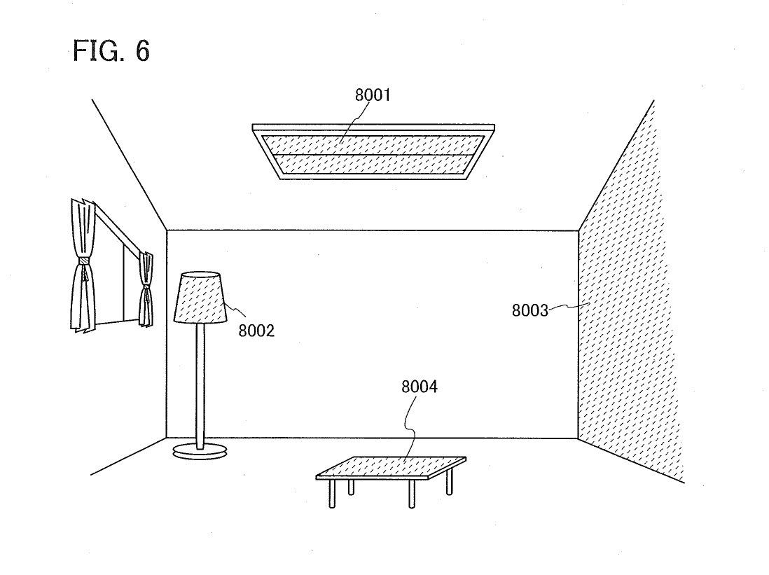

In this embodiment, examples of a lighting device including the light-emitting device of one embodiment of the present invention are described with reference to FIG. 6.

FIG. 6 illustrates an example in which the light-emitting device is used as an indoor lighting device 8001. Since the light-emitting device can have a large area, it can be used for a lighting device having a large area. In addition, with the use of a housing with a curved surface, a lighting device 8002 which includes the housing, a cover, and a support and in which a light-emitting region has a curved surface can also be obtained. A light-emitting element included in the light-emitting device described in this embodiment is in a thin film form, which allows the housing to be designed more freely. Thus, the lighting device can be elaborately designed in a variety of ways. In addition, a wall of the room may be provided with a large-sized lighting device 8003.

When the light-emitting device is used for a table by being used as a surface of a table, a lighting device 8004 that has a function as a table can be obtained. When the light-emitting device is used as part of other furniture, a lighting device that functions as the furniture can be obtained.

As described above, a variety of lighting devices that include the light-emitting device can be obtained. Note that these lighting devices are also embodiments of the present invention.

Note that the structure described in this embodiment can be combined as appropriate with any of the structures described in the other embodiments.

### Embodiment 7

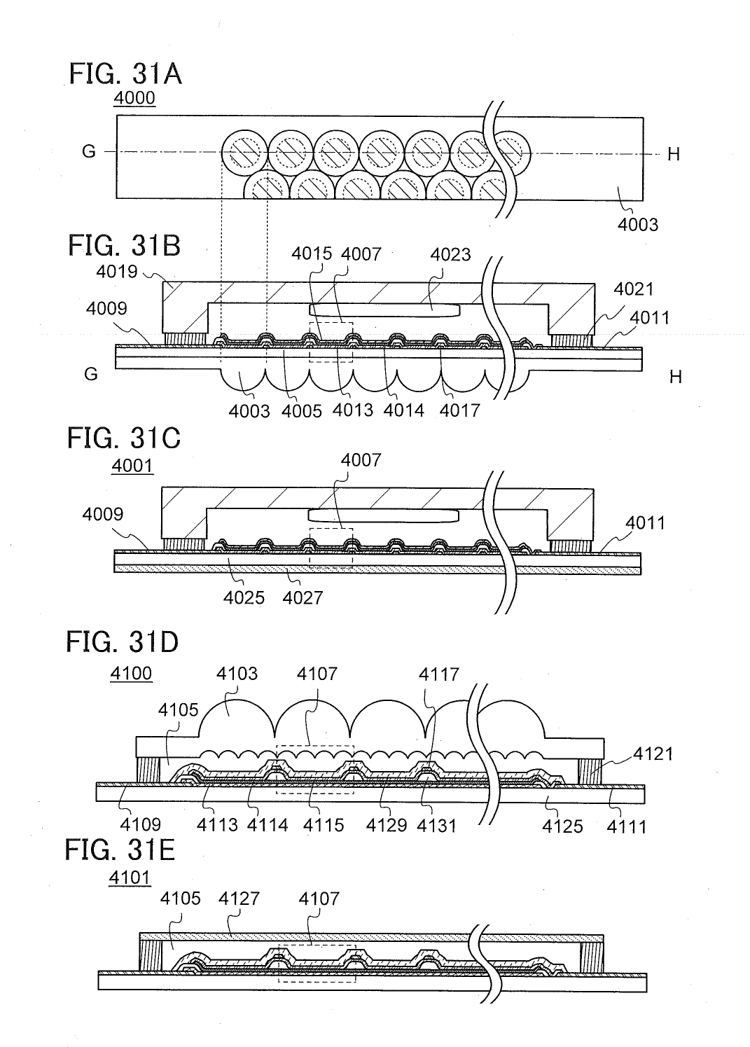

In this embodiment, a structure of a lighting device fabricated using the light-emitting element of one embodiment of the present invention is described with reference to FIGS. 31A to 31E.

FIGS. 31A to 31E are a plan view and cross-sectional views of lighting devices. FIGS. 31A to 31C illustrate bottom-emission lighting devices in which light is extracted from the substrate side. FIG. 31B is a cross-sectional view taken along the dashed-dotted line G-H in FIG. 31A.

A lighting device 4000 illustrated in FIGS. 31A and 31B includes a light-emitting element 4007 over a substrate 4005. In addition, the lighting device includes a substrate 4003 with unevenness on the outside of the substrate 4005. The light-emitting element 4007 includes a lower electrode 4013, an EL layer 4014, and an upper electrode 4015.

The lower electrode 4013 is electrically connected to an electrode 4009, and the upper electrode 4015 is electrically connected to an electrode 4011. In addition, an auxiliary wiring 4017 electrically connected to the lower electrode 4013 may be provided.

The substrate 4005 and a sealing substrate 4019 are bonded to each other by a sealant 4021. A desiccant 4023 is preferably provided between the sealing substrate 4019 and the light-emitting element 4007.

The substrate 4003 has the unevenness illustrated in FIG. 31A, whereby the extraction efficiency of light emitted from the light-emitting element 4007 can be increased. Instead of the substrate 4003, a diffusion plate 4027 may be provided on the outside of the substrate 4025 as in a lighting device 4001 illustrated in FIG. 31C.

FIGS. 31D and 31E illustrate top-emission lighting devices in which light is extracted from the side opposite to the substrate.

A lighting device 4100 illustrated in FIG. 31D includes a light-emitting element 4107 over a substrate 4125. The light-emitting element 4107 includes a lower electrode 4113, an EL layer 4114, and an upper electrode 4115.

The lower electrode 4113 is electrically connected to an electrode 4109, and the upper electrode 4115 is electrically connected to an electrode 4111. An auxiliary wiring 4117 electrically connected to the upper electrode 4115 may be provided. An insulating layer 4131 may be provided under the auxiliary wiring 4117.

The substrate 4125 and a sealing substrate 4103 with unevenness are bonded to each other by a sealant 4121. A planarization film 4105 and a barrier film 4129 may be provided between the sealing substrate 4103 and the light-emitting element 4107.

The sealing substrate 4103 has the unevenness illustrated in FIG. 31D, whereby the extraction efficiency of light emitted from the light-emitting element 4107 can be increased. Instead of the sealing substrate 4103, a diffusion plate 4127 may be provided over the light-emitting element 4107 as in a lighting device 4101 illustrated in FIG. 31E.

The light-emitting element of one embodiment of the present invention can be applied to light-emitting layers included in the EL layer 4014 and the EL layer 4114. The light-emitting element can have low drive voltage, high current efficiency, or a long lifetime; thus, the lighting devices 4000, 4001, 4100, and 4101 can have low power consumption or a long lifetime.

Note that the structure described in this embodiment can be combined with any of the structures described in the other embodiments and the examples as appropriate.

### EXAMPLE 1

### SYNTHESIS EXAMPLE

In this example, a method of synthesizing an organic compound of one embodiment of the present invention, 5,5′-(3,5-pyridinediyldi-3,1-phenylene)bis-5H-benzothieno [3,2-c] carbazole (abbreviation: 3,5mBTcP2Py) (structural formula (100)), is described. Note that a structure of 3,5mBTcP2Py is shown below.

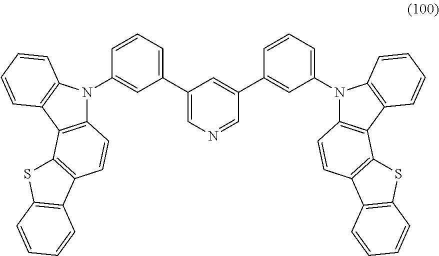 [SMILES: c1cc(-c2cncc(-c3cccc(-n4c5ccccc5c5c6sc7ccccc7c6ccc54)c3)c2)cc(-n2c3ccccc3c3c4sc5ccccc5c4ccc32)c1]

Into a 100-mL three-neck flask were put 0.95 g (4.0 mmol) of 3,5-dibromopyridine, 3.5 g (8.8 mmol) of 3-(5H-benzothieno[3,2-c]carbazol-5-yl)phenyboronic acid, 0.12 g (0.40 mmol) of tris(2-methylphenyl)phosphine, 2.4 g (18 mmol) of potassium carbonate, 8.8 mL of water, 15 mL of toluene, and 5 mL of ethanol. This mixture was degassed by being stirred under reduced pressure, and the air in the flask was replaced with nitrogen.

To the mixture, 18 mg (0.080 mmol) of palladium(II) acetate was added. This mixture was stirred at 90° C. under a nitrogen stream for 8 hours. After that, a precipitated solid was collected by suction filtration. This solid was purified by silica gel column chromatography (using toluene first and then a solvent containing toluene and ethyl acetate in a ratio of 2:1). The obtained fraction was concentrated to give an oily substance. This oily substance was purified by high perfoimance liquid column chromatography (HPLC). The obtained fraction was concentrated to give a solid. This solid was recrystallized from ethyl acetate, and a precipitated solid was collected by suction filtration. Hexane was added to the obtained solid and ultrasonic wave irradiation was performed. A solid was collected by suction filtration to give 2.0 g of a white solid, which was the object of the synthesis, in a yield of 65%.

The synthesis scheme of the above synthesis method is shown below in (A-1).

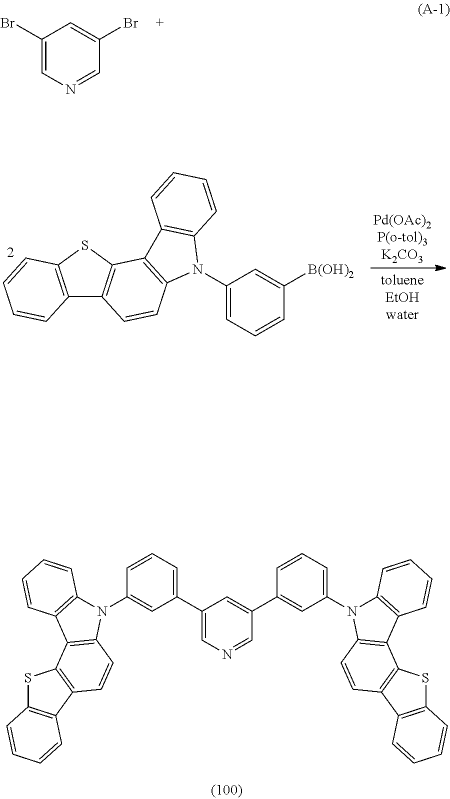

By a train sublimation method, 2.0 g of the obtained white solid was purified. In the purification by sublimation, 3,5mBTcP2Py was heated at 320° C. under the conditions where the pressure was 3.0 Pa and the argon flow rate was 5.0 mL/min. After the purification by sublimation, 0.77 g of a white solid of 3,5mBTcP2Py was obtained at a collection rate of 39%.

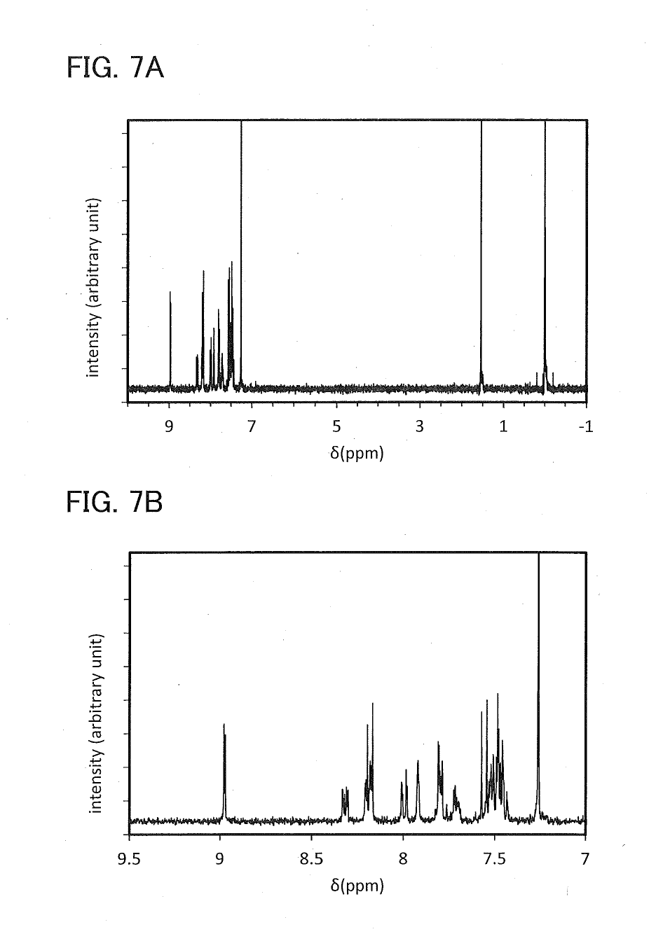

Analysis results by nuclear magnetic resonance (¹H-NMR) spectroscopy of the white solid obtained by the above synthesis method are described below. FIGS. 7A and 7B are ¹H-NMR charts. The results show that the organic compound of one embodiment of the present invention, 3,5mBTcP2Py (structural formula (100)), was obtained.

¹H-NMR (CDCl₃, 300 MHz): δ (ppm)=7.44-7.57 (m, 12H), 7.69-7.72 (m, 2H), 7.79-7.81 (m, 4H), 7.92 (s, 2H), 8.00 (d, J=7.3 Hz, 2H), 8.17-8.21 (m, 5H), 8.31-8.34 (in, 2H), 8.97 (d, J=2.4 Hz, 2H).

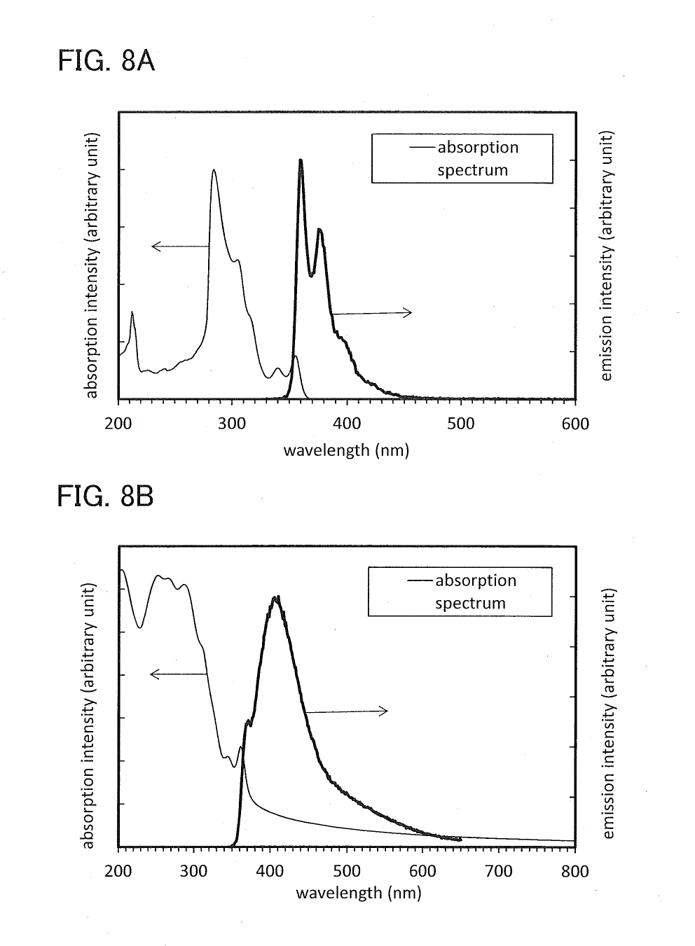

Next, ultraviolet-visible absorption spectra (hereinafter, simply referred to as “absorption spectra”) and emission spectra of 3,SmBTcP2Py in a toluene solution of 3,5mBTcP2Py and a thin film of 3,SmBTcP2Py were measured at room temperature. The spectra of the toluene solution of 3,5mBTcP2Py were measured by putting the solution in a quartz cell. The spectra of the thin film were measured with a sample prepared by evaporation of 3,5mBTcP2Py on a quartz substrate. The absorption spectra were measured with an ultraviolet-visible light spectrophotometer (V-550, manufactured by JASCO Corporation), and the emission spectra were measured with a fluorescence spectrophotometer (FS920, manufactured by Hamamatsu Photonics K.K.). FIG. 8A shows the obtained absorption and emission spectra of 3,5mBTcP2Py in the toluene solution, and FIG. 8B shows the obtained absorption and emission spectra of the thin film. The horizontal axis represents wavelength and the vertical axes represent absorption intensity and emission intensity. In each of FIGS. 8A and 8B, two solid lines are shown; the thin line represents the absorption spectrum, and the thick line represents the emission spectrum. The absorption spectrum shown in FIG. 8A was obtained by subtraction of the absorption spectra of toluene and the quartz cell from the obtained absorption spectrum. The absorption spectrum shown in FIG. 8B was obtained by subtraction of the absorption spectrum of the quartz substrate from the obtained absorption spectrum.

As shown in FIGS. 8A and 8B, 3,5mBTcP2Py, which is one embodiment of the present invention, in the toluene solution has absorption peaks at approximately 355 nm, 340 nm, 305 nm, and 284 nm, and emission peaks at approximately 359 nm and 380 nm. In addition, the thin film of 3,SmBTcP2Py has absorption peaks at approximately 361 nm, 345 nm, 310 nm, and 286 nm, and emission peaks at approximately 371 nm and 405 nm Thus, it was found that absorption and emission of 3,5mBTcP2Py occur in extremely short wavelength regions.

Next, 3,5mBTcP2Py, which is one embodiment of the present invention, was analyzed by liquid chromatography mass spectrometry (LC/MS).

The analysis by LC/MS was carried out with Acquity UPLC (manufactured by Waters Corporation), and Xevo G2 Tof MS (manufactured by Waters Corporation).

In the MS, ionization was carried out by an electrospray ionization (abbreviation: ESI) method. At this time, the capillary voltage and the sample cone voltage were set to 3.0 kV and 30 V, respectively, and detection was performed in a positive mode. A component that underwent the ionization under the above-described conditions was collided with an argon gas in a collision cell to dissociate into product ions. The energy (collision energy) for the collision with argon was 70 eV. The mass range for the measurement was m/z=100 to 1200.

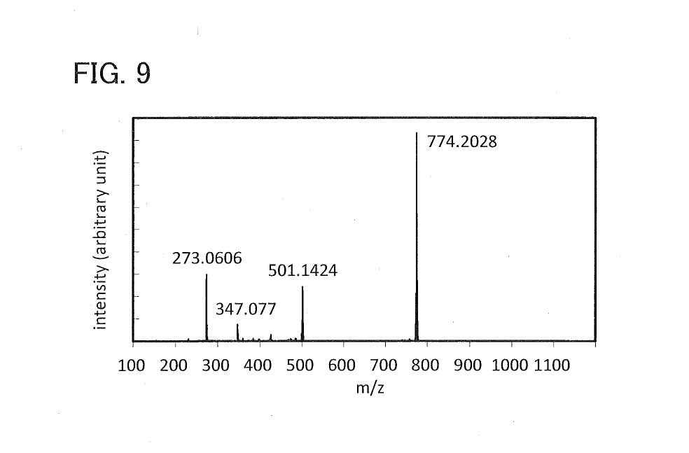

FIG. 9 shows the measurement results. The results in FIG. 9 reveal that the product ions of 3,5mBTcP2Py, which is the organic compound of one embodiment of the present invention represented by the structural formula (100), are detected mainly around m/z=774, around m/z=501, around m/z=273, and around m/z=347. Note that the results in FIG. 9 show characteristics derived from 3,5mBTcP2Py and thus can be regarded as important data for identifying 3,5mBTcP2Py contained in a mixture.

### EXAMPLE 2

### SYNTHESIS EXAMPLE 2

In this example, a method of synthesizing an organic compound of one embodiment of the present invention, 5,5′-(3,5-pyridinediyldi-3,1-phenylene)bis-5H-benzofuro[3,2-c]carbazole (abbreviation: 3,5mBFcP2Py) (structural formula (200)), is described. Note that a structure of 3,5mBFcP2Py is shown below.

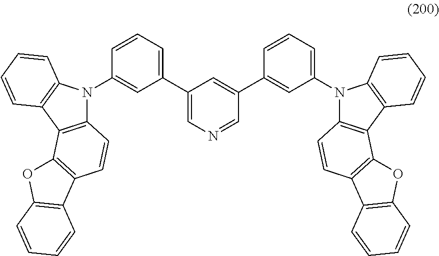 [SMILES: c1cc(-c2cncc(-c3cccc(-n4c5ccccc5c5c6oc7ccccc7c6ccc54)c3)c2)cc(-n2c3ccccc3c3c4oc5ccccc5c4ccc32)c1]

Into a 100-mL three-neck flask were put 1.2 g (4.8 mmol) of 3,5-dibromopyridine, 4.0 g (11 mmol) of 3-(5H-benzofuro[3,2-c]carbazol-5-yl)phenyboronic acid, 0.15 g (0.48 mmol) of tris(2-methylphenyl)phosphine, 2.9 g (21 mmol) of potassium carbonate, 18 mL of toluene, 6.0 mL of ethanol, and 10 mL of water. This mixture was degassed by being stirred under reduced pressure, and the air in the flask was replaced with nitrogen.

To the mixture, 22 mg (0.10 mmol) of palladium(II) acetate was added. This mixture was stirred at 90° C. under a nitrogen stream for 7.5 hours. After that, water was added to this mixture, and an aqueous layer was subjected to extraction with ethyl acetate.

The obtained solution of the extract and an organic layer were combined, and the mixture was washed with water and saturated brine. The mixture was dried with magnesium sulfate. This mixture was separated by gravity filtration, and the filtrate was concentrated to give an oily substance. The obtained oily substance was purified by silica gel column chromatography (hexane and ethyl acetate in a ratio of 15:1). This obtained oily substance was purified by high performance liquid column chromatography (HPLC). The obtained fraction was concentrated to give a solid. The obtained oily substance was recrystallized from toluene, and a precipitated solid was collected by suction filtration. Hexane was added to the obtained solid and ultrasonic wave irradiation was performed. A solid was collected by suction filtration to give 2.2 g of a white solid, which was the object of the synthesis, in a yield of 62%.

The synthesis scheme of the above synthesis method is shown below in (B-1).

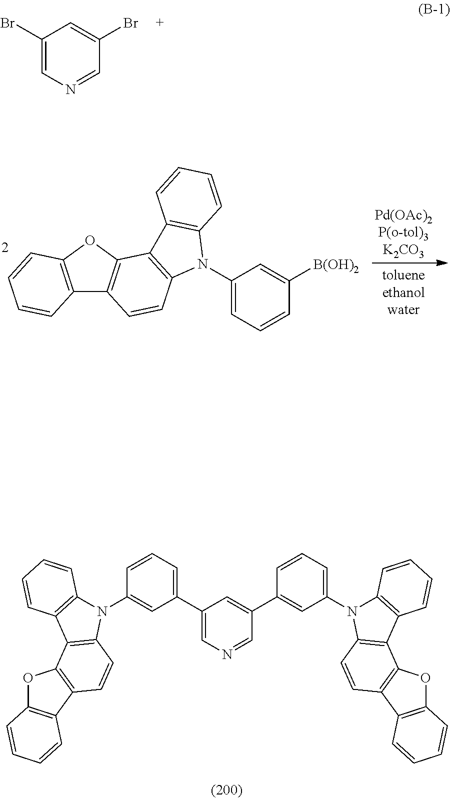

By a train sublimation method, 2.2 g of the obtained white solid was purified. In the purification by sublimation, 3,5mBFcP2Py was heated at 340° C. under the conditions where the pressure was 2.5 Pa and the argon flow rate was 5.0 mL/min. After the purification by sublimation, 1.3 g of a white powder solid of 3,5mBFcP2Py was obtained at a collection rate of 59%.

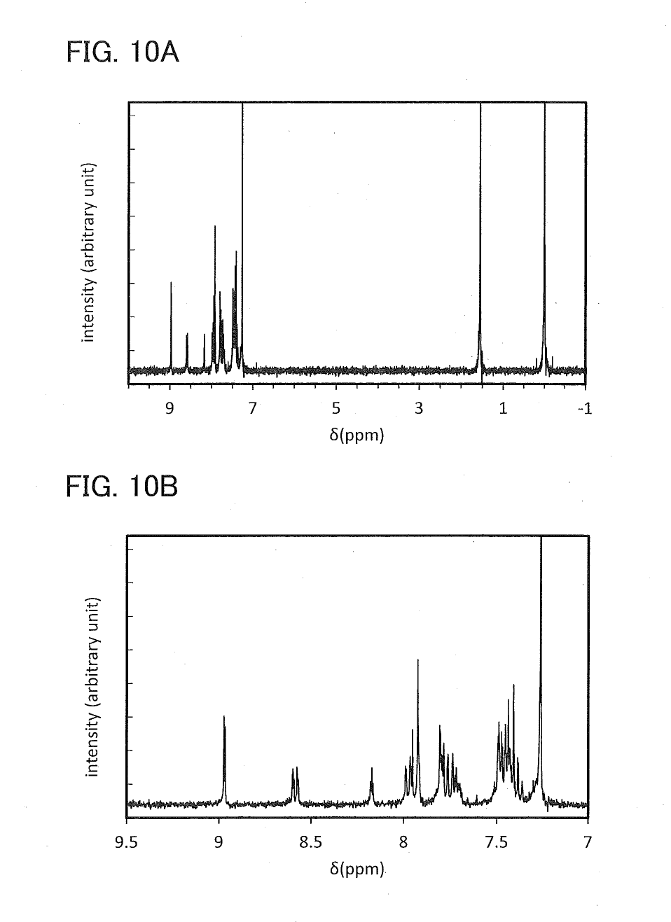

Analysis results by nuclear magnetic resonance (¹H-NM) spectroscopy of the white solid obtained by the above synthesis method are described below. FIGS. 10A and 10B are ¹H-NMR charts. FIG. 10B is a chart where the range from 7 ppm to 9.5 ppm on the horizontal axis (d) in FIG. 10A is enlarged. The results show that the organic compound of one embodiment of the present invention, 3,5mBFcP2Py (structural foimula (200)), was obtained.

¹H-NMR (CDCl₃, 300 MHz): δ (ppm)=7.36-7.51 (m, 12H), 7.68-7.81 (m, 8H), 7.92-7.99 (m, 6H), 8.17-8.18 (m, 1H), 8.59 (d, J=6.3 Hz, 2H), 8.97 (d, J=1.8 Hz, 2H).

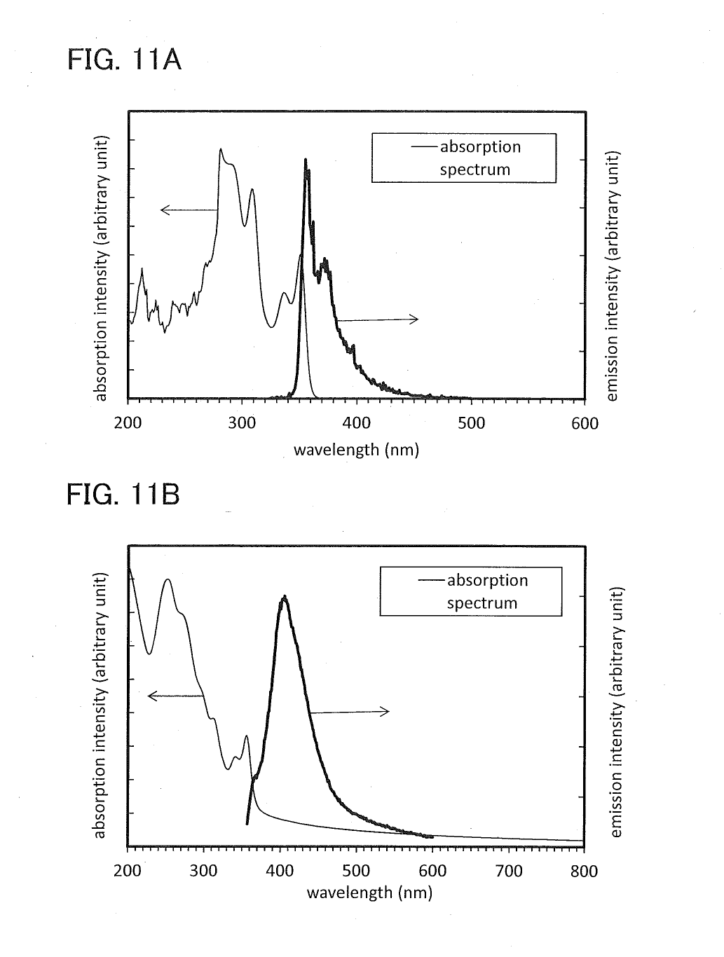

Next, ultraviolet-visible absorption spectra (hereinafter, simply referred to as “absorption spectra”) and emission spectra of 3,5mBFcP2Py in a toluene solution of 3,5mBFcP2Py and a thin film of 3,5mBFcP2Py were measured at room temperature. The spectra of the toluene solution of 3,5mBFcP2Py were measured by putting the solution in a quartz cell. The spectra of the thin film were measured with a sample prepared by evaporation of 3,5mBFcP2Py on a quartz substrate. The absorption spectra were measured with an ultraviolet-visible light spectrophotometer (V-550, manufactured by JASCO Corporation), and the emission spectra were measured with a fluorescence spectrophotometer (FS920, manufactured by Hamamatsu Photonics K.K.). FIG. 11A shows the obtained absorption and emission spectra of 3,5mBFcP2Py in the toluene solution, and FIG. 11B shows the obtained absorption and emission spectra of the thin film. The horizontal axis represents wavelength and the vertical axes represent absorption intensity and emission intensity. In each of FIGS. 11A and 11B, two solid lines are shown; the thin line represents the absorption spectrum, and the thick line represents the emission spectrum. The absorption spectrum shown in FIG. 11A was obtained by subtraction of the absorption spectra of toluene and the quartz cell from the obtained absorption spectrum. The absorption spectrum shown in FIG. 11B was obtained by subtraction of the absorption spectrum of the quartz substrate from the obtained absorption spectrum.

As shown in FIGS. 11A and 11B, 3,5mBFcP2Py, which is one embodiment of the present invention, in the toluene solution has absorption peaks at approximately 352 nm, 336 nm, 307 nm, 288 nm, and 284 nm, and emission peaks at approximately 355 nm and 374 nm. In addition, the thin film of 3,5mBFcP2Py has absorption peaks at approximately 356 nm, 341 nm, 312 nm, and 272 nm, and emission peaks at approximately 367 nm and 405 nm. Thus, it was found that absorption and emission of 3,5mBFcP2Py occur in extremely short wavelength regions.

Next, 3,5mBFcP2Py, which is one embodiment of the present invention, was analyzed by liquid chromatography mass spectrometry (LC/MS).

The analysis by LC/MS was carried out with Acquity UPLC (manufactured by Waters Corporation), and Xevo G2 Tof MS (manufactured by Waters Corporation).

In the MS, ionization was carried out by an electrospray ionization (abbreviation: ESI) method. At this time, the capillary voltage and the sample cone voltage were set to 3.0 kV and 30 V, respectively, and detection was performed in a positive mode. A component that underwent the ionization under the above-described conditions was collided with an argon gas in a collision cell to dissociate into product ions. The energy (collision energy) for the collision with argon was 70 eV. The mass range for the measurement was m/z=100 to 1200.

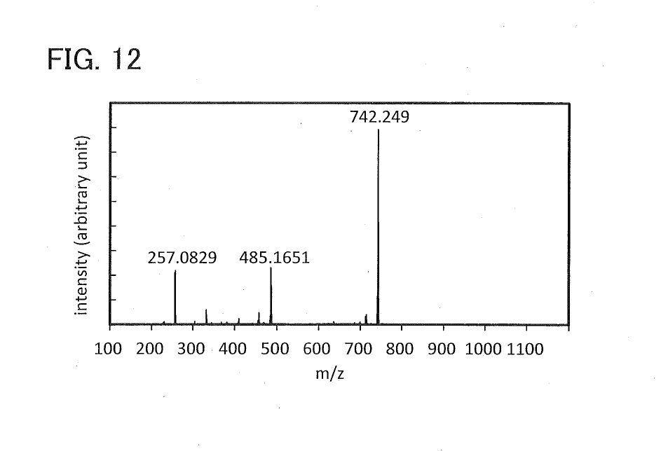

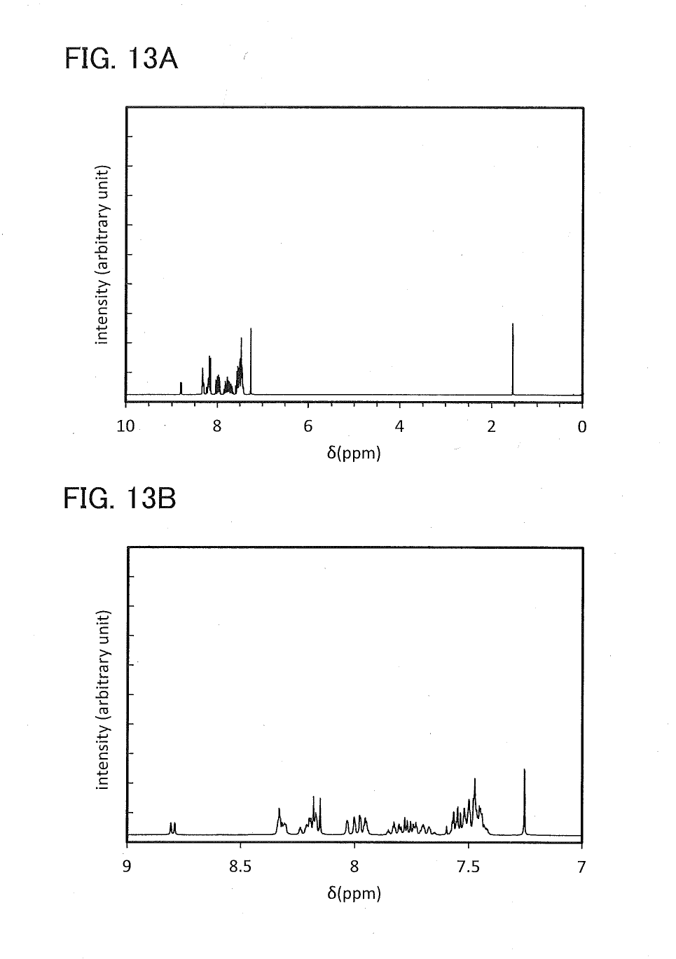

FIG. 12 shows the measurement results. The results in FIG. 12 reveal that the product ions of 3,5mBFcP2Py, which is the organic compound of one embodiment of the present invention represented by the structural formula (200), are detected mainly around m/z=742, around m/z=485, and around m/z=257. Note that the results in FIG. 12 show characteristics derived from 3,5mBFcP2Py and thus can be regarded as important data for identifying 3,5mBFcP2Py contained in a mixture.

### EXAMPLE 3

### SYNTHESIS EXAMPLE 3

In this example, a method of synthesizing an organic compound of one embodiment of the present invention, 5,5′-(2,4-pyridinediyldi-3,1-phenylene)bis-5H-benzothieno[3,2-c]carbazole (abbreviation: 2,4mBTcP2Py) (structural formula (115)), is described. Note that a structure of 2,4mBTcP2Py is shown below.

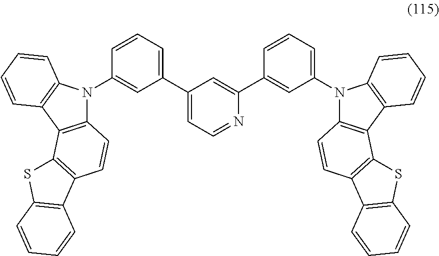 [SMILES: c1cc(-c2ccnc(-c3cccc(-n4c5ccccc5c5c6sc7ccccc7c6ccc54)c3)c2)cc(-n2c3ccccc3c3c4sc5ccccc5c4ccc32)c1]

Into a 100-mL three-neck flask were put 0.47 g (2.0 mmol) of 2,4-dibromopyridine, 2.0 g (4.2 mmol) of 2-[3-(5H-benzothieno[3,2-c]carbazol-5-yl)phenyl]-4,4,5,5-tetramethyl-1,3,2-dioxaborol ane, 61 mg (0.20 mmol) of tris(2-methylphenyl)phosphine, and 2.7 g (13 mmol) of tripotassium phosphate. The air in the flask was replaced with nitrogen.

To this mixture were added 16 mL of 1,2-dimethoxyethane (DME) and 4 mL of t-butyl alcohol, and the mixture was degassed by being stirred under reduced pressure. To the mixture, 9.0 mg (0.040 mmol) of palladium(II) acetate was added. This mixture was stirred at 90° C. under a nitrogen stream for 6 hours. Water was added to this mixture, and an aqueous layer was subjected to extraction with chloroform. The obtained solution of the extract and an organic layer were combined, and the mixture was washed with water and saturated brine. Then, the mixture was dried with magnesium sulfate. This mixture was separated by gravity filtration, and the filtrate was concentrated to give a yellow solid. This solid was purified by silica gel column chromatography (developing solvent: chloroform). The obtained fraction was concentrated to give a yellow solid. This solid was purified by high performance liquid column chromatography (HPLC) (developing solvent: chloroform). The obtained fraction was concentrated to give a white solid. This solid was recrystallized from hexane/ethyl acetate to give 0.64 g of a white solid, which was the object of the synthesis, in a yield of 40%.

The synthesis scheme of the above synthesis method is shown below in (C-1).

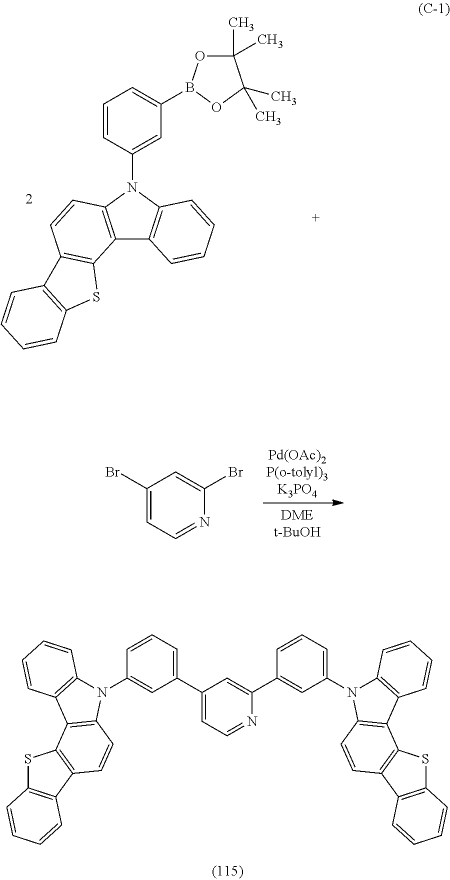

By a train sublimation method, 0.64 g of the obtained white solid was purified. In the purification by sublimation, the white solid was heated at 320° C. under the conditions where the pressure was 2.7 Pa and the argon flow rate was 5 mL/min. After the purification by sublimation, 0.56 g of a pale yellow solid was obtained at a collection rate of 88%.

Analysis results by nuclear magnetic resonance (¹H-NMR) spectroscopy of the white solid obtained by the above synthesis method are described below. FIGS. 13A and 13B are ¹H-NMR charts. FIG. 13B is a chart where the range from 7 ppm to 9 ppm on the horizontal axis (d) in FIG. 13A is enlarged. The results show that the organic compound of one embodiment of the present invention, 2,4mBTcP2Py (structural formula (115)), was obtained.

¹H-NMR (CDCl₃, 300 MHz): δ (ppm)=7.42-7.60 (m, 13H), 7.65-7.85 (in, 5H), 7.95-8.03 (m, 411), 8.15-8.24 (m, 511), 8.30-8.33 (m, 3H), 8.80 (d, J=4.8 Hz, 1H).

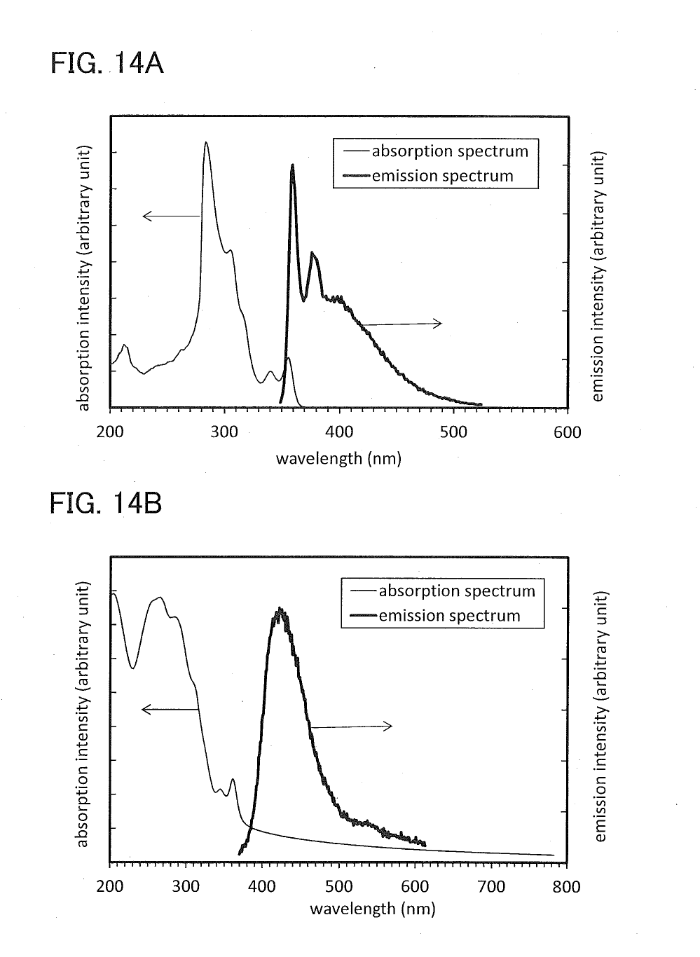

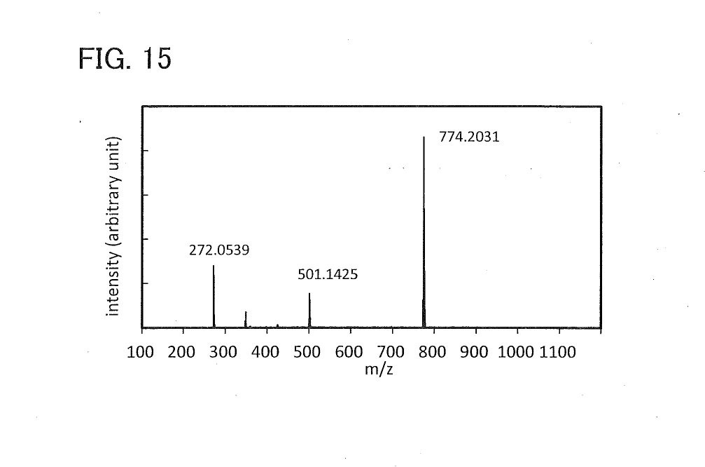

Next, ultraviolet-visible absorption spectra (hereinafter, simply referred to as “absorption spectra”) and emission spectra of 2,4mBTcP2Py in a toluene solution of 2,4mBTcP2Py and a thin film of 2,4mBTcP2Py were measured at room temperature. The spectra of the toluene solution of 2,4mBTcP2Py were measured by putting the solution in a quartz cell. The spectra of the thin film were measured with a sample prepared by evaporation of 2,4mBTcP2Py on a quartz substrate. The absorption spectra were measured with an ultraviolet-visible light spectrophotometer (V-550, manufactured by JASCO Corporation), and the emission spectra were measured with a fluorescence spectrophotometer (FS920, manufactured by Hamamatsu Photonics K.K.). FIG. 14A shows the obtained absorption and emission spectra of 2,4mBTcP2Py in the toluene solution, and FIG. 14B shows the obtained absorption and emission spectra of the thin film. The horizontal axis represents wavelength and the vertical axes represent absorption intensity and emission intensity. In each of FIGS. 14A and 14B, two solid lines are shown; the thin line represents the absorption spectrum, and the thick line represents the emission spectrum. The absorption spectrum shown in FIG. 14A was obtained by subtraction of the absorption spectra of toluene and the quartz cell from the obtained absorption spectrum. The absorption spectrum shown in FIG. 14B was obtained by subtraction of the absorption spectrum of the quartz substrate from the obtained absorption spectrum.

As shown in FIGS. 14A and 14B, 2,4mBTcP2Py, which is one embodiment of the present invention, in the toluene solution has absorption peaks at approximately 357 nm, 340 nm, 305 nm, and 283 nm, and emission peaks at approximately 359 nm and 376 nm. In addition, the thin film of 2,4mBTcP2Py has absorption peaks at approximately 361 nm, 345 nm, 312 nm, and 286 nm, and an emission peak at approximately 423 nm. Thus, it was found that absorption and emission of 2,4mBTcP2Py occur in extremely short wavelength regions.

Next, 2,4mBTcP2Py, which is one embodiment of the present invention, was analyzed by liquid chromatography mass spectrometry (LC/MS).

The analysis by LC/MS was carried out with Acquity UPLC (manufactured by Waters Corporation), and Xevo G2 Tof MS (manufactured by Waters Corporation).

In the MS, ionization was carried out by an electrospray ionization (abbreviation: ESI) method. At this time, the capillary voltage and the sample cone voltage were set to 3.0 kV and 30 V, respectively, and detection was performed in a positive mode. A component that underwent the ionization under the above-described conditions was collided with an argon gas in a collision cell to dissociate into product ions. The energy (collision energy) for the collision with argon was 70 eV. The mass range for the measurement was m/z=100 to 1200.

FIG. 15 shows the measurement results. The results in FIG. 15 reveal that the product ions of 2,4mBTcP2Py, which is the organic compound of one embodiment of the present invention represented by the structural formula (115), are detected mainly around m/z=774, around m/z=501, and around m/z=272. Note that the results in FIG. 15 show characteristics derived from 2,4mBTcP2Py and thus can be regarded as important data for identifying 2,4mBTcP2Py contained in a mixture.

### EXAMPLE 4

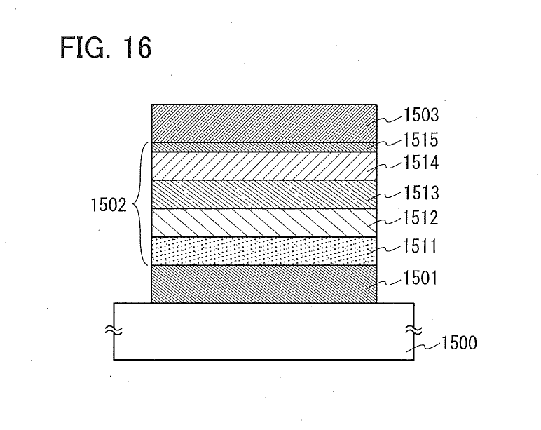

In this example, a light-emitting element 1 including the organic compound of one embodiment of the present invention is described with reference to FIG. 16. Chemical formulae of materials used in this example are shown below.

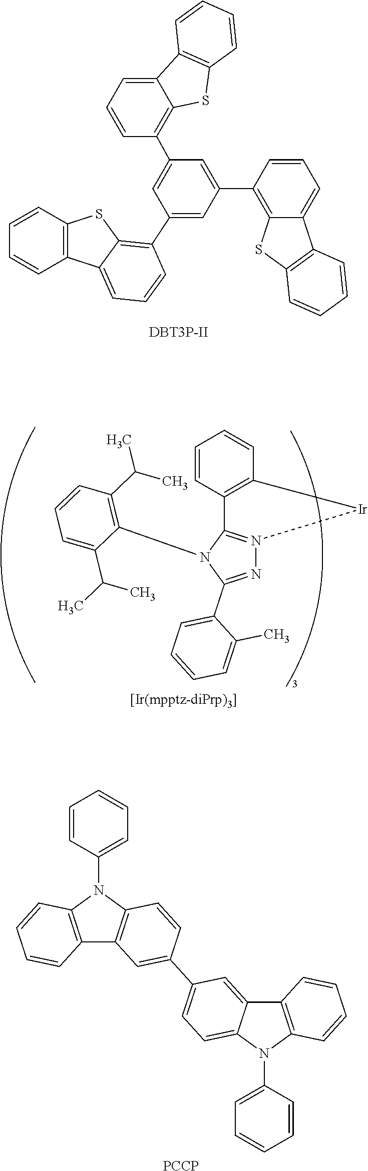

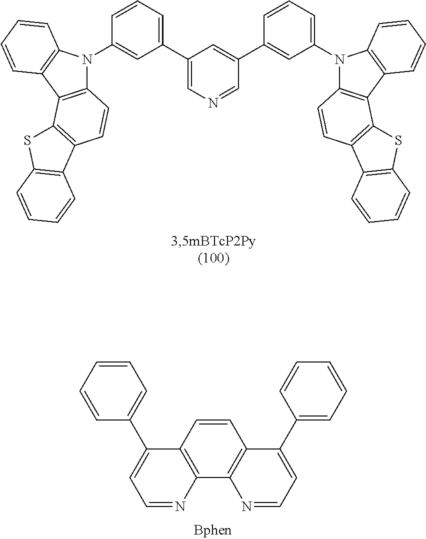 [SMILES: c1cc(-c2cncc(-c3cccc(-n4c5ccccc5c5c6sc7ccccc7c6ccc54)c3)c2)cc(-n2c3ccccc3c3c4sc5ccccc5c4ccc32)c1 ; c1ccc(-c2ccnc3c2ccc2c(-c4ccccc4)ccnc23)cc1]

### <<Fabrication of Light-Emitting Element 1>>

First, indium tin oxide containing silicon oxide (ITSO) was deposited over a glass substrate 1500 by a sputtering method, whereby a first electrode 1501 functioning as an anode was formed. Note that the thickness was set to 110 nm and the electrode area was set to 2 mm×2 mm.

Next, as pretreatment for fabricating the light-emitting element 1 over the substrate 1500, UV ozone treatment was performed for 370 seconds after washing of a surface of the substrate with water and baking that was performed at 200° C. for 1 hour.

After that, the substrate was transferred into a vacuum evaporation apparatus where the pressure had been reduced to approximately 10⁻⁴ Pa, and subjected to vacuum baking at 170° C. in a heating chamber of the vacuum evaporation apparatus for 30 minutes, and then the substrate 1500 was cooled down for approximately 30 minutes.

Next, the substrate 1500 was fixed to a holder provided in the vacuum evaporation apparatus so that a surface of the substrate over which the first electrode 1501 was formed faced downward. In this example, a case is described in which a hole-injection layer 1511, a hole-transport layer 1512, a light-emitting layer 1513, an electron-transport layer 1514, and an electron-injection layer 1515, which are included in an EL layer 1502, are sequentially formed by a vacuum evaporation method.

After reducing the pressure in the vacuum evaporation apparatus to 10⁻⁴ Pa, 1,3,5-tri(dibenzothiophen-4-yl)benzene (abbreviation: DBT3P-II) and molybdenum(VI) oxide were deposited by co-evaporation so that the mass ratio of DBT3P-II to molybdenum oxide was 4:2, whereby the hole-injection layer 1511 was formed over the first electrode 1501. The thickness was 60 nm. Note that co-evaporation is an evaporation method in which a plurality of different substances are concurrently vaporized from different evaporation sources.

Next, 9-phenyl-9H-3-(9-phenyl-9H-carbazol-3-yl)carbazole (abbreviation: PCCP) was deposited to a thickness of 20 mn by evaporation, whereby the hole-transport layer 1512 was formed.

Next, the light-emitting layer 1513 was formed over the hole-transport layer 1512.

The light-emitting layer 1513 having a stacked-layer structure was formed to a thickness of 40 nm as follows: PCCP, 5,5′-(3,5-pyridinediyldi-3,1-phenylene)bis-5H-benzothieno[3,2-c]carbazole (abbreviation: 3,5mBTcP2Py) (structural formula (100)), and tris{2-[5-(2-methylphenyl)-4-(2,6-diisopropylphenyl)-4H-1,2,4-triazol-3-yl-κN2]phenyl-κC}iridium(III) (abbreviation: [Ir(mpptz-diPrp)₃]) were deposited to a thickness of 30 nm by co-evaporation so that the mass ratio of PCCP to 3,5mBTcP2Py and [Ir(mpptz-diPrp)₃] was 1:0.3:0.06, and then 3,5mBTcP2Py and [Ir(mpptz-diPrp)₃] were deposited to a thickness of 10 nm by co-evaporation so that the mass ratio of 3,5mBTcP2Py to[Ir(mpptz-diPrp)₃] was 1:0.06.

Next, the electron-transport layer 1514 was formed over the light-emitting layer 1513.

First, 3,5mBTcP2Py was deposited to a thickness of 10 nm by evaporation and then bathophenanthroline (abbreviation: Bphen) was deposited to a thickness of 20 nm by evaporation, whereby the electron-transport layer 1514 was formed.

Next, lithium fluoride was deposited to a thickness of 1 mn over the electron-transport layer 1514 by evaporation, whereby the electron-injection layer 1515 was formed.

Finally, aluminum was deposited to a thickness of 200 nm over the electron-injection layer 1515, whereby a second electrode 1503 functioning as a cathode was formed. Through the above-described steps, the light-emitting element 1 was fabricated. Note that in all the above evaporation steps, evaporation was performed by a resistance-heating method.

Table 1 shows the element structure of the light-emitting element 1 fabricated as described above.

TABLE 1

| | Light-emitting element 1 |
|---|---|
| First electrode | ITSO (110 nm) |
| Hole-injection layer | DBT3P-II:MoOx (4:2, 60 nm) |
| Hole-transport layer | PCCP (20 nm) |
| Light-emitting layer | PCCP:3,5mBTcP2Py:[Ir(mpptz-diPrp)₃] |
| | (1:0.3:0.06, 30 nm) |
| | 3,5mBTcP2Py:[Ir(mpptz-diPrp)₃] |
| | (1:0.06, 10 nm) |
| Electron-transport layer | 3,5mBTcP2Py (10 nm) |
| | Bphen (20 nm) |
| Electron-injection layer | LiF (1 nm) |
| Second electrode | Al (200 nm) |

The fabricated light-emitting element 1 was sealed in a glove box containing a nitrogen atmosphere so as not to be exposed to the air (specifically, a sealant was applied to surround the element, and at the time of sealing, first, UV treatment was performed and then heat treatment was perfoimed at 80° C. for 1 hour).

### <<Operation Characteristics of Light-Emitting Element 1>>

Operation characteristics of the fabricated light-emitting element 1 were measured. Note that the measurements were carried out at room temperature (in an atmosphere kept at 25° C.).

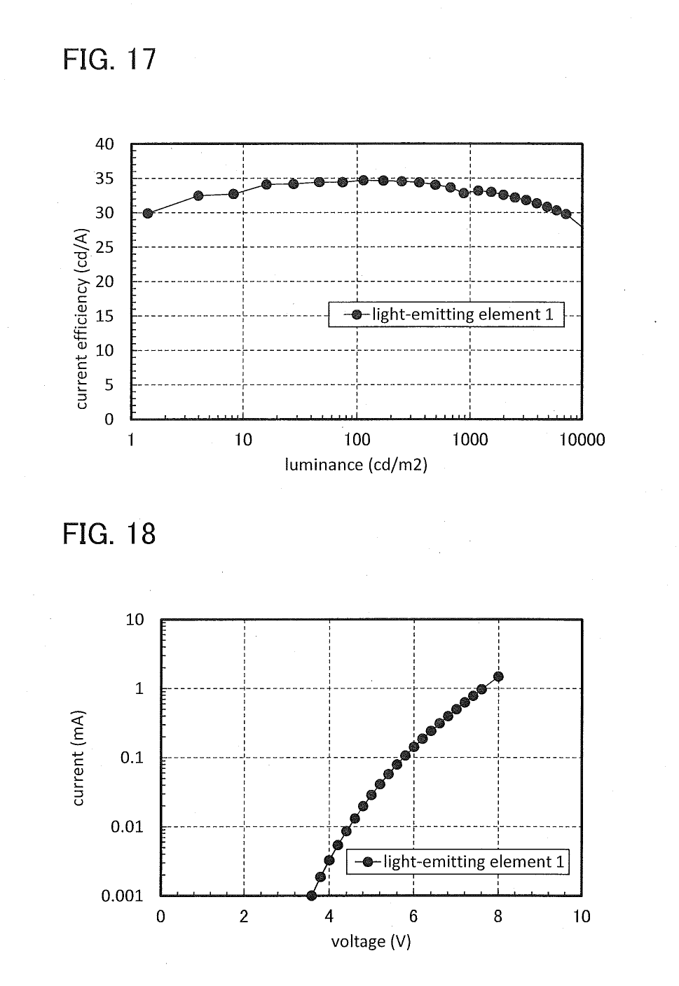

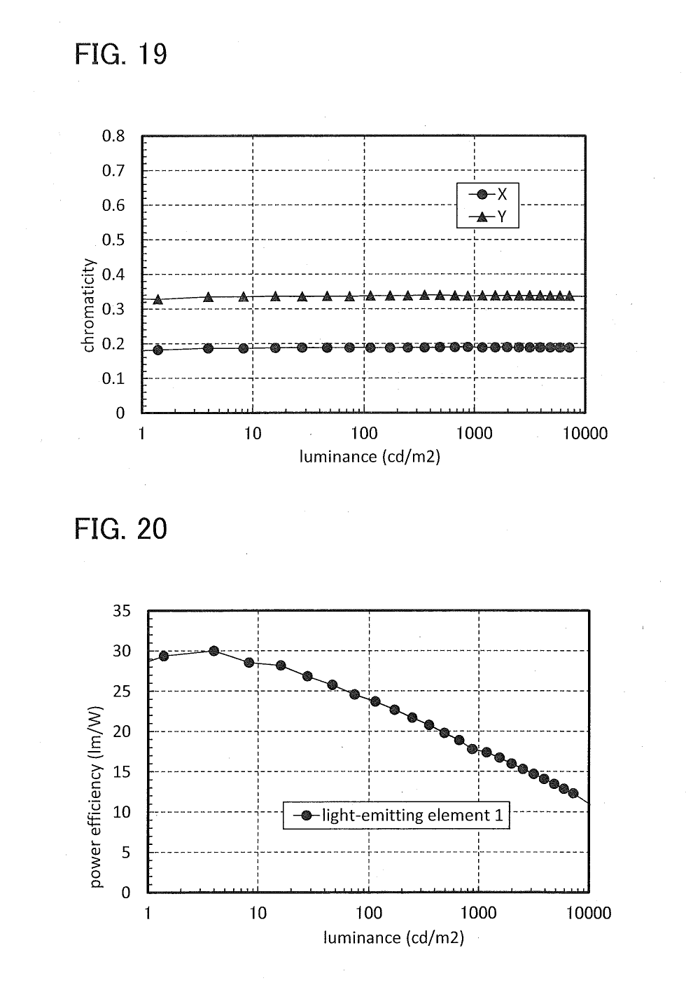

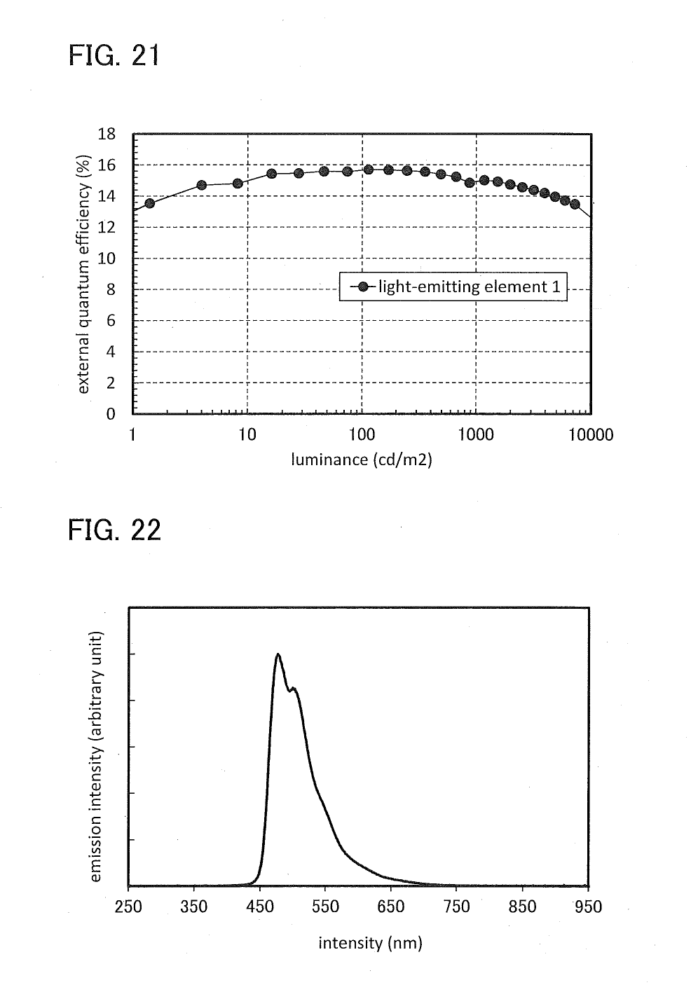

FIG. 17 shows luminance-current efficiency characteristics of the light-emitting element 1. FIG. 18 shows voltage-current characteristics of the light-emitting element 1. FIG. 19 shows luminance-chromaticity characteristics of the light-emitting element 1. FIG. 20 shows luminance-power efficiency characteristics of the light-emitting element 1. FIG. 21 shows luminance-external quantum efficiency characteristics of the light-emitting element 1.

These results reveal that the light-emitting element 1 of one embodiment of the present invention has high efficiency. Table 2 shows initial values of main characteristics of the light-emitting element 1 at a luminance of approximately 1000 cd/m². Note that blue light emission originating from [Ir(mpptz-diPrp)₃], which was used as a guest material of the light-emitting layer, was obtained from the light-emitting element 1.

TABLE 2

| | Light-emitting element 1 |
|---|---|
| Voltage (V) | 5.6 |
| Current (mA) | 0.079 |
| Current density (mA/cm²) | 2.0 |
| Chromaticity (x, y) | (0.19, 0.34) |
| Luminance (cd/m²) | 670 |
| Current efficiency (cd/A) | 34 |
| Power efficiency (lm/W) | 19 |
| External quantum efficiency (%) | 15 |

FIG. 22 shows an emission spectrum when a current at a current density of 25 mA/cm² was supplied to the light-emitting element 1. As shown in FIG. 22, the emission spectrum of the light-emitting element 1 has a peak at approximately 476 nm, indicating that the peak is derived from emission from [Ir(mpptz-diPrp)₃], which was used as the guest material.

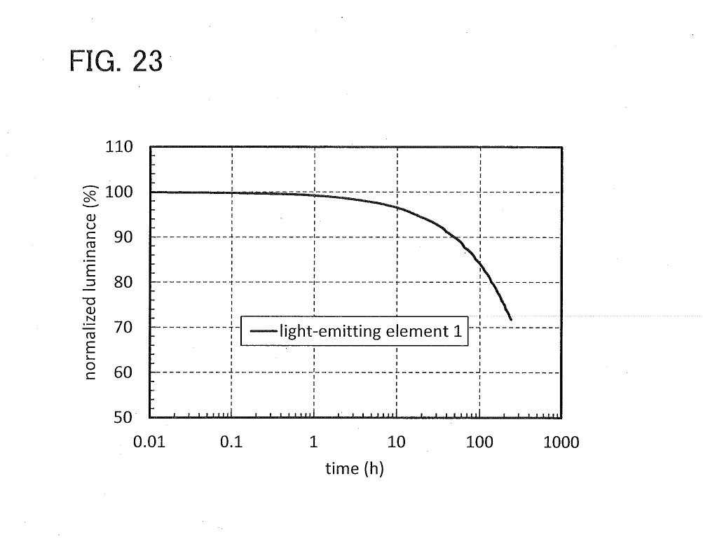

Next, the light-emitting element 1 was subjected to a reliability test. Results of the reliability test are shown in FIG. 23. In FIG. 23, the vertical axis represents normialized luminance (%) with an initial luminance of 100% and the horizontal axis represents driving time (h) of the element. Note that in the reliability test, the light-emitting element 1 was driven under the conditions where the initial luminance was set to 1000 cd/m² and the current density was constant. As a result, the light-emitting element 1 kept about 84% of the initial luminance after 100 hours elapsed.

As revealed from the results, the light-emitting element 1, in which the organic compound (3,5mBTcP2Py) of one embodiment of the present invention is used in the light-emitting layer, is highly reliable. Furthermore, the use of the organic compound of one embodiment of the present invention in a light-emitting element can reduce the drive voltage and reduce the power consumption accordingly.

### EXAMPLE 5

In this example, as in Example 4, a light-emitting element 2 including the organic compound of one embodiment of the present invention is described with reference to FIG. 16. Chemical formulae of materials used in this example are shown below.

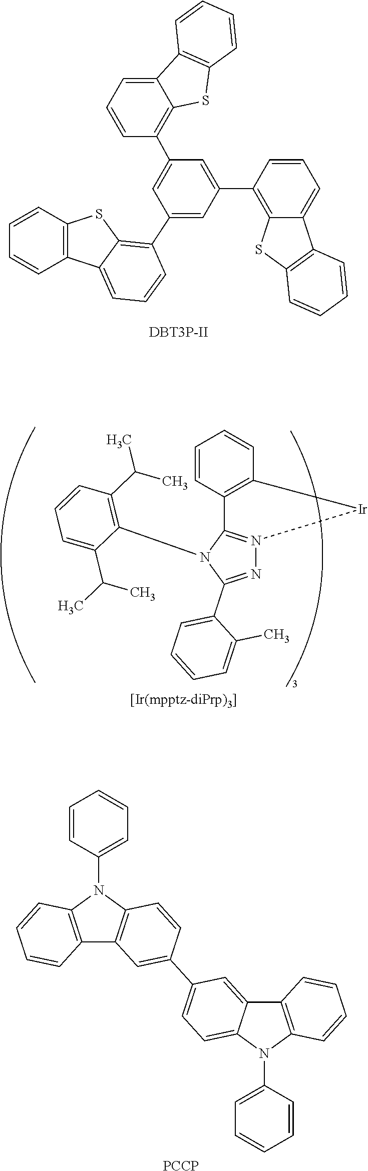

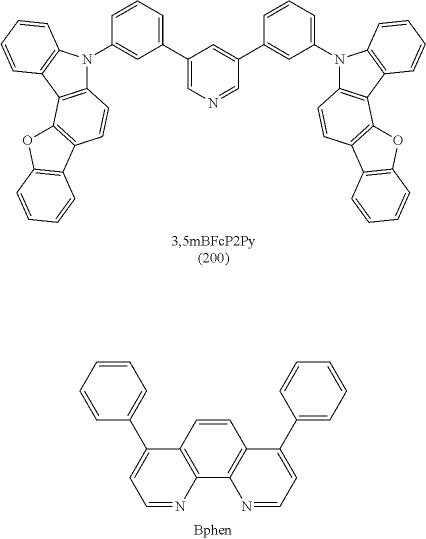 [SMILES: c1cc(-c2cncc(-c3cccc(-n4c5ccccc5c5c6oc7ccccc7c6ccc54)c3)c2)cc(-n2c3ccccc3c3c4oc5ccccc5c4ccc32)c1 ; c1ccc(-c2ccnc3c2ccc2c(-c4ccccc4)ccnc23)cc1]

### <<Fabrication of Light-Emitting Element 2>>

First, indium tin oxide containing silicon oxide (ITSO) was deposited over a glass substrate 1500 by a sputtering method, whereby a first electrode 1501 functioning as an anode was formed. Note that the thickness was set to 110 nm and the electrode area was set to 2 mm×2 mm.

Next, as pretreatment for fabricating the light-emitting element 2 over the substrate 1500, UV ozone treatment was performed for 370 seconds after washing of a surface of the substrate with water and baking that was performed at 200° C. for 1 hour.

After that, the substrate was transferred into a vacuum evaporation apparatus where the pressure had been reduced to approximately 10⁻⁴ Pa, and subjected to vacuum baking at 170° C. in a heating chamber of the vacuum evaporation apparatus for 30 minutes, and then the substrate 1500 was cooled down for approximately 30 minutes.

Next, the substrate 1500 was fixed to a holder provided in the vacuum evaporation apparatus so that a surface of the substrate over which the first electrode 1501 was formed faced downward. In this example, a case is described in which a hole-injection layer 1511, a hole-transport layer 1512, a light-emitting layer 1513, an electron-transport layer 1514, and an electron-injection layer 1515, which are included in an EL layer 1502, are sequentially formed by a vacuum evaporation method.

After reducing the pressure in the vacuum evaporation apparatus to 10⁻⁴ Pa, 1,3,5-tri(dibenzothiophen-4-yl)benzene (abbreviation: DBT3P-II) and molybdenum(VI) oxide were deposited by co-evaporation so that the mass ratio of DBT3P-II to molybdenum oxide was 4:2, whereby the hole-injection layer 1511 was formed over the first electrode 1501. The thickness was 60 mu. Note that co-evaporation is an evaporation method in which a plurality of different substances are concurrently vaporized from different evaporation sources.

Next, 9-phenyl-9H-3-(9-phenyl-9H-carbazol-3-yl)carbazole (abbreviation: PCCP) was deposited to a thickness of 20 nm by evaporation, whereby the hole-transport layer 1512 was formed.

Next, the light-emitting layer 1513 was formed over the hole-transport layer 1512.

The light-emitting layer 1513 having a stacked-layer structure was formed to a thickness of 40 nm as follows: PCCP, 5,5′-(3 ,5-pyridinediyldi-3,1-phenylene)bis-5H-benzofuro[3,2-c]carbazole (abbreviation: 3,5mBFcP2Py) (structural formula (200)), and tris{2-[5-(2-methylphenyl)-4-(2,6-diisopropylphenyl)-4H-1,2,4-triazol-3-yl-κN2]phenyl-κC}iridium(III) (abbreviation: [Ir(mpptz-diPrp)₃]) were deposited to a thickness of 30 mn by co-evaporation so that the mass ratio of PCCP to 3,5mBFcP2Py and [Ir(mpptz-diPrp)₃] was 1:0.3:0.06, and then 3,5mBFcP2Py and [Ir(mpptz-diPrp)₃] were deposited to a thickness of 10 nm by co-evaporation so that the mass ratio of 3,5mBFcP2Py to[Ir(mpptz-diPrp)₃] was 1:0.06.

Next, the electron-transport layer 1514 was formed over the light-emitting layer 1513.

First, 3,5mBFcP2Py was deposited to a thickness of 10 mn by evaporation and then bathophenanthroline (abbreviation: Bphen) was deposited to a thickness of 20 nm by evaporation, whereby the electron-transport layer 1514 was formed.

Next, lithium fluoride was deposited to a thickness of 1 nm over the electron-transport layer 1514 by evaporation, whereby the electron-injection layer 1515 was formed.

Finally, aluminum was deposited to a thickness of 200 nm over the electron-injection layer 1515, whereby a second electrode 1503 functioning as a cathode was formed. Through the above-described steps, the light-emitting element 2 was fabricated. Note that in all the above evaporation steps, evaporation was performed by a resistance-heating method.

Table 3 shows the element structure of the light-emitting element 2 fabricated as described above.

TABLE 3

| | Light-emitting element 2 |
|---|---|
| First electrode | ITSO (110 nm) |
| Hole-injection layer | DBT3P-II:MoOx (4:2, 60 nm) |
| Hole-transport layer | PCCP (20 nm) |
| Light-emitting layer | PCCP:3,5mBFcP2Py:[Ir(mpptz-diPrp)₃] |
| | (1:0.3:0.06, 30 nm) |
| | 3,5mBFcP2Py:[Ir(mpptz-diPrp)₃] |
| | (1:0.06, 10 nm) |
| Electron-transport layer | 3,5mBFcP2Py (10 nm) |
| | Bphen (20 nm) |
| Electron-injection layer | LiF (1 nm) |
| Second electrode | Al (200 nm) |

The fabricated light-emitting element 2 was sealed in a glove box containing a nitrogen atmosphere so as not to be exposed to the air (specifically, a sealant was applied to surround the element, and at the time of sealing, first, UV treatment was performed and then heat treatment was performed at 80° C. for 1 hour).

### <<Operation Characteristics of Light-Emitting Element 2>>

Operation characteristics of the fabricated light-emitting element 2 were measured. Note that the measurements were carried out at room temperature (in an atmosphere kept at 25° C.).

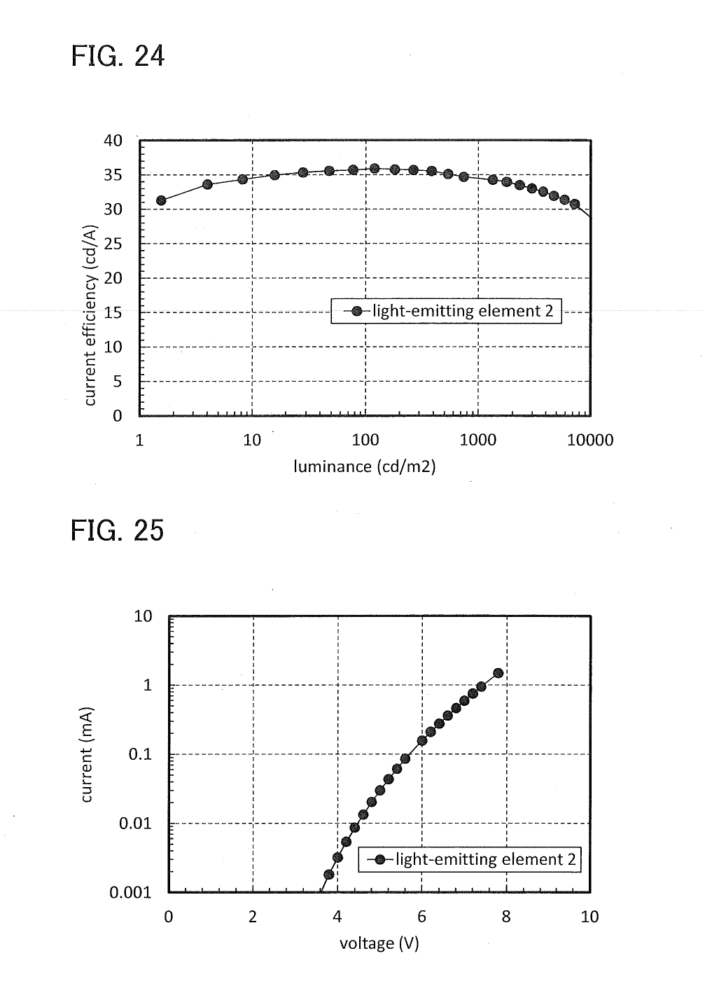

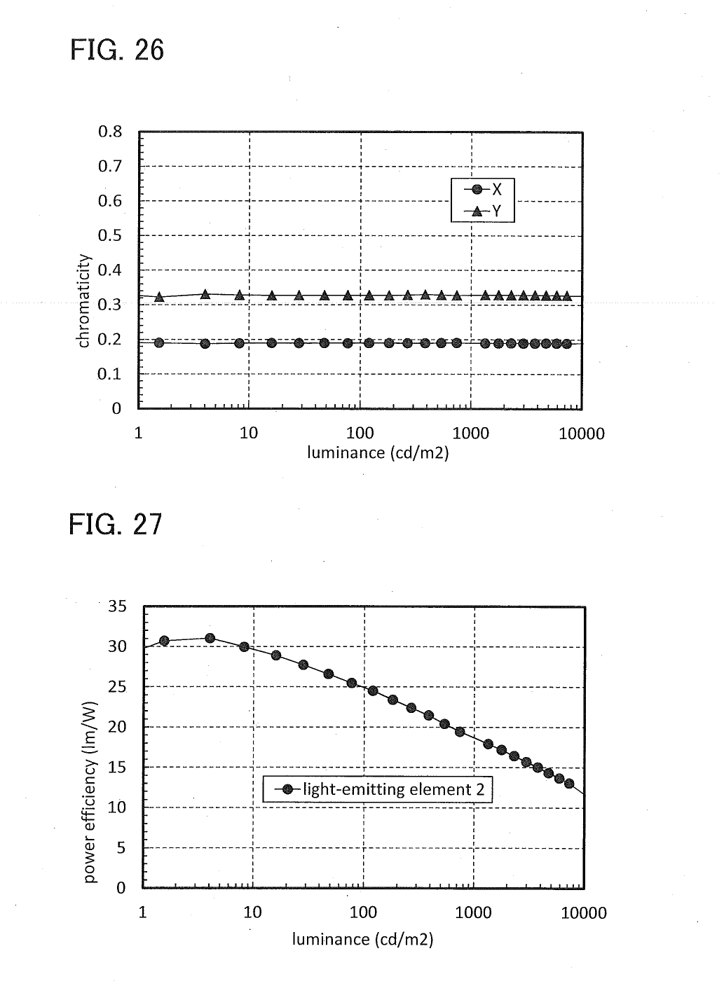

FIG. 24 shows luminance-current efficiency characteristics of the light-emitting element 2. FIG. 25 shows voltage-current characteristics of the light-emitting element 2. FIG. 26 shows luminance-chromaticity characteristics of the light-emitting element 2. FIG. 27 shows luminance-power efficiency characteristics of the light-emitting element 2. FIG. 28 shows luminance-external quantum efficiency characteristics of the light-emitting element 2.

These results demonstrate the high efficiency of the light-emitting element 2 of one embodiment of the present invention. Table 4 shows initial values of main characteristics of the light-emitting element 2 at a luminance of approximately 1000 cd/m². Note that blue light emission originating from [Ir(mpptz-diPrp)₃], which was used as a guest material of the light-emitting layer, was obtained from the light-emitting element 2.

TABLE 4

| | Light-emitting element 2 |
|---|---|
| Voltage (V) | 5.6 |
| Current (mA) | 0.086 |
| Current density (mA/cm²) | 2.1 |
| Chromaticity (x, y) | (0.19, 0.33) |
| Luminance (cd/m²) | 740 |
| Current efficiency (cd/A) | 35 |
| Power efficiency (lm/W) | 19 |
| External quantum efficiency (%) | 16 |

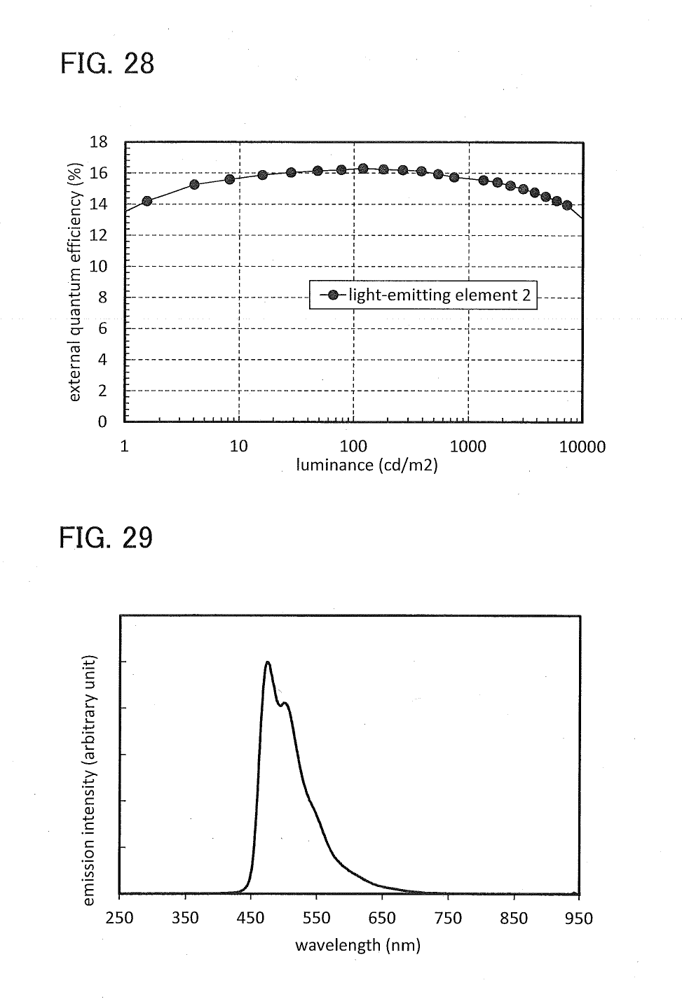

FIG. 29 shows an emission spectrum when a current at a current density of 25 mA/cm² was supplied to the light-emitting element 2. As shown in FIG. 29, the emission spectrum of the light-emitting element 2 has a peak at approximately 474 nm, indicating that the peak is derived from emission from [Ir(mpptz-diPrp)₃], which was used as the guest material.

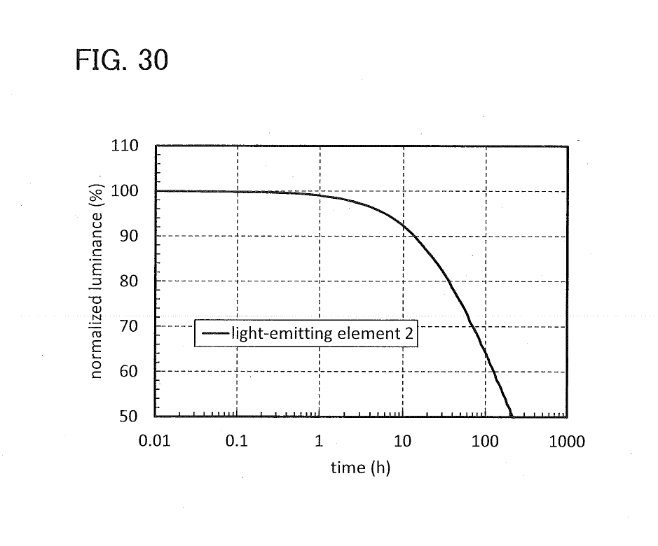

Next, the light-emitting element 2 was subjected to a reliability test. Results of the reliability test are shown in FIG. 30. In FIG. 30, the vertical axis represents normalized luminance (%) with an initial luminance of 100% and the horizontal axis represents driving time (h) of the element. Note that in the reliability test, the light-emitting element 2 was driven under the conditions where the initial luminance was set to 1000 cd/m² and the current density was constant. As a result, the light-emitting element 2 kept about 64% of the initial luminance after 100 hours elapsed.

As revealed from the results, the light-emitting element 2, in which the organic compound (3,5mBFcP2Py) of one embodiment of the present invention is used in the light-emitting layer, is highly reliable. Furthermore, the use of the organic compound of one embodiment of the present invention in the light-emitting element can reduce the drive voltage and reduce the power consumption accordingly.

### EXAMPLE 6

### SYNTHESIS EXAMPLE 4

In this example, a method of synthesizing an organic compound of one embodiment of the present invention, 5,5′-(4,6-pyrimidinediyldi-3,1-phenylene)bis-5H-benzothieno[3,2-c]carbazole (abbreviation: 4,6mBTcP2Pm) (structural formula (112)), is described. Note that a structure of 4,6mBTcP2Pm is shown below.

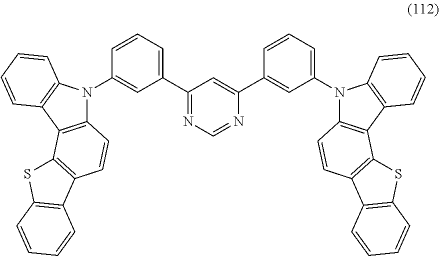 [SMILES: c1cc(-c2cc(-c3cccc(-n4c5ccccc5c5c6sc7ccccc7c6ccc54)c3)ncn2)cc(-n2c3ccccc3c3c4sc5ccccc5c4ccc32)c1]

Into a 100-mL three-neck flask were put 0.75 g (3.2 mmol) of 4,6-dibromopyridine, 3.3 g (6.9 mmol) of 2-[3-(5H-benzothieno[3,2-c]carbazol-5-yl)phenyl]-4,4,5,5-tetramethyl-1,3,2-dioxaborol ane, and 96 mg (0.32 mmol) of tris(2-methylphenyl)phosphine. The air in the flask was replaced with nitrogen. To this mixture were added 7 mL of a 2M aqueous solution of potassium carbonate, 16 mL of toluene, and 5 mL of ethanol. The mixture was degassed by being stirred under reduced pressure. To this mixture, 14 mg (0.062 mmol) of palladium(II) acetate was added. The mixture was stirred at 90° C. under a nitrogen stream for 16 hours.

After that, a precipitated solid was collected by suction filtration. Chloroform was added to this solid, and irradiation with ultrasonic waves was performed; then, a solid was collected by suction filtration. Toluene was added to this solid, and irradiation with ultrasonic waves was performed. A solid was collected by suction filtration to give 1.9 g of a brown solid, which was the object of the synthesis, in a yield of 79%.

The synthesis scheme of the above synthesis method is shown below in (D-1).

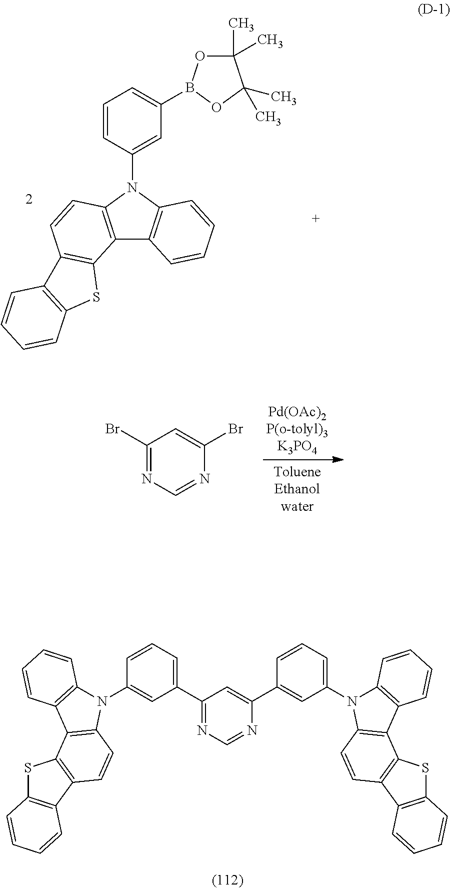

By a train sublimation method, 1.9 g of the obtained brown solid was purified. In the purification by sublimation, the brown solid was heated at 360° C. under the conditions where the pressure was 3.0 Pa and the argon flow rate was 5.0 mL/min. After the purification by sublimation, 0.76 g of a yellow solid was obtained at a collection rate of 40%.

By a train sublimation method, 0.76 g of the obtained yellow solid was again purified. In the purification by sublimation, the yellow solid was heated at 360° C. under the conditions where the pressure was 3.0 Pa and the argon flow rate was 5.0 mL/min. After the purification by sublimation, 0.58 g of a yellow solid was obtained at a collection rate of 90%.

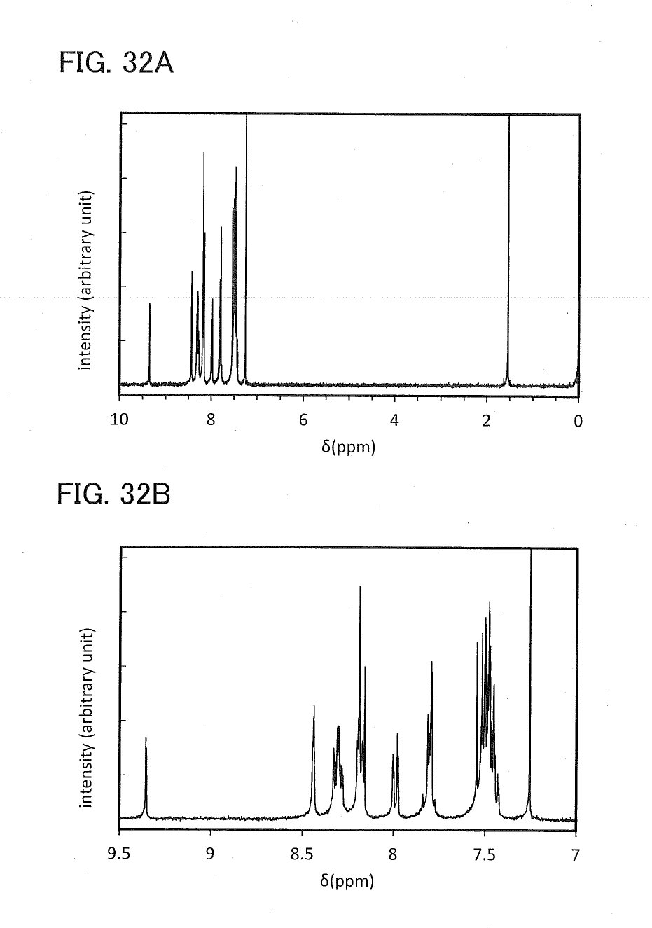

Analysis results by nuclear magnetic resonance (¹H-NMR) spectroscopy of the obtained yellow solid are described below. FIGS. 32A and 32B are ¹H-NMR charts. The results show that the organic compound of one embodiment of the present invention, 4,6mBTcP2Pm (structural formula (112)), was obtained.

¹H-NMR (CDCl₃, 300 MHz): δ (ppm)=7.43-7.55 (m, 12H), 7.77-7.84 (m, 411), 7.99 (d, J=8.7 Hz, 2H), 8.16-8.20 (m, 5H), 8.28-8.33 (m, 4H), 8.44 (s, 2H), 9.36 (d, J=0.9 Hz, 1H).

This application is based on Japanese Patent Application serial no. 2014-181112 filed with the Japan Patent Office on Sep. 5, 2014, the entire contents of which are hereby incorporated by reference.

What is claimed is:

1. An organic compound comprising:
a π-electron deficient divalent heteroaromatic group; and
a plurality of sets each comprising:
an arylene group; and
a heterocyclic skeleton that is connected to the heteroaromatic group via the arylene group and comprises any of a benzothienocarbazolyl group, a benzofurocarbazolyl group, and an indenocarbazolyl group.

2. An organic compound represented by a general formula (G1), 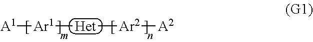
wherein, in the general foirmula (G1):
Het represents a substituted or unsubstituted π-electron deficient divalent heteroaromatic group having 4 to 36 carbon atoms;
Ar¹ and Ar² each independently represent a substituted or unsubstituted arylene group having 6 to 25 carbon atoms;
m and n each independently represent a natural number greater than or equal to 1 and less than or equal to 5; and
A¹ and A² each independently represent any of a substituted or unsubstituted benzothienocarbazolyl group, a substituted or unsubstituted benzofurocarbazolyl group, and a substituted or unsubstituted indenocarbazolyl group.

3. The organic compound according to claim 2,
wherein Het represents a substituted or unsubstituted π-electron deficient divalent heteroarornatic group having 4 to 18 carbon atoms.

4. The organic compound according to claim 2,
wherein Het represents a substituted or unsubstituted π-electron deficient divalent monocyclic nitrogen-containing heteroaromatic group.

5. The organic compound according to claim 2,
wherein Het represents any of substituted or unsubstituted divalent pyridine, substituted or unsubstituted divalent pyrimidine, substituted or unsubstituted divalent pyrazine, substituted or unsubstituted divalent triazine, substituted or unsubstituted divalent bipyridine, substituted or unsubstituted divalent quinoxaline, and substituted or unsubstituted divalent dibenzoquinoxaline.

6. The organic compound according to claim 2,
wherein the organic compound represented by the general formula (G1) has a structure represented by any one of the following structural formulae (100), (112), (115), and (200),

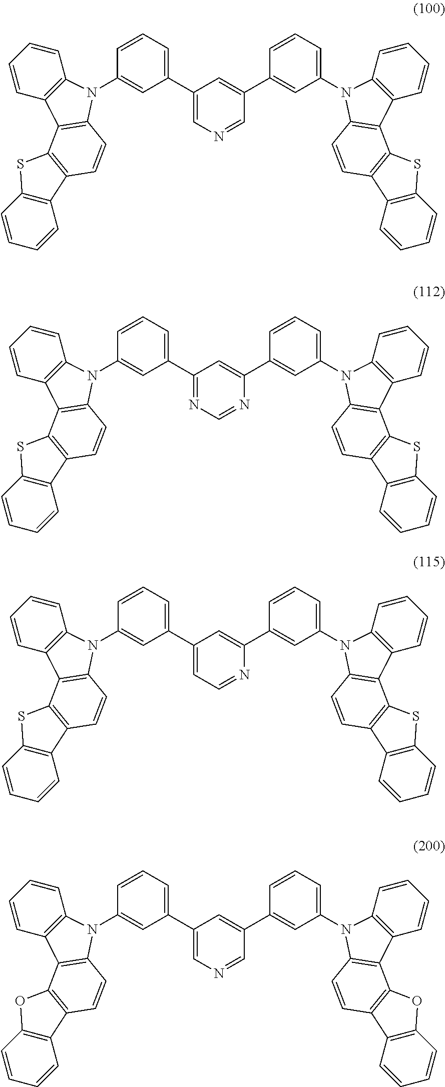

7. A light-emitting element comprising the organic compound according to claim 2.

8. A light-emitting device comprising:
the light-emitting element according to claim 7; and
a transistor or a substrate.

9. An electronic device comprising:
the light-emitting device according to claim 8; and
a microphone, a camera, a button for operation, an external connection portion, or a speaker.

10. A lighting device comprising:
the light-emitting device according to claim 8; and
a housing, a cover, or a support.

11. An organic compound represented by a general formula (G2), 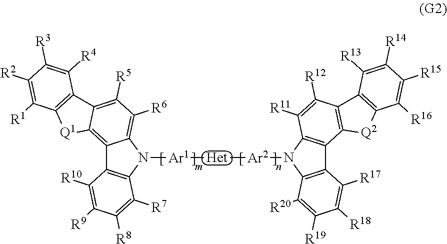
wherein, in the general formula (G2):
Het represents any of substituted or unsubstituted divalent pyridine, substituted or unsubstituted divalent pyrimidine, substituted or unsubstituted divalent pyrazine, substituted or unsubstituted divalent triazine, substituted or unsubstituted divalent bipyridine, substituted or unsubstituted divalent quinoxaline, and substituted or unsubstituted divalent dibenzoquinoxaline;
Ar¹ and Ar² each independently represent a substituted or unsubstituted arylene group having 6 to 25 carbon atoms;
m and n each independently represent a natural number greater than or equal to 1 and less than or equal to 5;
Q¹ and Q² each independently represent any of a sulfur atom, an oxygen atom, and a substituted or unsubstituted carbon atom; and
R¹ to R²⁰ each independently represent any of hydrogen, an alkyl group having 1 to 6 carbon atoms, and an aryl group having 6 to 13 carbon atoms.

12. A light-emitting element comprising the organic compound according to claim 11.

13. A light-emitting device comprising:
the light-emitting element according to claim 12; and
a transistor or a substrate.

14. An organic compound represented by a general formula (G3), 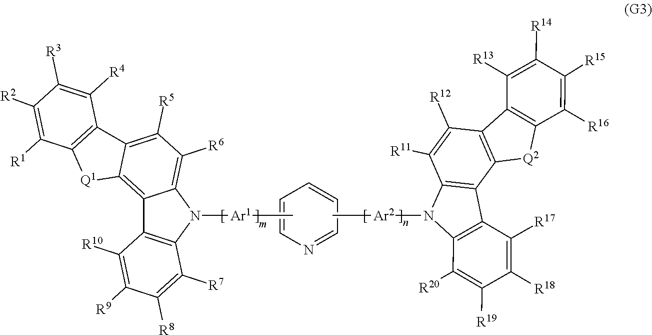
wherein, in the general formula (G3):
Ar¹ and Ar² each independently represent a substituted or unsubstituted arylene group having 6 to 25 carbon atoms;
m and n each independently represent a natural number greater than or equal to 1 and less than or equal to 5;
Q¹ and Q² each independently represent any, of a sulfur atom, an oxygen atom, and a substituted or unsubstituted carbon atom; and
R¹ to R²⁰ each independently represent any of hydrogen, an alkyl group having 1 to 6 carbon atoms, and an aryl group having 6 to 13 carbon atoms.

15. A light-emitting element comprising the organic compound according to claim 14.

16. A light-emitting device comprising:
the light-emitting element according to claim 15; and
a transistor or a substrate.

17. An organic compound represented by a general formula (G4), 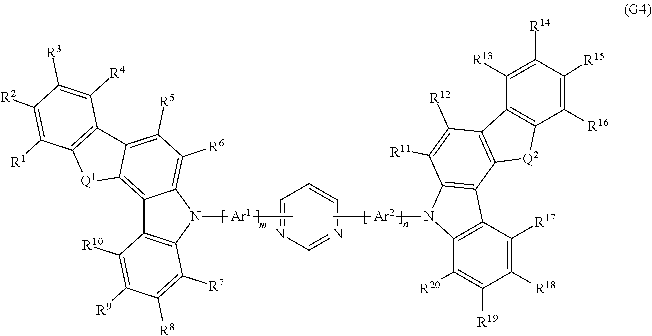
wherein, in the general formula (G4):
Ar¹ and Ar² each independently represent a substituted or unsubstituted arylene group having 6 to 25 carbon atoms;
m and n each independently represent a natural number greater than or equal to 1 and less than or equal to 5;
Q¹ and Q² each independently represent any of a sulfur atom, an oxygen atom, and a substituted or unsubstituted carbon atom; and
R¹ to R²⁰ each independently represent any of hydrogen, an alkyl group having 1 to 6 carbon atoms, and an aryl group having 6 to 13 carbon atoms.

18. A light-emitting element comprising the organic compound according to claim 17.

19. A light-emitting device comprising:
the light-emitting element according to claim 18; and
a transistor or a substrate.
